(19) United States
(12) Patent Application Publication
(10) Pub. No.: US 2015/0260723 A1
(43) Pub. Date: Sep. 17, 2015

(54) COMPANION DIAGNOSTICS FOR TEC FAMILY KINASE INHIBITOR THERAPY

(71) Applicant: PHARMACYCLICS, INC., Sunnyvale, CA (US)

(72) Inventors: Betty Y. Chang, Cupertino, CA (US); Stella Chang, San Jose, CA (US)

(21) Appl. No.: 14/434,015

(22) PCT Filed: Oct. 11, 2013
(86) PCT No.: PCT/US13/64688

§ 371 Date: Apr. 7, 2015

Related U.S. Application Data
(60) Provisional application No. 61/712,675, filed on Oct. 11, 2012.

(51) Int. Cl. G01N 33/574; G01N 33/58; C07D 519/00

(52) U.S. Cl. CPC G01N 33/57496; C07D 519/00; G01N 33/581; G01N 2333/912

(57) ABSTRACT

The invention provides methods, assays and systems for determining the efficacy of a TEC family kinase inhibitor on a target kinase. The methods, assays and systems relate to determining the occupancy of a target kinase by a TEC family kinase inhibitor (e.g., BTK inhibitors). Such quantitative measurements are used to inform therapeutic treatment and the over-all health care management of a subject. For example, diagnostic kits for diagnosing, prognosing, and monitoring a disease or indication benefiting from treatment with a TEC family kinase inhibitor are provided. In another example, diagnostic kits for identifying responders to TEC family kinase inhibitor therapy, determining therapeutic regimens, and detecting resistance to TEC family kinase inhibitor also are provided.

### RELATED APPLICATION

The present application claims the benefit of priority from U.S. Provisional Patent Application No. 61/712,675, filed Oct. 11, 2012, which is herein incorporated by reference in its entirety.

### FIELD OF THE INVENTION

Described herein are companion diagnostic methods and kits for use in combination with a therapy comprising administration of a TEC family kinase inhibitor.

### BACKGROUND OF THE INVENTION

The TEC kinase family is a subfamily of non-receptor protein-tyrosine kinases (PTKs). The TEC kinase family is composed of five members, TEC, BTK (Bruton's Tyrosine Kinase), ITK (interleukin-2-inducible T-cell kinase)/EMT/TSK, BMX and TXK/RLK. A characteristic feature of this family is the presence of a pleckstrin homology (PH) domain, which is known to bind phosphoinositides. The TEC family kinases participate in phosphotyrosine-mediated and phospholipid-mediated signaling systems. Many TEC family proteins are abundantly expressed in hematopoietic tissues, and play important roles in the growth and differentiation processes of blood cells. Mutations in the BTK gene cause X chromosome-linked agammaglobulinemia (XLA) in humans and X chromosome-linked immunodeficiency (Xid) in mice, indicating that BTK activity is indispensable for B-cell ontogeny. ITK is functionally important for the development and effector function of Th2 and Th17 cells. In addition, TEC family kinases have been shown to be involved in the intracellular signaling mechanisms of cytokine receptors, lymphocyte surface antigens, heterotrimeric G-protein-coupled receptors and integrin molecules Inhibitors of TEC kinases have been developed for the treatment of a variety of diseases associated with activation of TEC family kinases, including cancer, autoimmune disorders, and inflammatory diseases.

### SUMMARY OF THE INVENTION

Described herein are companion diagnostic methods and kits for use in combination with a therapy comprising administration of a TEC family kinase inhibitor. In some embodiments, the companion diagnostic methods provided involve protein occupancy assays for one or more inhibitors of the TEC kinase family. Accordingly, described herein are protein occupancy assays for kinase inhibitors of the TEC kinase family. Further described herein are protein occupancy assays for irreversible kinase inhibitors of the TEC kinase family. Further described herein are protein occupancy assays for reversible kinase inhibitors of the TEC kinase family. In some embodiments, the TEC kinase family inhibitor is an inhibitor of one or more kinases of the TEC kinase family. In some embodiments, the TEC kinase family inhibitor is an inhibitor of BTK, ITK, BMX, TXK, TEC, or any combination thereof. In some embodiments, the TEC kinase family inhibitor is an inhibitor of one or more kinases of the TEC kinase family and also is an inhibitor of one or more structurally homologous kinases. In some embodiments, the TEC kinase family inhibitor is an inhibitor of one or more kinases of the TEC kinase family and also is an inhibitor of one or more structurally homologous tyrosine kinases (e.g., a kinase that has a structurally homologous active site). In some embodiments, the TEC kinase family inhibitor is an inhibitor of one or more kinases of the TEC kinase family and also is an inhibitor of a kinase of the EGFR family. In some embodiments, the TEC kinase family inhibitor is an inhibitor of one or more kinases of the TEC kinase family and also is an inhibitor of HER1 (EGFR, ErbB1), HER2/c-neu (ErbB2), HER3 (ErbB3) and HER4 (ErbB4), or JAK3. In some embodiments, the TEC kinase family inhibitor is an inhibitor of one or more kinases of the TEC kinase family and also is an inhibitor of a SRC family tyrosine kinase. In some embodiments, the TEC kinase family inhibitor is an inhibitor of one or more kinases of the TEC kinase family and also is an inhibitor of B lymphoid kinase (BLK). Further described herein are exemplary reagents and probes for use in the protein occupancy assays provided.

Described herein in certain embodiments is protein occupancy assay that is an ELISA probe assay. In some embodiments, the ELISA probe assay is plate based electrochemiluminescent assay to determine the relative amount of a TEC family kinase that has not been bound by a TEC family kinase inhibitor. In some embodiments, TEC family kinase inhibitor is an irreversible TEC family kinase inhibitor. For example, in some embodiments, the TEC family kinase inhibitor binds to the active site of the TEC family kinase and forms a disulfide bond with a cysteine residue. In some embodiments, the assays involve binding a probe to TEC family kinases that have not been bound by the TEC family kinase inhibitor. In some embodiments, the probe comprises a TEC family kinase inhibitor attached to a detectable label (e.g., biotin) via a linker (e.g., a long chain linker). In some embodiments, the TEC family kinase inhibitor is a BTK inhibitor. In some embodiments, the TEC family kinase inhibitor is an irreversible BTK inhibitor. In some embodiments, the TEC family kinase inhibitor is ibrutinib. In some embodiments, the probe is Compound I-5 described herein, which consists of ibrutinib linked to biotin via a long chain linker. Labeling of samples with the probe allows for the detection of BTK not occupied by the TEC family kinase inhibitor. In some embodiments, the probe conjugated with the TEC family kinase (i.e. probe-bound kinase) is captured by a streptavidin coated plate. In some embodiments, excess un-conjugated probe competes with probe-bound kinase for binding to streptavidin.

Also described herein are methods for determining the efficacy of inhibitors of the TEC kinase family. Further described herein are methods for using the protein occupancy assays in the diagnosis, prognosis, and determination and modification of therapeutic regimens in the treatment of diseases associated with activation of one or more members of the TEC kinase family, including diseases wherein inhibition of one or more members of the TEC kinase family provides therapeutic benefit to a patient having the disease. In some embodiments, the patent is diagnosed as having a disease or disorder associated with aberrant activation of a TEC family kinase, such as, for example, cancer, an autoimmune disorder, and/or an inflammatory disease.

In one aspect, provided herein is a protein occupancy assay comprising a plate-based system. In some embodiments, the plate-based protein occupancy assay comprises an electrochemiluminescent assay.

Described herein are methods for determining the amount of TEC family kinases in a sample that have not been bound by a TEC family kinase inhibitor (e.g., active sites that are not occupied by the inhibitor). In some embodiments, the methods comprise determining the amount of BTK in a sample that have not been bound by a TEC family kinase inhibitor. In some embodiments, the TEC family kinase inhibitor is a BTK inhibitor. In some embodiments, the BTK inhibitor is ibrutinib. In some embodiments, the BTK inhibitor is AVL-292. In some embodiments, the BTK inhibitor is ONO-WG-307. In some embodiments, the methods comprise determining the amount of a TEC family kinase in a sample that is not bound to ibrutinib. In some embodiments, the methods comprise determining the amount of BTK in a sample that is not bound to ibrutinib. In some embodiments, the methods comprise determining the amount of ITK in a sample that is not bound to ibrutinib. In some embodiments, the methods comprise determining the amount of BMX in a sample that is not bound to ibrutinib. In some embodiments, the methods comprise determining the amount of TEC in a sample that is not bound to ibrutinib. In some embodiments, the methods comprise determining the amount of TXK in a sample that is not bound to ibrutinib. In some embodiments, the methods comprise determining the amount of BLK in a sample that is not bound to ibrutinib.

In some embodiments, the methods comprise determining the number of BTK kinase active sites in a sample that have not been bound by a TEC family kinase inhibitor. In some embodiments, the methods comprise determining the number of ITK kinase active sites in a sample that have not been bound by a TEC family kinase inhibitor. In some embodiments, the methods comprise determining the number of BMX active sites in a sample that have not been bound by a TEC family kinase inhibitor. In some embodiments, the methods comprise determining the number of TXK active sites in a sample that have not been bound by a TEC family kinase inhibitor. In some embodiments, the methods comprise determining the number of TEC active sites in a sample that have not been bound by a TEC family kinase inhibitor. In some embodiments, the methods comprise determining the number of BLK active sites in a sample that have not been bound by a TEC family kinase inhibitor. In some embodiments, the TEC family kinase inhibitor inhibits two or more members of the TEC kinase family. In some embodiments, the TEC family kinase inhibitor inhibits BTK, ITK, BMX, TXK, TEC or any combination thereof. In some embodiments, the TEC family kinase inhibitor inhibits an EGFR or SRC family tyrosine kinase.

In some embodiments, the methods comprise determining the number of BTK kinase active sites in a sample that have not been bound by ibrutinib. In some embodiments, the methods comprise determining the number of ITK kinase active sites in a sample that have not been bound by ibrutinib. In some embodiments, the methods comprise determining the number of BMX active sites in a sample that have not been bound by ibrutinib. In some embodiments, the methods comprise determining the number of TXK active sites in a sample that have not been bound by ibrutinib. In some embodiments, the methods comprise determining the number of TEC active sites in a sample that have not been bound by ibrutinib. In some embodiments, the methods comprise determining the number of BLK active sites in a sample that have not been bound by ibrutinib.

In some embodiments, the protein occupancy assay comprises contacting a sample with a probe, wherein the probe comprises a TEC family kinase inhibitor attached to a label via linker; and detecting a TEC family kinase bound to the probe (i.e. probe-bound kinase). In some embodiments, the probe is a derivative of ibrutinib, where ibrutinib is attached to a label via a linker. In some embodiments, the label is biotin or a derivative thereof. In some embodiments, the probe is selected from among probe of Formula (I), (II), or (III) as described herein. In some embodiments, the probe is selected from among probe compounds I-1, I-2, I-3, I-4, or I-5 as described herein. In some embodiments, the probe is probe compound I-5.

Disclosed herein in some embodiments are companion diagnostic kits for the detection of protein occupancy in a sample from a patient that has been administered a TEC family kinase inhibitor. In some embodiments, the kit comprise a probe that binds to the TEC family kinase not bound to the TEC family kinase inhibitor. In some embodiments, the probe comprises an inhibitor that binds to TEC family kinase (e.g., the probe is a derivative of a TEC family kinase inhibitor). In some embodiments, the inhibitor is attached to a label. In some embodiments, the probe further comprises a linker, wherein the linker is capable of attaching the label to the inhibitor. In some embodiments, the probe is a derivative of a TEC family kinase inhibitor. In some embodiments, the probe is a TEC family kinase inhibitor attached to a label via a linker. In some embodiments, the probe is a derivative of ibrutinib. In some embodiments, the probe consists of ibrutinib attached to a label via a linker. In some embodiments, the probe consists of ibrutinib attached to biotin via a linker. In some embodiments, the probe is a compound of Formula (I), (II) or (III). In some embodiments, the probe is compound I-1, I-2, I-3, I-4 or I-5. In some embodiments, the kit further comprises one or more solid supports.

Further disclosed herein are methods comprising: (a) contacting a sample comprising a TEC family kinase, or a homologous tyrosine kinase, with a probe; (b) detecting the amount of the probe-bound kinase; and (c) determining occupancy of the kinase based on the amount of the probe-bound kinase in the sample. In some embodiments, the method further comprises contacting the probe-bound kinase with a primary detection agent. In some embodiments, the method further comprises contacting the primary detection agent with a secondary detection agent.

Further disclosed herein are methods for determining a therapeutic regimen comprising: (a) contacting a sample comprising a TEC family kinase with a probe; (b) detecting the amount of a probe-bound kinase; (c) determining occupancy of a kinase based on the amount of the probe-bound kinase; and (d) determining a therapeutic regimen based on the occupancy of the kinase. In some embodiments, determining the therapeutic regimen comprises administering a TEC family kinase inhibitor. In some embodiments, determining the therapeutic regimen comprises modifying a therapeutic regimen with a TEC family kinase inhibitor. In some embodiments, modifying a therapeutic regimen comprises increasing, decreasing, initiating, or terminating a therapeutic regimen with a TEC family kinase inhibitor. In some embodiments, the therapeutic regimen with a TEC family kinase inhibitor is modified when the occupancy of the target increases. In some embodiments, the therapeutic regimen with a TEC family kinase inhibitor is modified when the occupancy of the target decreases. In some embodiments, the method further comprises contacting the probe-bound kinase with a primary detection agent. In some embodiments, the method further comprises contacting the primary detection agent with a secondary detection agent.

Further disclosed herein are methods for determining efficacy of a TEC family kinase inhibitor comprising: (a) contacting a sample comprising a TEC family kinase with a probe; (b) detecting the presence or absence of a probe-bound kinase; (c) determining occupancy of the kinase based on the amount of the probe-bound kinase; and (d) determining efficacy of the TEC family kinase inhibitor based on the occupancy of the kinase. In some embodiments, the TEC family kinase inhibitor is effective when the occupancy of the target is at least about 70%. In some embodiments, the TEC family kinase inhibitor is ineffective when the occupancy of the target is less than about 50%. In some embodiments, the method further comprises contacting the probe-bound kinase with a primary detection agent. In some embodiments, the method further comprises contacting the primary detection agent with a secondary detection agent.

Further disclosed herein are methods for identifying responders of a TEC family kinase inhibitor therapy comprising: (a) contacting a sample comprising a TEC family kinase with a probe, wherein the sample is from a subject having received at least one administration of the TEC family kinase inhibitor; (b) detecting the amount of the probe-bound kinase; (c) determining occupancy of the TEC family kinase based on the amount of the probe-bound kinase; and (d) identifying the subject as a TEC family kinase inhibitor responder or TEC family kinase inhibitor non-responder based on the occupancy of the kinase. In some embodiments, the subject is identified as a TEC family kinase inhibitor responder when the occupancy of the target is at least about 70%. In some embodiments, the subject is identified as a TEC family kinase inhibitor non-responder when the occupancy of the target is less than about 50%. In some embodiments, the method further comprises contacting the probe-bound kinase with a primary detection agent. In some embodiments, the method further comprises contacting the primary detection agent with a secondary detection agent.

Further disclosed herein are methods for determining TEC family kinase inhibitor resistance comprising: (a) contacting a sample comprising a TEC family kinase with a probe; (b) detecting the amount of the probe-bound kinase; (c) determining occupancy of the TEC family kinase based on the amount of the probe-bound kinase; and (d) determining TEC family kinase inhibitor resistance based on the occupancy of the TEC family kinase. In some embodiments, TEC family kinase inhibitor resistance is determined when the occupancy of the target is less than about 50%. In some embodiments, the method further comprises contacting the probe-bound kinase with a primary detection agent. In some embodiments, the method further comprises contacting the primary detection agent with a secondary detection agent.

Further disclosed herein are methods for validating a TEC family kinase inhibitor comprising: (a) contacting a sample comprising a TEC family kinase with a probe to form a probe-bound kinase; (b) detecting the amount of the probe-bound kinase; and (c) determining occupancy of the kinase by the TEC family kinase inhibitor based on the amount of the probe-bound target; and (d) validating the TEC family kinase inhibitor based on the occupancy of the TEC family kinase. In some embodiments, validating the TEC family kinase inhibitor comprises determining the efficacy of the TEC family kinase inhibitor on a TEC family kinase. In some embodiments, determining occupancy of the TEC family kinase by the TEC family kinase inhibitor comprises quantifying the amount of probe-bound kinases. In some embodiments, the drug is effective when the occupancy of the TEC family kinase is at least about 70%. In some embodiments, the method further comprises contacting the probe-bound kinase with a primary detection agent. In some embodiments, the method further comprises contacting the primary detection agent with a secondary detection agent.

Further disclosed herein are methods method for identifying TEC family kinase modulators that bind to TEC family kinases comprising: (a) contacting a sample comprising a TEC family kinase with a probe; (b) detecting the presence or absence of a probe-bound kinase; and (c) identifying TEC family kinase modulators based on the amount of the probe-bound kinase. In some embodiments, the method further comprises contacting the probe-bound kinase with a primary detection agent. In some embodiments, the method further comprises contacting the primary detection agent with a secondary detection agent. In some embodiments, the sample is a sample that pre-treated with the putative TEC family kinase modulator. In some embodiments, the sample is pre-treated in vitro. In some embodiments, the sample is a sample from a subject (e.g., a patient) that has been administered the putative TEC family kinase modulator.

Further disclosed herein are methods method for identifying TEC family kinase inhibitor comprising: (a) contacting a sample comprising a TEC family kinase with a probe; (b) detecting the presence or absence of a probe-bound kinase; and (c) identifying TEC family kinase inhibitor based on the amount of the probe-bound kinase. In some embodiments, the method further comprises contacting the probe-bound kinase with a primary detection agent. In some embodiments, the method further comprises contacting the primary detection agent with a secondary detection agent. In some embodiments, the sample is a sample that pre-treated with the putative TEC family kinase inhibitor. In some embodiments, the sample is pre-treated in vitro. In some embodiments, the sample is a sample from a subject (e.g., a patient) that has been administered the putative TEC family kinase inhibitor.

In some embodiments, the methods, kits or compositions disclosed herein comprise a TEC family kinase inhibitor. In some embodiments, the inhibitor is an irreversible TEC family kinase inhibitor. In some embodiments, the inhibitor covalently binds to a TEC family kinase. In some embodiments, the inhibitor binds to a cysteine residue of a TEC family kinase. In some embodiments, the inhibitor is a small molecule, polypeptide, antibody, or nucleic acid. In some embodiments, the TEC family kinase inhibitor is an inhibitor of a Bruton's tyrosine kinase (BTK). In some embodiments, the inhibitor of a Bruton's tyrosine kinase (BTK) is ibrutinib. In some embodiments, the inhibitor of a Bruton's tyrosine kinase (BTK) is AVL-292, AVL-291, AVL-101, CNX-774, ONO-WG-307. In some embodiments, the TEC family kinase inhibitor is an inhibitor of an ITK. In some embodiments, the TEC family kinase inhibitor is an inhibitor of a TEC kinase. In some embodiments, the TEC family kinase inhibitor is an inhibitor of a BMX kinase. In some embodiments, the TEC family kinase inhibitor is an inhibitor of a BLK. In some embodiments, the TEC family kinase inhibitor is an inhibitor of a kinase selected from HER1, HER2, HER3, HER4 and JAK3.

In some embodiments, the methods, kits or compositions disclosed herein comprise a target kinase. In some embodiments, the target kinase is a TEC family kinase. In some embodiments, the kinase is a Bruton's tyrosine kinase (BTK). In some embodiments, the kinase is an ITK. In some embodiments, the kinase is a BLK. In some embodiments, the kinase is a TEC kinase. In some embodiments, the kinase is a TXK. In some embodiments, the kinase is a BMX kinase. In some embodiments, the kinase is ITK. In some embodiments, the kinase is HER1, HER2, HER3, HER4, or JAK3.

In some embodiments, the methods, kits or compositions disclosed herein comprise one or more solid supports. In some embodiments, the one or more solid supports is a plate. In some embodiments, the one or more solid supports is a bead or a plurality of beads. In some embodiments, the kit comprises two or more solid supports. In some embodiments, the two or more solid supports comprise (a) a plate; and (b) a bead or a plurality of beads. In some embodiments, the plate is a microplate. In some embodiments, the microplate is a streptavidin-coated microplate. In some embodiments, the microplate is a MSD microplate. In some embodiments, the bead is a streptavidin bead. In some embodiments, the bead is a magnetic bead. In some embodiments, the solid support is coated to form a coated solid support. In some embodiments, the coated solid support is coated with streptavidin. In some embodiments, the coated solid support is coated with an antibody. In some embodiments, the coated solid support is capable of capturing probe. In some embodiments, the coated solid support is capable of capturing the label. In some embodiments, the coated solid support is capable of capturing a target (e.g., a TEC family kinase).

In some embodiments, the methods disclosed herein further comprise contacting the probe-bound target with a primary detection agent. In some embodiments, the primary detection agent comprises an antibody, a bead, a dye, a label, a tag, a fluorophore, or any combination thereof. In some embodiments, the primary detection agent comprises an antibody. In some embodiments, the antibody is an anti-BTK antibody. In some embodiments, the primary detection agent is conjugated to a tag. In some embodiments, the primary detection agent is conjugated to an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the primary detection agent is conjugated to a SULFO tag. In some embodiments, the primary detection agent is a bead.

In some embodiments, the methods disclosed herein further comprise contacting the primary detection agent with a secondary detection agent. In some embodiments, the secondary detection agent comprises an antibody, a bead, a dye, a label, a tag, a fluorophore, or any combination thereof. In some embodiments, the secondary detection agent comprises an antibody. In some embodiments, the antibody is an anti-IgG antibody. In some embodiments, the antibody is an anti-IgA antibody. In some embodiments, the secondary detection agent is conjugated to a tag. In some embodiments, the secondary detection agent is conjugated to an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the secondary detection agent is conjugated to a SULFO tag. In some embodiments, the secondary detection agent is a bead.

In some embodiments, the methods, kits or compositions disclosed herein comprise a primary detection agent. In some embodiments, the methods, kits or compositions disclosed herein comprise a secondary detection agent. In some embodiments, the detection agent comprises an antibody, a bead, a dye, a label, a tag, a fluorophore, or any combination thereof. In some embodiments, the detection agent detection agent comprises an antibody. In some embodiments, the detection agent is conjugated to a tag. In some embodiments, the detection agent is conjugated to an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the detection agent conjugated to a SULFO tag. In some embodiments, the detection agent is a bead. In some embodiments, the detection agent is conjugated to an enzyme. In some embodiments, the antibody is conjugated to horseradish peroxidase (HRP) or alkaline phosphatase (AP). In some embodiments, the antibody is an anti-BTK antibody. In some embodiments, the antibody targets a TEC family kinase. In some embodiments, the antibody is an anti-ITK antibody. In some embodiments, the antibody is an anti-TEC antibody. In some embodiments, the antibody is an anti-BMX antibody. In some embodiments, the antibody is an anti-BLK antibody. In some embodiments, the antibody is an anti-HER1 antibody, anti-HER2 antibody, anti-HER3 antibody, or anti-HER4 antibody.

In some embodiments, the methods, kits or compositions disclosed herein comprise a label. In some embodiments, the label is biotin. In some embodiments, the label is a fluorophore.

In some embodiments, the methods disclosed herein further comprise capturing the target kinase. In some embodiments, the methods disclosed herein further comprise capturing the probe-bound target kinase. In some embodiments, the target is captured by an antibody. In some embodiments, the antibody is an anti-target antibody. In some embodiments, the probe-bound target is captured by a bead.

In some embodiments, the methods, kits or compositions disclosed herein comprise an antibody. In some embodiments, the antibody is attached to a solid support. In some embodiments, the bead is attached to a solid support. In some embodiments, the solid support is a microplate. In some embodiments, the microplate is a MSD microplate. In some embodiments, the solid support is a bead.

In some embodiments, the methods, kits, and compositions disclosed herein comprise a bead. In some embodiments, the bead is a streptavidin bead. In some embodiments, the bead is a magnetic bead. In some embodiments, the bead is a coated bead. In some embodiments, the bead is a coated streptavidin bead. In some embodiments, the coated bead is coated with a tag. In some embodiments, the tag is an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the bead is a SULFO tag streptavidin bead. In some embodiments, the bead is a SULFO tag bead. In some embodiments, the bead interacts with the probe. In some embodiments, the probe comprises a label. In some embodiments, the bead interacts with the label. In some embodiments, the label comprises biotin. In some embodiments, the bead forms a conjugate with the probe-bound target. In some embodiments, the bead binds to the probe.

In some embodiments, the methods disclosed herein comprise detecting the presence or absence of the probe-bound target or a portion thereof. In some embodiments, detecting the presence or absence of the probe-bound target comprises detecting the probe-bound target or a portion thereof. In some embodiments, detecting the presence or absence of the probe-bound target comprises detecting the bead or a portion thereof. In some embodiments, detecting the presence or absence of the probe-bound target comprises detecting the coated bead. In some embodiments, detecting the presence or absence of the probe-bound target comprises detecting a electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, detecting the presence or absence of the probe-bound target comprises detecting a SULFO tag. In some embodiments, detecting the presence or absence of the probe-bound target comprises luminescence. In some embodiments, detecting the presence or absence of the probe-bound target comprises electrochemiluminescence. In some embodiments, the probe-bound target is an unoccupied target. In some embodiments, the probe-bound target is a drug-occupied target.

In some embodiments, the methods disclosed herein further comprise purification of the probe-bound target. In some embodiments, purification of the probe-bound target comprises magnetic separation of probe-bound targets from non-probe-bound targets. In some embodiments, the sample is a pre-treated sample, wherein the pre-treated sample is contacted with a TEC family kinase inhibitor prior to contact with the probe. In some embodiments, the sample is a non-treated sample, wherein the sample is not contacted with a TEC family kinase inhibitor prior to contact with the label. In some embodiments, the sample is a sample from a patient that has been administered a TEC family kinase inhibitor. In some embodiments, the sample is a control sample from a patient that has not been administered a TEC family kinase inhibitor. In some embodiments, the sample is a whole blood sample, peripheral blood sample, lymph sample, tissue sample, tumor biopsy sample, or bone marrow sample. In some embodiments, the sample is a sample containing one or more cell types, or a lysate thereof, derived from a whole blood sample, peripheral blood sample, lymph sample, tissue sample, tumor biopsy sample, or bone marrow sample.

In some embodiments, the methods, kits, and compositions disclosed herein comprise a probe. In some embodiments, the probe comprises an inhibitor. In some embodiments, the inhibitor binds to a target. In some embodiments, the inhibitor is an irreversible inhibitor. In some embodiments, the inhibitor covalently binds to a target. In some embodiments, the inhibitor binds to a cysteine residue of a target. In some embodiments, the inhibitor is a small molecule, polypeptide, antibody, or nucleic acid. In some embodiments, the inhibitor is an inhibitor of a TEC family kinase. In some embodiments, the inhibitor is an inhibitor of a Bruton's tyrosine kinase (BTK). In some embodiments, the inhibitor binds to a cysteine residue of a Bruton's tyrosine kinase (BTK). In some embodiments, the inhibitor binds to a cysteine 481 of a Bruton's tyrosine kinase (BTK). In some embodiments, the inhibitor of a Bruton's tyrosine kinase (BTK) is ibrutinib. In some embodiments, the inhibitor of a Bruton's tyrosine kinase (BTK) is AVL-292, AVL-291 AVL-101, CNX-774, ONO-WG-307. In some embodiments, the agent is an inhibitor of an ITK. In some embodiments, the agent is an inhibitor of a TEC kinase. In some embodiments, the agent is an inhibitor of a BMX kinase. In some embodiments, the agent is an inhibitor of a BLK. In some embodiments, the agent is an inhibitor of a kinase selected from HER1, HER2, HER3, HER4 and JAK3.

In some embodiments, the methods, kits, and compositions disclosed herein comprise a target. In some embodiments, the target is a kinase. In some embodiments, the kinase is a Bruton's tyrosine kinase (BTK). In some embodiments, the kinase is ITK, BLK, TEC, TXK, or BMX. In some embodiments, the kinase is HER1, HER2, HER3, HER4, or JAK3. In some embodiments, the target is a protein.

In some embodiments, the methods, kits, and compositions disclosed herein comprise a sample. In some embodiments, the sample is from a subject suffering from a cancer. In some embodiments, the cancer is a sarcoma. In some embodiments, the cancer is a carcinoma. In some embodiments, the cancer is a lymphoma. In some embodiments, the lymphoma is a Hodgkin's lymphoma. In some embodiments, the lymphoma is a non-Hodgkin's lymphoma (NHL). In some embodiments, the cancer is a leukemia. In some embodiments, the cancer is a chronic lymphocytic leukemia. In some embodiments, the cancer is a small lymphocytic leukemia. In some embodiments, the cancer is Waldenstrom's macroglobulinemia. In some embodiments, the cancer is a follicular lymphoma. In some embodiments, the cancer is a mantle cell lymphoma. In some embodiments, the cancer is a diffuse large B-cell lymphoma. In some embodiments, the cancer is a multiple myeloma. In some embodiments, the cancer is a solid tumor. In some embodiments, the sample is from a subject suffering from a an autoimmune or inflammatory disorder.

Described herein, in particular embodiments, are kits for determining drug target occupancy in a patient receiving a TEC family kinase inhibitor therapy, comprising a probe having the structure of Formula (II) comprising:

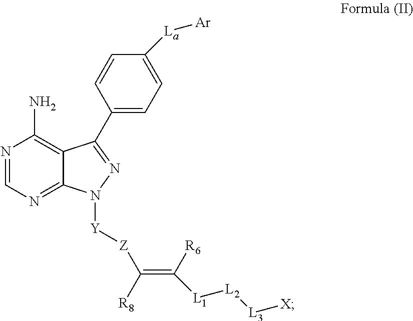

wherein:
La is CH2, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), S(O)n, OS(O)n, NHS(O)n, where n is 1 or 2;
R6 and R8 are independently selected from H, optionally substituted alkyl, or optionally substituted heteroalkyl;
L1 is optionally substituted alkyl or optionally substituted heteroalkyl;
L2 is a bond, optionally substituted heterocycloalkyl, or —N(H)C(O)(CH2)mC(O)N(H), where m is 2-6;

L3 is optionally substituted alkyl or optionally substituted heteroalkyl; and X is a detectable label, wherein the probe binds to a TEC family kinase. In some embodiments, X is:

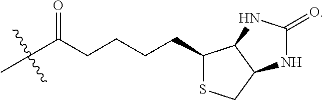 [SMILES: *C(=O)CCCC[C@@H]1SC[C@@H]2NC(=O)N[C@@H]21]

In some embodiments, the probe has the structure of:

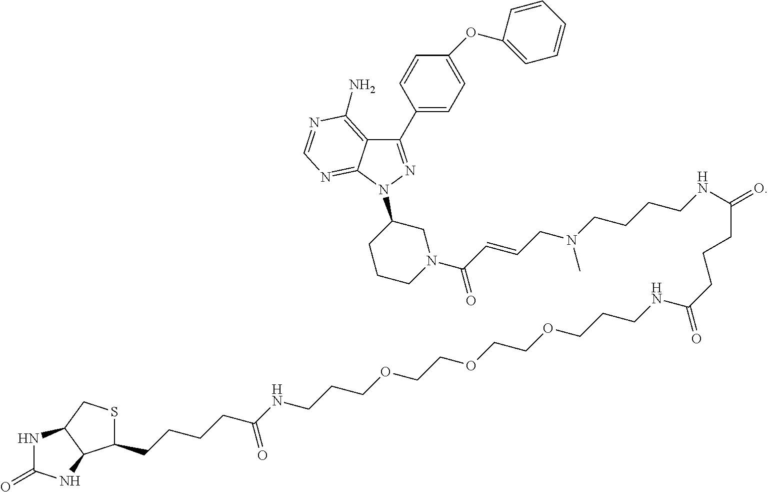 [SMILES: CN(C/C=C/C(=O)N1CCC[C@@H](n2nc(-c3ccc(Oc4ccccc4)cc3)c3c(N)ncnc32)C1)CCCCNC(=O)CCCC(=O)NCCCOCCOCCOCCCNC(=O)CCCC[C@@H]1SC[C@@H]2NC(=O)N[C@@H]21]

In some embodiments, the TEC family kinase is Bruton's tyrosine kinase (BTK), ITK, TEC, BMX, or TXK. In some embodiments, the probe binds to BLK, HER1, HER2, HER3, HER4 or JAK3. In some embodiments, the kits further comprise one or more solid supports. In some embodiments, the one or more solid supports are selected from among a plate, a microplate, a bead or a plurality of beads. In some embodiments, the solid support is coated with a capture agent to form a coated solid support, wherein the capture agent binds to the probe. In some embodiments, the capture agent is streptavidin or an antibody. In some embodiments, the kits further comprise a primary detection agent, and optionally, a secondary detection agent that binds to the primary detection agent. In some embodiments, the primary detection agent or secondary detection agent comprises an antibody, a bead, a dye, a label, a tag, a fluorophore, or any combination thereof. In some embodiments, the primary detection agent is an antibody that is an anti-BTK antibody, an anti-ITK antibody, an anti-TEC antibody, an anti-TXK antibody, an anti-BMX antibody, or an anti-BLK antibody. In some embodiments, the primary detection agent is an antibody that is an anti-HER1 antibody, an anti-HER2 antibody, an anti-HER3 antibody, or an anti-HER4 antibody. In some embodiments, the primary or secondary detection agent is conjugated to an electrochemiluminescent tag. In some embodiments, chemiluminescent tag is a Ruthenium (II) tris-bipyridine, N-hydroxysuccinimide tag. In some embodiments, the TEC kinase inhibitor therapy is an irreversible TEC kinase inhibitor. In some embodiments, the TEC kinase inhibitor therapy is an irreversible BTK inhibitor. In some embodiments, the TEC kinase inhibitor therapy is ibrutinib.

Described herein, in certain embodiments are methods for determining drug target occupancy in a patient receiving a TEC family kinase inhibitor therapy, comprising: (a) contacting a sample comprising a TEC family kinase with a probe to form a probe-bound kinase, wherein the sample is obtained from the patient following administration of at least one dose of an irreversible TEC family kinase inhibitor; (b) detecting the amount of probe-bound kinase in the sample; and (c) determining target occupancy of the TEC family kinase based on the amount of probe-bound kinase detected in the sample, wherein the probe has the structure of Formula (II) comprising:

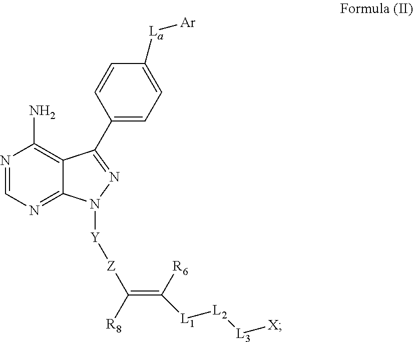

wherein:
La is CH2, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), S(O)n, OS(O)n, NHS(O)n, where n is 1 or 2;
R6 and R8 are independently selected from H, optionally substituted alkyl, or optionally substituted heteroalkyl;
L1 is optionally substituted alkyl or optionally substituted heteroalkyl;
L2 is a bond, optionally substituted heterocycloalkyl, or —N(H)C(O)(CH2)mC(O)N(H), where m is 2-6;
L3 is optionally substituted alkyl or optionally substituted heteroalkyl; and X is a detectable label, wherein the probe binds to a TEC family kinase. In some embodiments, X is:

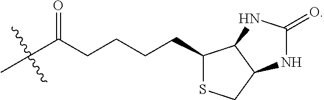 [SMILES: *C(=O)CCCC[C@@H]1SC[C@@H]2NC(=O)N[C@@H]21]

In some embodiments, the probe has the structure of:

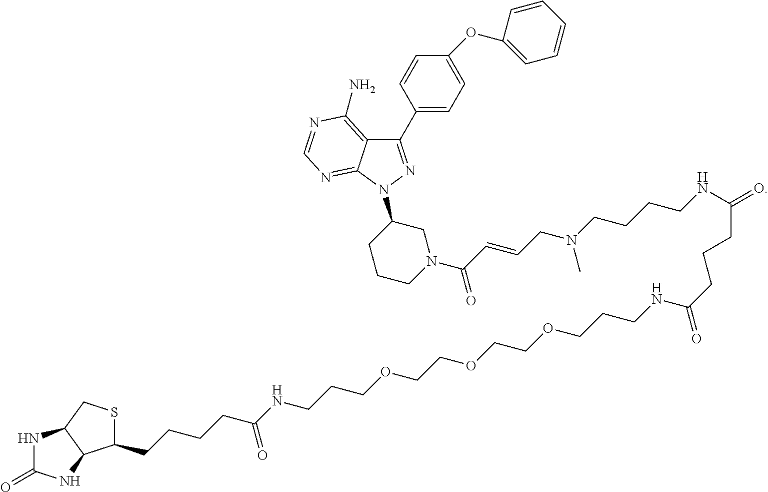 [SMILES: CN(C/C=C/C(=O)N1CCC[C@@H](n2nc(-c3ccc(Oc4ccccc4)cc3)c3c(N)ncnc32)C1)CCCCNC(=O)CCCC(=O)NCCCOCCOCCOCCCNC(=O)CCCC[C@@H]1SC[C@@H]2NC(=O)N[C@@H]21]

In some embodiments, determining target occupancy comprises i) determining the number of binding sites not bound to the TEC family kinase inhibitor based on the amount of probe-bound kinase detected in the sample and ii) comparing said number to the total amount of active TEC family kinases in the sample. In some embodiments, the control is the amount of probe-bound kinase that is present when the method is performed on an untreated sample. In some embodiments, the methods further comprise determining or modifying a therapeutic regimen based on the target occupancy of the TEC family kinase. Described herein are methods for monitoring drug target occupancy in a patient receiving a TEC family kinase inhibitor therapy, comprising performing the methods provided herein for determining protein occupancy of the kinase at two or more time points over the course of the therapy. In some embodiments, the methods further comprise modifying a therapeutic regimen if the target occupancy increases or decreases over the course of the therapy. In some embodiments, the methods further comprise: i) increasing the dosage or frequency of administration of the TEC family kinase inhibitor if the target occupancy is less than about 50%, ii) decreasing the dosage or frequency of administration of the TEC family kinase inhibitor if the target occupancy is above at least about 70%, iii) maintaining the same therapeutic regimen of the TEC family kinase inhibitor or iv) discontinuing the therapeutic regimen. In some embodiments, the dosage of the TEC family kinase inhibitor is increased if the target occupancy is less than about 50%. In some embodiments, the dosage of the TEC family kinase inhibitor is decreased if the target occupancy is above at least about 70%. In some embodiments, the dosage of the TEC family kinase inhibitor is maintained if the target occupancy is above at least about 70%. In some embodiments, the frequency of administration of the TEC family kinase inhibitor is increased if the target occupancy is less than about 50%. In some embodiments, the frequency of administration of the TEC family kinase inhibitor is decreased if the target occupancy is above at least about 70%. In some embodiments, the frequency of administration of the TEC family kinase inhibitor is maintained if the target occupancy is above at least about 70%. In some embodiments, the methods further comprise determining the efficacy of the TEC family kinase inhibitor therapy based on the target occupancy. In some embodiments, the TEC family kinase inhibitor is effective when the occupancy of the TEC family kinase is at least about 70%. In some embodiments, the TEC family kinase inhibitor is ineffective when the occupancy of the TEC family kinase is less than about 50%. In some embodiments, the TEC family kinase inhibitor an inhibitor of a Bruton's tyrosine kinase (BTK). In some embodiments, the TEC family kinase inhibitor is ibrutinib, AVL-292, AVL-291, AVL-101, CNX-774, or ONO-WG-307. In some embodiments, the TEC family kinase inhibitor is ibrutinib. In some embodiments, the at least one dosage of ibrutinib is about 10 mg to about 2000 mg, such as, for example, 140 mg, 420 mg, 560 mg or 840 mg. In some embodiments, where protein occupancy is monitored over the course of a therapy, the patient is receiving a daily dosage of ibrutinib is about 10 mg per day to about 2000 mg per day, such as, for example, a daily dosage of about 140 mg per day, 420 mg per day, 560 mg per day or 840 mg per day. In a particular embodiment, the patient is receiving a maintenance dosage of ibrutinib of about 420 mg per day. In some embodiments, the methods further comprise capturing probe-bound kinase with a capture agent. In some embodiments, the capture target is streptavidin or an antibody. In some embodiments, the capture target is attached to a solid support. In some embodiments, the solid support is a plate, a microplate, a bead or a plurality of beads. In some embodiments, the methods further comprise contacting the probe-bound kinase with a primary detection agent, and optionally, a secondary detection agent that binds to the primary detection agent. In some embodiments, the primary detection agent or secondary detection agent comprises an antibody, a bead, a dye, a label, a tag, a fluorophore, or any combination thereof. In some embodiments, the primary detection agent is an antibody binds to a TEC family kinase. In some embodiments, the primary detection agent is an antibody that is an anti-BTK antibody, an anti-ITK antibody, an anti-TXK antibody, anti-TEC antibody, anti-BMX antibody, or anti-BLK antibody. In some embodiments, the primary detection agent is an antibody that is an anti-HER1 antibody, anti-HER2 antibody, anti-HER3 antibody, or anti-HER4 antibody. In some embodiments, the methods further comprise contacting the primary detection agent with a secondary detection agent. In some embodiments, the secondary detection agent comprises an antibody, a bead, a dye, a label, a tag, a fluorophore, or any combination thereof. In some embodiments, the primary or secondary detection agent is conjugated to a chemiluminescent tag. In some embodiments, the chemiluminescent tag is a Ruthenium (II) tris-bipyridine, N-hydroxysuccinimide tag. In some embodiments, the patient is suffering from a cancer, an autoimmune disease or an inflammatory disorder. In some embodiments, the cancer is a sarcoma, carcinoma, myeloma, leukemia or a lymphoma. In some embodiments, the cancer is Hodgkin's lymphoma or a non-Hodgkin's lymphoma (NHL). In some embodiments, the cancer is chronic lymphocytic leukemia (CLL), small lymphocytic lymphoma (SLL), mantle cell leukemia (MCL), follicular lymphoma (FL), diffuse large B-cell lymphoma (DLBCL), Waldenström macroglobulinemia or multiple myeloma (MM). In some embodiments, the sample is a blood sample, a lymph sample or tumor biopsy sample.

### INCORPORATION BY REFERENCE

All publications and patent applications mentioned in this specification are herein incorporated by reference in their entireties to the same extent as if each individual publication or patent application was specifically and individually indicated to be incorporated by reference. The publications discussed herein are provided solely for their disclosure prior to the filing date of the present application. Nothing herein is to be construed as an admission that the inventors described herein are not entitled to antedate such disclosure by virtue of prior invention or for any other reason.

### BRIEF DESCRIPTION OF THE DRAWINGS

The skilled artisan will understand that the drawings described below are for illustration purposes only. The drawings are not intended to limit the scope of the present teachings in any way.

FIG. 1 illustrates an overview of the Protein Occupancy Assay

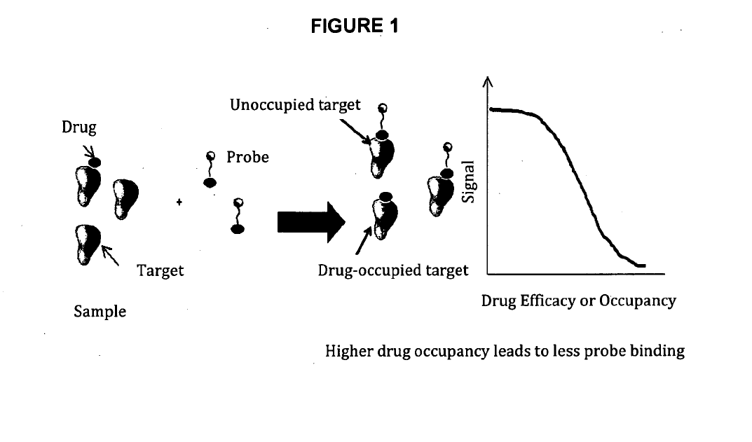

FIG. 2 illustrates components of a Protein Occupancy Assay Kit

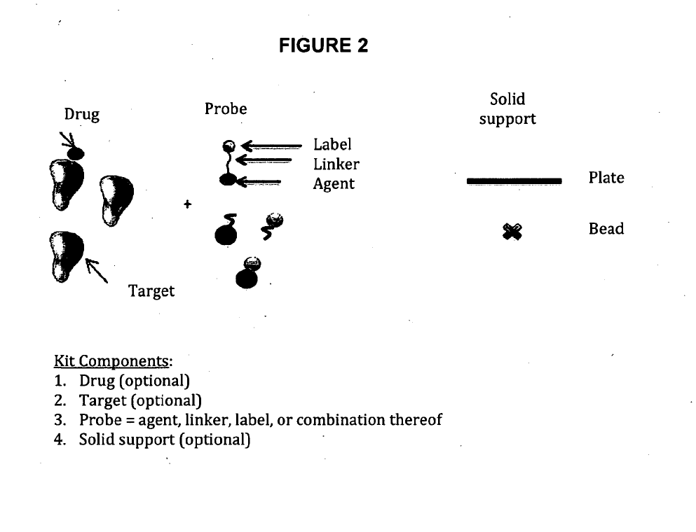

FIG. 3 illustrates an overview of a Protein occupancy assay method for the detection of drug-bound targets

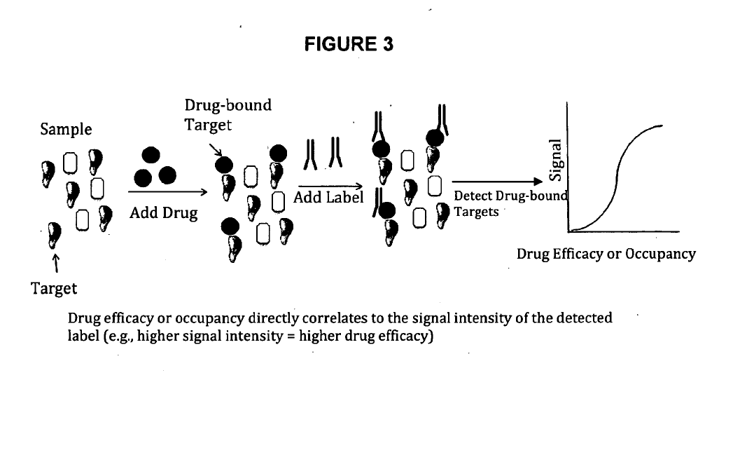

FIG. 4 illustrates an overview of a Protein occupancy assay for the detection of unoccupied targets

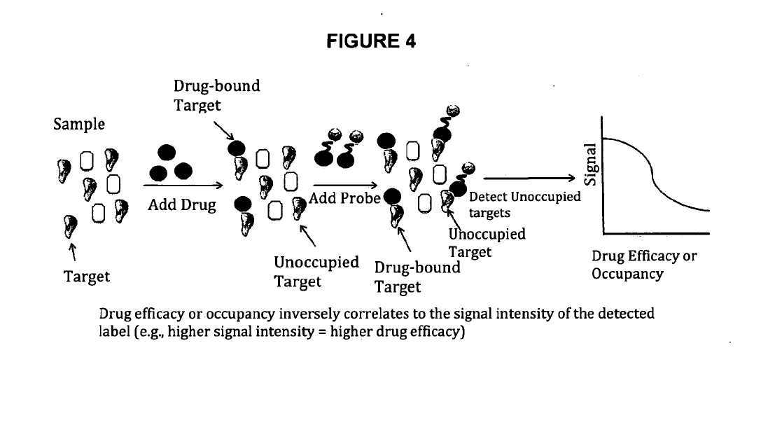

FIG. 5 illustrates an overview of a Plate-based Protein occupancy assay for the detection of drug-bound targets

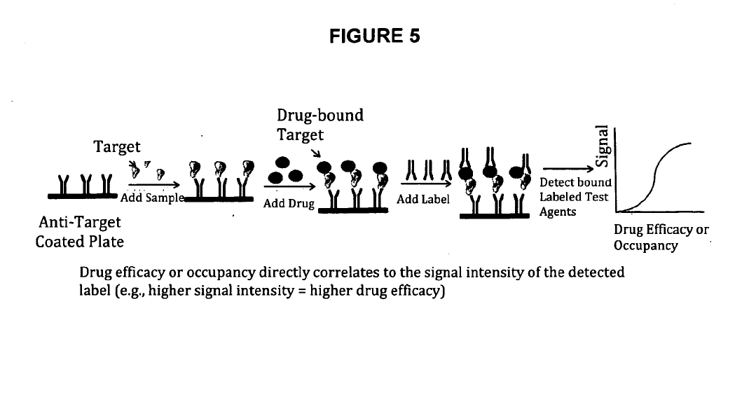

FIG. 6 illustrates an overview of a Plate-based Protein occupancy assay for the detection of probe-bound targets

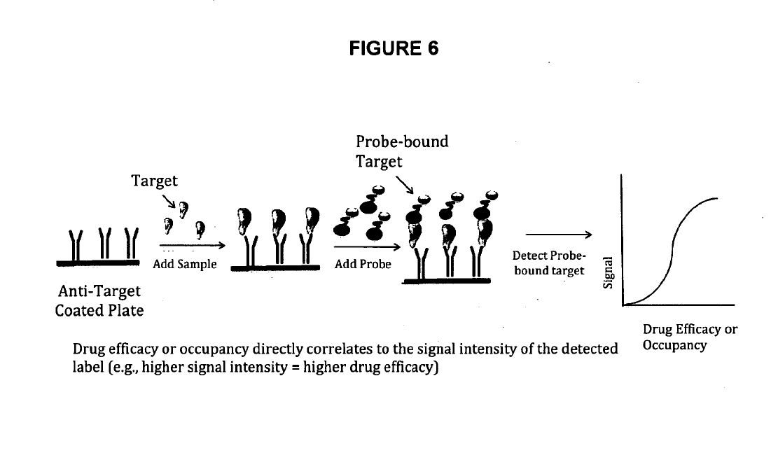

FIG. 7 illustrates an overview of a Plate-based protein occupancy assay for the detection of drug-bound targets

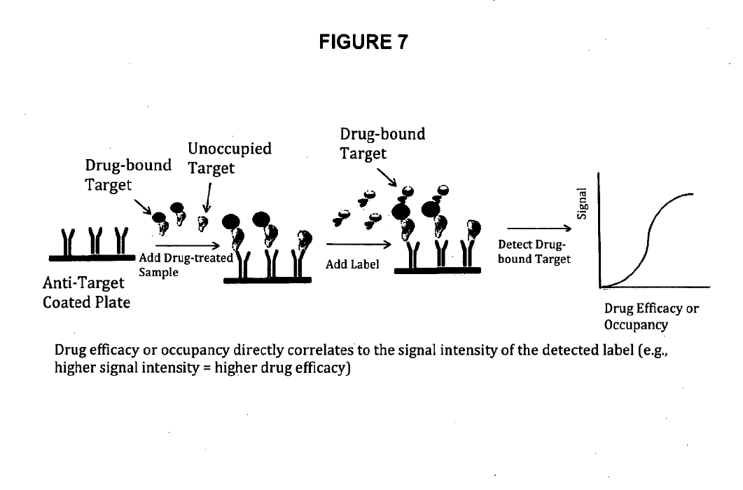

FIG. 8 illustrates an overview of a Plate-based protein occupancy assay for the detection of unoccupied targets

FIG. 9 illustrates an overview of a Probe-coated plate-based protein occupancy for the detection of probe-bound targets

FIG. 10 illustrates an overview of a Probe-coated plate-based protein occupancy assay for the detection of unoccupied targets

FIG. 11 illustrates exemplary BTK occupancy assay formats. FIG. 11A presents an illustrative overview of the streptavidin detection method. FIG. 11B presents an illustrative overview of the streptavidin capture method.

FIG. 12 illustrates a Streptavidin detection BTK occupancy assay. FIG. 12A presents an illustrative plate layout. FIG. 12B presents illustrative data showing the results for the streptavidin detection method

FIG. 13 presents illustrative results for a streptavidin detection BTK occupancy assay using two different BTK capture antibodies.

FIG. 14 illustrates a Streptavidin-capture BTK occupancy assay. FIG. 14A presents an overview of the Streptavidin-capture method. FIG. 14B presents an illustrative plate layout. FIG. 14C presents illustrative data showing the results for the streptavidin-capture method

FIG. 15 presents illustrative results for a streptavidin-capture BTK occupancy assay using two different BTK detection antibodies.

FIG. 16 illustrates a comparison of streptavidin detection and streptavidin capture methods

FIG. 17 presents illustrative results for a probe optimization experiment for a streptavidin capture BTK occupancy assay.

FIG. 18 illustrates the results for a titration experiment for a streptavidin capture BTK occupancy assay.

FIG. 19 illustrates the results for a titration experiment for a streptavidin capture BTK occupancy assay.

FIG. 20 illustrates a SI2400 MSD SECTOR IMAGER plate.

FIG. 21 illustrates exemplary probe compounds I-1, I-2, I-3, I-4 and I-5.

FIG. 22 presents illustrative results for exemplary probe compounds I-1, I-2, I-3, I-4 and I-5 for a probe optimization experiment for a streptavidin capture BTK occupancy assay.

FIG. 23 illustrates raw signal data for various capture antibody/detection antibody pairs tested for quantifying total BLK. Top, MSD high bind plate. Bottom, MSD standard plate.

FIG. 24 illustrates signal to background ratios for the different capture antibody/detection antibody pairs tested for quantifying total BLK. Top, MSD high bind plate. Bottom, MSD standard plate.

FIG. 25 illustrates raw signal data for dose titration of the capture antibody/detection antibody pairs tested for quantifying total BLK.

FIG. 26 illustrates signal to background ratios for the dose titration of the capture antibody/detection antibody pairs tested for quantifying total BLK.

FIG. 27 illustrates a plot of the signal values for recombinant BLK protein using 1 μg/ml capture antibody and 0.5 μg/ml detection antibody.

FIG. 28 illustrates the results for a probe titration experiment for a streptavidin capture BLK occupancy assay (A) and ITK occupancy assay (B).

FIG. 29 illustrates the results for a drug titration experiment for a streptavidin capture ITK occupancy assay (A) and % ITK inhibition (B).

FIG. 30 illustrates the results for a drug titration experiment for a streptavidin capture ITK occupancy assay using PBMC lysates. Results are expressed as % ITK inhibition.

### DETAILED DESCRIPTION OF THE INVENTION

Described herein are companion diagnostic methods and kits for use in combination with a therapy comprising administration of a TEC family kinase inhibitor. In some embodiments, the companion diagnostic methods provided involve protein occupancy assays for one or more inhibitors of the TEC kinase family. Accordingly, described herein are protein occupancy assays for kinase inhibitors of the TEC kinase family. Further described herein are protein occupancy assays for irreversible kinase inhibitors of the TEC kinase family. Further described herein are protein occupancy assays for reversible kinase inhibitors of the TEC kinase family. In some embodiments, the TEC kinase family inhibitor is an inhibitor of one or more kinases of the TEC kinase family. In some embodiments, the TEC kinase family inhibitor is an inhibitor of BTK, ITK, BMX, TXK, TEC, or any combination thereof. In some embodiments, the TEC kinase family inhibitor is an inhibitor of one or more kinases of the TEC kinase family and also is an inhibitor of one or more structurally homologous kinases. In some embodiments, the TEC kinase family inhibitor is an inhibitor of one or more kinases of the TEC kinase family and also is an inhibitor of one or more structurally homologous tyrosine kinases. In some embodiments, the TEC kinase family inhibitor is an inhibitor of one or more kinases of the TEC kinase family and also is an inhibitor of a kinase of the EGFR family. In some embodiments, the TEC kinase family inhibitor is an inhibitor of one or more kinases of the TEC kinase family and also is an inhibitor of HER1 (EGFR, ErbB1), HER2/c-neu (ErbB2), HER3 (ErbB3) and HER4 (ErbB4), or JAK3. In some embodiments, the TEC kinase family inhibitor is an inhibitor of one or more kinases of the TEC kinase family and also is an inhibitor of a SRC family tyrosine kinase. In some embodiments, the TEC kinase family inhibitor is an inhibitor of one or more kinases of the TEC kinase family and also is an inhibitor of B lymphoid kinase (BLK). Further described herein are exemplary reagents and probes for use in the protein occupancy assays provided.

Described herein in certain embodiments is protein occupancy assay that is an ELISA probe assay. In some embodiments, the ELISA probe assay is plate based electrochemiluminescent assay to determine the relative amount of a TEC family kinase that has not been bound by a TEC family kinase inhibitor. In some embodiments, TEC family kinase inhibitor is an irreversible TEC family kinase inhibitor. For example, in some embodiments, the TEC family kinase inhibitor binds to the active site of the TEC family kinase and forms a disulfide bond with a cysteine residue. In some embodiments, the assays involves binding an activity probe to free TEC family kinases that have not been bound by the TEC family kinase inhibitor. In some embodiments, the activity probe comprises a TEC family kinase inhibitor attached to a detectable label (e.g., biotin) via a linker (e.g., a long chain linker). In some embodiments, the TEC family kinase inhibitor a BTK inhibitor. In some embodiments, the TEC family kinase inhibitor is an irreversible BTK inhibitor. In some embodiments, the TEC family kinase inhibitor is ibrutinib. In some embodiments, the probe is Compound I-5, which consists of ibrutinib linked to biotin via a long chain linker. Labeling of samples with the probe allows for the detection of BTK not occupied by drug. In some embodiments, the probe conjugated with the TEC family kinase is captured by a streptavidin coated plate. In some embodiments, excess un-conjugated probe competes with probe labeled BTK for binding to streptavidin.

Also described herein are methods for determining the efficacy of inhibitors of the TEC kinase family. Further described herein are methods for using the protein occupancy assays in the diagnosis, prognosis, and determination and modification of therapeutic regimens in the treatment of diseases associated with activation of one or more members of the TEC kinase family, including diseases wherein inhibition of one or more members of the TEC kinase family provides therapeutic benefit to a patient having the disease. In some embodiments, the patent is diagnosed as having a disease or disorder associated with aberrant activation of a TEC family kinase, such as, for example, cancer, an autoimmune disorder, and/or an inflammatory disease.

Further disclosed herein are diagnostic assays for diagnosing, prognosing, and monitoring a disease or condition benefitting from treatment with a TEC family kinase inhibitor. Also disclosed herein are diagnostic assays for identifying responders to TEC family kinase inhibitor therapy, determining therapeutic regimens, and detecting resistance to TEC family kinase inhibitor therapy.

### CERTAIN TERMINOLOGY

Unless defined otherwise, all technical and scientific terms used herein have the same meaning as is commonly understood by one of skill in the art to which the claimed subject matter belongs. In the event that there is a plurality of definitions for terms herein, those in the section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Unless stated otherwise, the use of “or” means “and/or”. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The alkyl group could also be a “lower alkyl” having 1 to 6 carbon atoms.

As used herein, C₁-Cₓ includes C₁-C₂, C₁-C₃ . . . C₁-Cₓ.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl, fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)₁N(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and Re is a straight or branched alkylene or alkenylene chain, and where each of the above substituents is unsubstituted unless otherwise indicated.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

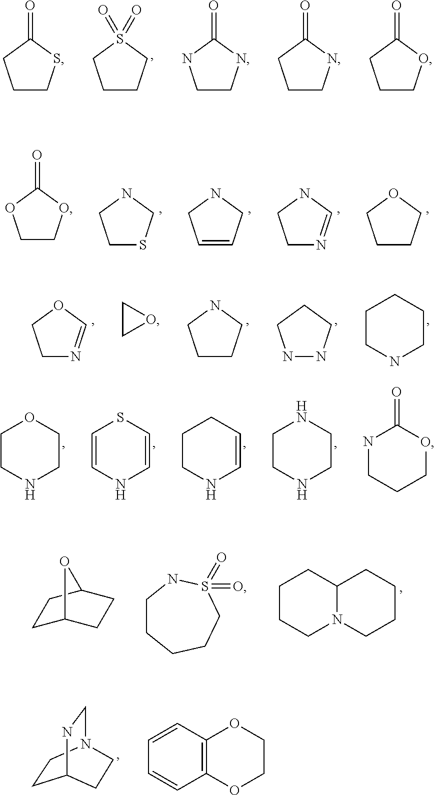

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and Re is a straight or branched alkylene or alkenylene chain, and where each of the above substituents is unsubstituted unless otherwise indicated.

“Sulfonyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

An “alkoxy” group refers to a (alkyl)O— group, where alkyl is as defined herein.

An “aryloxy” group refers to an (aryl)O— group, where aryl is as defined herein.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

“Carboxy” means a —C(O)OH radical.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that may form the protective derivatives of the above substituents are known to those of skill in the art and may be found in references such as Greene and Wuts, above.

The term “target” refers to a biological molecule wherein a protein modulator can interact with. Non-limiting examples of targets include proteins, such as cell cycle regulators, transcription factors, translation initiation factors, cyclins, receptors, cell signaling proteins, ligands, enzymes, and kinases.

The term “drug” as used herein refers to a protein modulator. Non-limiting examples of protein modulators include kinase inhibitors, kinase antagonists, and kinase agonists. For example, a drug can be a BTK inhibitor. In another example, a drug is a BMK antagonist.

The term “agent” refers to a compound that interacts with a target. In some instances, the agent is identical to the drug. In other instances, the agent is similar to the drug. In another instance, the agent is different from the drug.

The term “probe” refers to a compound or molecule for the detection of a target. In some instances, the probe comprises an agent, a linker, a label, or any combination thereof. In some instances, the probe comprises an agent. In other instances, the probe comprises an agent and a linker. In another instance, the probe comprises an agent and a label. In another instance, the probe comprises a label. In some instances, the probe comprises a label and a linker. In some instances, the probe comprises an agent, a linker, and a label. In some instances, the agent is attached to the linker. In other instances, the label is attached to the linker. In some embodiments, the agent is attached to the label via the linker. Alternatively, the agent is attached to the label.

The term “unoccupied target” refers to a target wherein a drug is not bound to.

As used herein, the term “drug-occupied target” or “drug-bound target” refers to a target wherein one or more drugs are bound to. Binding comprises any type of bond, including, but not limited to, covalent, non-covalent, ionic, hydrogen, disulfide, or van der Waals. Binding can also include hydrophilic or hydrophobic interactions.

The term “probe-bound target,” or “probe-bound kinase,” refers to a target, or kinase, wherein one or more probes are bound to. Binding comprises any type of bond, including, but not limited to, covalent, non-covalent, ionic, hydrogen, disulfide, van der Waals. Binding can also include hydrophilic or hydrophobic interactions. In some instances, a “probe-bound target” comprises a drug-occupied target with a probe attached thereto. In other instances, a “probe-bound target” comprises an unoccupied target with a probe attached thereto.

A “treated sample” refers to a sample wherein one or more drugs have been administered to. As used herein, a treated sample from a patient means that the sample is from a patient that has been administered one or more drugs (e.g., a TEC family kinase inhibitor).

An “untreated sample” refers to a sample wherein a drug has not been administered to. As used herein, an untreated sample from a patient means that the sample is from a patient that has not been administered one or more drugs (e.g., a TEC family kinase inhibitor).

### Protein Occupancy Assay

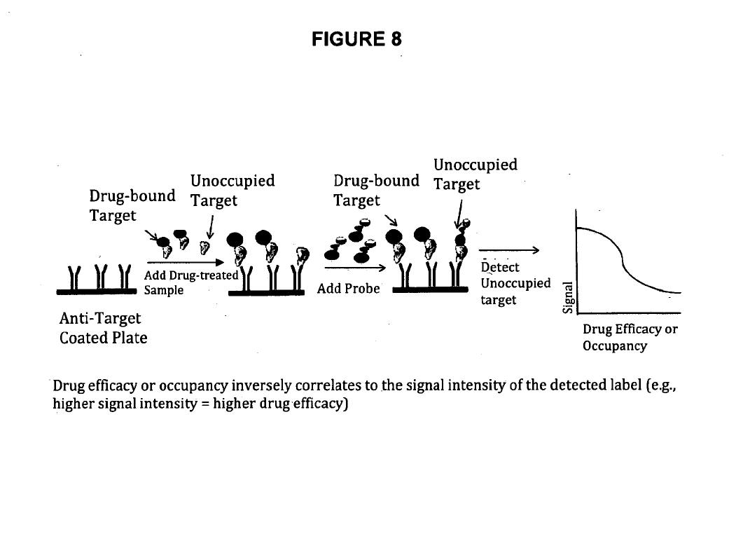

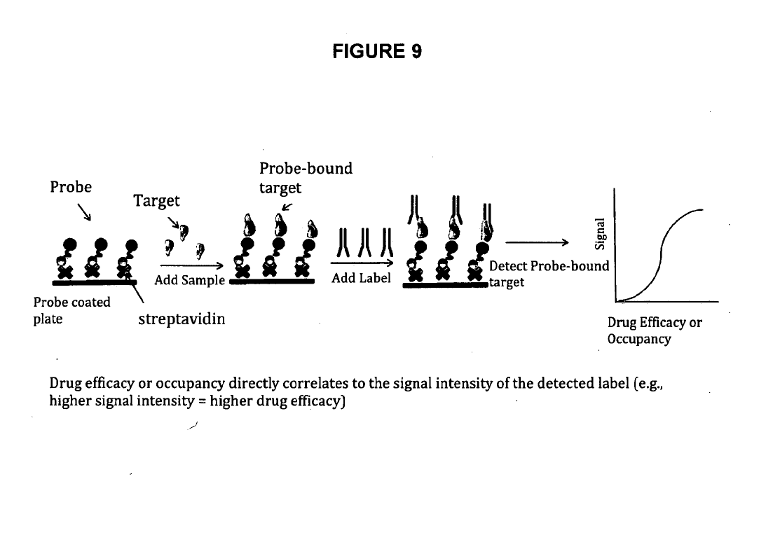

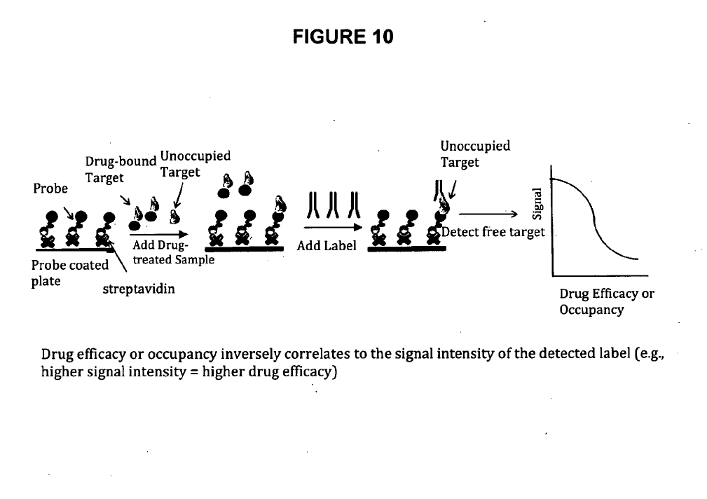

Disclosed herein are methods for determining the efficacy of a protein modulator (e.g., inhibitor drug) on a target (e.g., target protein kinase). In some embodiments, methods are provided for determining the efficacy of a TEC family kinase inhibitor on a target kinase (e.g., a TEC family kinase or homologous kinase). In some embodiments, the method comprises: (a) contacting a sample comprising a TEC family kinase with a probe to form a probe-bound target kinase; (b) detecting the amount of the probe-bound target kinase in the sample; and (c) determining the efficacy of the TEC family kinase inhibitor based on the amount of probe-bound target kinase. In some embodiments, the method further comprises contacting the sample with the TEC family kinase inhibitor prior to step (a) (e.g., combining the sample with the probe). In some embodiments, detecting the amount of the probe-bound target kinase comprises administering a compound, reagent or buffer to detect the probe-bound kinase. In some embodiments, the compound, reagent or buffer comprises horseradish peroxidase (HRP), detection antibody buffer, read buffer, wash buffer. In some embodiments, detecting the presence or absence of the probe-bound target kinase comprises quantifying the amount of probe-bound target kinase. In some embodiments, the quantifying step comprises fluorescence, immunofluorescence, chemiluminescence, or electrochemiluminescence. In some embodiments, determining the efficacy of the TEC family kinase inhibitor comprises determining occupancy of the target kinase by the TEC family kinase inhibitor. In some embodiments, the amount of probe-bound target kinase inversely correlates with the efficacy of the TEC family kinase inhibitor. For example, as shown in FIGS. 8 and 10, if a drug-treated sample (e.g., a sample that is contacted with the drug prior to contact with the probe) is contacted with the probe, then as the amount of probe-bound target kinases (e.g., unoccupied target kinases) detected increases, the efficacy of the drug decreases. In another example, if a drug-treated sample is contacted with the probe, then as the amount of probe-bound target kinase (e.g., unoccupied target kinases) detected decreases, the efficacy of the drug increases. In some embodiments, the amount of probe-bound target kinases directly correlates with the efficacy of the drug. For example, as shown in FIG. 9, if an untreated sample (e.g., a sample that is not contacted with the drug prior to contact with the probe) is contacted with the probe, then as the amount of probe-bound target kinase detected increases, the efficacy of the drug also increases. In another example, if an untreated sample (e.g., a sample that is not contacted with the drug prior to contact with the probe) is contacted with the probe, then as the amount of probe-bound target kinase detected decreases, the efficacy of the drug decreases. In some embodiments, a drug is determined to be effective when the drug binds at least about 50% of the target kinases. Alternatively, a drug is determined to be effective when the drug binds at least about 60% of the target kinases. In some embodiments, a drug is determined to be effective when the drug binds at least about 65%, 70%, 75%, 80%, 85%, 90%, 95%, 97%, or 99% of the targets.

In some embodiments, the assay is performed on a sample obtained from a patient that has been administered a TEC family kinase. In some embodiments, the sample is obtained about 1 hour, 2 hours, 4 hours, 6 hours, 8 hours, 10 hours, 12 hours, 14 hours, 16 hours, 18 hours, 20 hours, 22 hours, 24 hours, 30 hours, 36 hours, 42 hours, 48 hours, 3 days, 4, days, 5 days, 6 days, 1 week, 2 weeks or longer after administration of the TEC family kinase inhibitor.

In some embodiments, the probe comprises an agent and a label. In some instances, the agent is fused to the label. In other instances, the agent is attached to the label. In another instance, the agent is attached to the label by a linker. In some embodiments, the agent and the drug are essentially the same. In some embodiments, the probe comprises a label. In some embodiments, the probe comprises a label and a linker. In some embodiments, the agent and the drug are at least about 20% identical, at least about 30% identical, at least about 40% identical, at least about 50% identical, at least about 60% identical, at least about 70% identical, at least about 80% identical, at least about 90% identical, or at least about 95% identical. In other embodiments, the agent and the drug are different. In some embodiments, the agent and the drug are at least about 5% different, at least about 10% different, at least about 20% different, at least about 30% different, at least about 40% different, at least about 50% different, at least about 60% different, at least about 70% different, at least about 80% different, at least about 90% different, or at least about 95% different.

### Targets

As disclosed herein are methods, assays and systems for determining the efficacy of a the TEC family kinase inhibitor on a target kinase (e.g., TEC family kinase or a homologous tyrosine kinase). In some embodiments, the methods provided herein can be adapted to other target proteins, such as, but not limited to, cell cycle regulators, receptors, ligands, transcriptional regulators, transcription initiation factors, enzymes, cell signaling proteins, and other protein kinases. In particular embodiments, the target kinase is a tyrosine kinase. In particular embodiments, the target kinase is a ser/threonine kinase.

In some embodiments, the target kinase is a member of the TEC family of non-receptor tyrosine kinases. The TEC kinase family comprises TEC, BMX (Bone marrow kinase on the X chromosome; also named Etk), BTK (Bruton's tyrosine kinase), ITK (IL-2-inducible T cell kinase; also known as Emt), and Rlk (Resting lymphocyte kinase; also designated TXK). In some instances, the target kinase is BTK. In other instances, the target kinase is ITK. In other instances, the target kinase is TXK. In other instances, the target kinase is BMX. In other instances, the target kinase is TEC.

In some embodiments, the target kinase is a member of the epidermal growth factor receptor (EGFR). In some embodiments, the target kinase is HER1 (EGFR, ErbB1), HER2/c-neu (ErbB2), HER3 (ErbB3) and HER4 (ErbB4), or JAK3.

In some embodiments, the target kinase is a member of the SRC kinase family. In some embodiments, the target kinase is BLK.

Additional exemplary target kinases for use in the methods and compositions provided include, but are not limited to, Abl, activated Cdc42-associated kinase-1 (ACK1), Akt/PKB, Abl-related gene (Arg), apoptosis signal-regulating kinase (Ask-1), Aurora A, Aurora B, Aurora C, Axl, Calcium/calmodulin dependent kinase-Iδ (CaMKIδ), Calcium/calmodulin dependent kinase IIβ (CaMKIIβ), CaMKIIγ, CaMKIIγ, casein kinases (CK, CK1γ1, CK1γ2, CK1γ3), Cyclin-dependent kinases (Cdk), Cyclin-dependent protein kinase-9/cyclin T1 (CDK9/cyclin T1), Casein kinase-2α2 (CK2α2), Chk, c-kit, cdc-like kinase-2 (CLK2), Cot1, C-terminal c-Src kinase (Csk), Death-associated protein kinase-1 (DAPK1), doublecortin and CAM kinase-like-2 (DCAMKL2), Discoidin domain receptors 1 and 2 (DDR1 and DDR2), Eph receptors, Focal adhesion kinase (FAK), Fer, Fibroblast growth factor receptor (FGFR), Fgr, Fns-like tyrosine kinase (Flt), Fms-like tyrosine kinase-4 (Flt4), Fms/CSF-1 R, Fyn, G protein-coupled receptor kinases (GRKs), G protein-coupled receptor kinase-7 (GRK7), Glycogen synthase kinase (GSK), Hematopoietic cell kinase (Hck), Homeodomain-interacting protein kinase-1 (HIPK1), HIPK2, HIPK3, Insulin-like growth factors (IGF), IKB kinase (IKK), insulin receptor, IL-1 receptor associated kinase (IRAK), stress-activated protein kinase 1 (SAPK), Kinase insert domain-containing receptor (KDR), c-Kit, Lck, LIM kinase (LIMK), Lymphocyte-oriented kinase (LOK), Lyn, MAPK/Erk, MAPK-activated protein kinases (MAPKAP K or MK), MAP kinase/Erk Kinase (MEK), Maternal embryonic leucine zipper kinase (MELK), Met, Mer, Misshapen/NIK-related kinase (MINK), Mitogen activated protein kinase kinase (MKK), Mixed lineage kinase-1 (MLK1), Myotonic dystrophy kinase-related Cdc42-binding kinasea (MRCKa), Mitogen-and-stress-activated protein kinase 1 (MSK1), Mammalian STE20-like kinase (MST), Mammalian STE20-like protein kinase-3 (MST3), target of rapamycin (mTOR, FRAP, RAFT), mTor/FKBP12, NIMA-related protein kinase-3 (NEK3), NEK9, P21-activated kinases (PAK), PAK3, PAR-1 kinase, Platelet-derived Growth Factor Receptors (PDGFR), PI (3,4,5) P3-dependent kinase 1 (PDK1), Phosphorylase kinase (PhK), Phosphatidylinositol (PI) 3-kinase, Polo-like kinase-1 (PLK1), PIM kinases, Protein kinase C, PKD2, cGMP-dependent protein kinase-1α (PKG1α), double-stranded RNA-activated protein kinase (PKR), P38-regulated/activated protein kinase (PRAK), Protein tyrosine kinase-5 (PTK5), Proline rich kinase (Pyk)2, Raf kinases (Raf-1, A-Raf, B-Raf), Ret, Receptor-interacting serine/threonine kinase 2 (RIPK2), Ron, Ros, Rse (Brt, BYK, Dtk, Etk3, Sky, Tif, or sea-related receptor tyrosine kinase), Ribosomal protein S6 kinase-4 (Rsk4), P70 S6 kinase, SAPK, Serum- and glucocorticoid-induced kinase (SGK), c-Src, Syk, TGF-β activated kinase (TAK1), thousand and one amino acid protein kinase-1 (TAO1), Tyrosine kinase with Ig- and EGF-homology domains-2 (Tie2/TEK), Tousled-like kinases (TLKs), Trk, Testis specific serine kinases (TSSK), Unc-51-like kinase-2 (ULK2), ULK3, Vaccinia-related kinase-2, Wee, Yes, ZAP-70, and Zipper interacting protein kinase (ZIPK).

### Drugs

As disclosed herein are methods, assays, and systems for determining the efficacy of a drug on a target. Suitable drugs disclosed herein comprise protein modulators. Protein modulators comprise protein inhibitors, protein antagonists, and protein agonists. In some embodiments, the drug is a protein inhibitor. Examples of protein inhibitors include, but are not limited to, protein kinase inhibitors.

In some embodiments, the drug is a protein kinase inhibitor. In some embodiments, the protein kinase inhibitor is a tyrosine kinase inhibitor. In some embodiments, the tyrosine kinase inhibitor is any one of dasatinib, imatinib, nilotinib, sunitinib, gefitinib, erlotinib.

In some embodiments, the tyrosine kinase inhibitor is a TEC family kinase inhibitor. In some embodiments, the tyrosine kinase inhibitor is a BTK inhibitor. In some embodiments, the BTK inhibitor is a reversible BTK inhibitor. In some embodiments, the reversible BTK inhibitor is LFM-A13 or terreic acid. In some embodiments, the BTK inhibitor is an irreversible BTK inhibitor. Examples of irreversible BTK inhibitors include ibrutinib, AVL-291, AVL-101, AVL-292, or ONO-WG-307. In some embodiments, the irreversible BTK inhibitor is ibrutinib. In some instances, the BTK inhibitor is RN486. In some embodiments, the drug is an ITK inhibitor. In some instances, the ITK inhibitor is CTA056. In some embodiments, the drug is a TEC kinase inhibitor. In some embodiments, the drug is a TXK inhibitor. In some embodiments, the drug is a BMX inhibitor. In some embodiments, the drug is a BLK inhibitor.

In some embodiments, the drug inhibits a kinase. In some embodiments, the drug inhibits a tyrosine kinase. In some embodiments, the drug inhibits a receptor tyrosine kinase. In some embodiments, the drug inhibits a non-receptor tyrosine kinase. In some embodiments, the drug inhibits a serine/threonine kinase.

In some instances, the kinase is a member of the AGC kinase family. In other instances, the kinase is a member of the CaM kinase family. In some embodiments, the kinase is a member of the TK kinase family. Alternatively, the kinase is a member of the CKI kinase family. In some embodiments, the kinase is a member of the CMGC kinase family. In some instances, the kinase is a member of the STE kinase family. In some embodiments, the kinase is a member of the STK kinase family. In some instances, the kinase is a member of the TKL kinase family.

### Protein Occupancy Assay Kit

Disclosed herein are protein occupancy assay kits comprising a linker, a label, an agent, or any combination thereof. In one aspect is a protein occupancy assay kit comprising a linker and a label, wherein the linker is capable of attaching the label to an agent and the agent is a protein modulator. In another aspect is a protein occupancy assay kit comprising an agent, a linker, and a label, wherein the linker is capable of attaching to the agent and the label, thereby attaching the agent to the label. In some embodiments is a protein occupancy assay kit comprising a probe, wherein the probe comprises an agent attached to a label. In some embodiments is a protein occupancy assay kit comprising a probe, wherein the probe comprises an agent attached to a linker.

In some embodiments is a protein occupancy assay kit comprising an agent and a solid support, wherein the agent is attached to the solid support. In another embodiment is a protein occupancy assay kit comprising a label and a solid support, wherein the label is attached to the solid support. In another embodiment is a protein occupancy assay kit comprising a probe and a solid support, wherein the probe comprises an agent, a linker, a label, or any combination thereof. In some embodiments is a protein occupancy assay kit comprising a target (e.g., protein) and a solid support, wherein the target is attached to the solid support.

In some aspects, any of the kits disclosed herein further comprise a label. In some aspects, any of the kits disclosed herein further comprise a linker. In some aspects, any of the kits disclosed herein further comprise an agent. In some aspects, any of the kits disclosed herein further comprise a plurality of linkers, wherein the linkers are capable of attaching to another linker, an agent, a label, or any combination thereof. In some aspects, any of the kits disclosed herein further comprise a probe. In some aspects the probe comprises an agent, a linker, a label, or any combination thereof. In some aspects, any of the kits disclosed herein further comprise a target (e.g., protein). Exemplary embodiments of agents, linkers, labels, probes, solid supports, and targets are disclosed herein. Further disclosed herein are exemplary methods for attaching probes or targets to solid supports.

### Probe

In some embodiments, the methods, kits, and compositions disclosed herein comprise a probe. In some embodiments, the probe comprises an agent and a label. In some embodiments, the agent and label are attached. In other embodiments, the probe comprises an agent and a linker. In some embodiments, the agent and linker are attached. In another embodiment, the probe comprises an agent, a linker, and a label. In some embodiments, the agent, linker and/or label are attached to each other. In some embodiments, the probe comprises a label. In another embodiment, the probe comprises a label and a linker. In some embodiments, the label and the linker are attached. In some embodiments, attachment is by chemical methods, enzymatic methods, or crosslinking methods. In some embodiments, the probe is attached to a solid support. Exemplary embodiments of agents, linkers, labels, and solid supports are disclosed herein. Agent

In some embodiments, the methods, kits, and compositions disclosed herein comprise an agent. Suitable agents comprise protein modulators (e g, inhibitors, antagonists, and agonists). In some embodiments, the agent is a drug. Suitable drugs disclosed herein comprise protein modulators. Protein modulators comprise protein inhibitors, protein antagonists, and protein agonists. In some embodiments, the drug is a protein inhibitor. Examples of protein inhibitors include, but are not limited to, protein kinase inhibitors.

In some embodiments, the drug is a protein kinase inhibitor. In some embodiments, the protein kinase inhibitor is a tyrosine kinase inhibitor. In some embodiments, the tyrosine kinase inhibitor is any one of dasatinib, imatinib, nilotinib, sunitinib, gefitinib, erlotinib.

In some embodiments, the tyrosine kinase inhibitor is a TEC family kinase inhibitor. In some embodiments, the tyrosine kinase inhibitor is a BTK inhibitor. In some embodiments, the BTK inhibitor is a reversible BTK inhibitor. In some embodiments, the reversible BTK inhibitor is LFM-A13 or terreic acid. In some embodiments, the BTK inhibitor is an irreversible BTK inhibitor. Examples of irreversible BTK inhibitors include ibrutinib, AVL-291, AVL-101, AVL-292, or ONO-WG-307. In some embodiments, the irreversible BTK inhibitor is ibrutinib. In some instances, the BTK inhibitor is RN486. In some embodiments, the drug is an ITK inhibitor. In some instances, the ITK inhibitor is CTA056. In some embodiments, the drug is a TEC kinase inhibitor. In some embodiments, the drug is a TXK inhibitor. In some embodiments, the drug is a BMX inhibitor. In some embodiments, the drug is a BLK inhibitor.

In some embodiments, the drug inhibits a kinase. In some embodiments, the drug inhibits a tyrosine kinase. In some embodiments, the drug inhibits a receptor tyrosine kinase. In some embodiments, the drug inhibits a non-receptor tyrosine kinase. In some embodiments, the drug inhibits a serine/threonine kinase.

In some instances, the kinase is a member of the AGC kinase family. In other instances, the kinase is a member of the CaM kinase family. In some embodiments, the kinase is a member of the TK kinase family. Alternatively, the kinase is a member of the CKI kinase family. In some embodiments, the kinase is a member of the CMGC kinase family. In some instances, the kinase is a member of the STE kinase family. In some embodiments, the kinase is a member of the STK kinase family. In some instances, the kinase is a member of the TKL kinase family.

### Linker

In some embodiments, the methods, kits, and compositions disclosed herein comprise a linker. Suitable linkers comprise any chemical or biological compound capable of attaching to a label and/or agent disclosed herein. If the linker attaches to both the label and the agent, then a suitable linker would be capable of sufficiently separating the label and the agent. Suitable linkers would not significantly interfere with the ability of the agent to bind to a target (e.g., protein). Suitable linkers would not significantly interfere with the ability of the label to be detected. In some embodiments, the linker is rigid. In other embodiments, the linker is flexible. In another embodiment, the linker is semi rigid. In some embodiments, the linker is proteolytically stable (e.g., resistant to proteolytic cleavage). In another embodiment, the linker is proteolytically unstable (e.g., sensitive to proteolytic cleavage). In some embodiments, the linker is helical. In some embodiments, the linker is non-helical. In some embodiments, the linker is coiled. In some embodiments, the linker is β-stranded. In some embodiments, the linker comprises a turn conformation. In some embodiments, the linker is a single chain. In some embodiments, the linker is a long chain. In some embodiments, the linker is a short chain. In some embodiments, the linker comprises at least about 5 residues, at least about 10 residues, at least about 15 residues, at least about 20 residues, at least about 25 residues, at least about 30 residues, or at least about 40 residues.

Examples of linkers include, but are not limited to, hydrazone, disulfide, thioether, and peptide linkers. In some embodiments, the linker is a peptide linker. In some embodiments, the peptide linker comprises a proline residue. In some embodiments, the peptide linker comprises an arginine, phenylalanine, threonine, glutamine, glutamate, or any combination thereof. In some embodiments, the linker is a heterobifunctional crosslinker. In some embodiments, the heterobifunctional crosslinker is Sulfo-SMCC.

### Label

In some embodiments, the methods, kits, and compositions disclosed herein comprise a label. Examples of labels include, but are not limited to, chemical, biochemical, biological, colorimetric, enzymatic, fluorescent, luminescent labels, chemiluminescent labels, and electrochemiluminescent labels, which are well known in the art. In some embodiments, the label is selected from the group consisting of a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof.

In some embodiments, the label is a chemical label. Examples of chemical labels can include, but are not limited to, biotin and radioisotopes (e.g., iodine, carbon, phosphate, hydrogen).

In some embodiments, the methods, kits, and compositions disclosed herein comprise a biological label. In some embodiments, biological labels comprise metabolic labels, including, but not limited to, bioorthogonal azide-modified amino acids, sugars, and other compounds.

In some embodiments, the methods, kits, and compositions disclosed herein comprise an enzymatic label. Enzymatic labels can include, but are not limited to horseradish peroxidase (HRP), alkaline phosphatase (AP), glucose oxidase, and β-galactosidase. In some embodiments, the enzymatic label is luciferase.

In some embodiments, the methods, kits, and compositions disclosed herein comprise a fluorescent label. In some embodiments, the fluorescent label is an organic dye (e.g., FITC), biological fluorophore (e.g., green fluorescent protein), or quantum dot. A non-limiting list of fluorescent labels includes fluorescein isothiocyante (FITC), DyLight Fluors, fluorescein, rhodamine (tetramethyl rhodamine isothiocyanate, TRITC), coumarin, Lucifer Yellow, and BODIPY. In some embodiments, the label is a fluorophore. Exemplary fluorophores include, but are not limited to, indocarbocyanine (C3), indodicarbocyanine (C5), Cy3, Cy3.5, Cy5, Cy5.5, Cy7, Texas Red, Pacific Blue, Oregon Green 488, Alexa Fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX™), LIZ™, VIC™, NED™, PET™, SYBR, PicoGreen, RiboGreen, and the like. In some embodiments, the fluorescent label is a green fluorescent protein (GFP), red fluorescent protein (RFP), yellow fluorescent protein, phycobiliproteins (e.g., allophycocyanin, phycocyanin, phycoerythrin, and phycoerythrocyanin).

### Solid Support

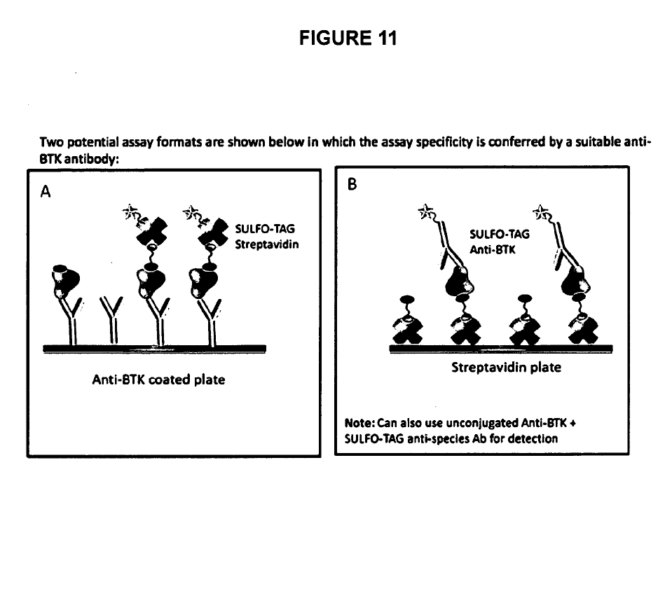

In some embodiments, the methods, kits, and compositions disclosed herein comprise a solid support. A solid support comprises any solid platform to which a probe or antibody can be attached. In some embodiments, the solid support comprises a bead, plate, and array. In some embodiments, the solid support comprises a bead attached to a plate. For example, as shown in FIG. 11B, a streptavidin bead is attached to a plate. In some embodiments, the solid support comprises a plate. In another embodiment, the solid support comprises an antibody attached to a plate. For example, as shown in FIG. 11A, an anti-BTK antibody is attached to a plate.

In some embodiments, the methods, kits, and compositions disclosed herein comprise a bead. Examples of beads include, but are not limited to, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

In some embodiments, the methods, kits, and compositions disclosed herein comprise a plate. Examples of plates include, but are not limited to, MSD multi-array plates, MSD Multi-Spot® plates, microplate, ProteOn microplate, AlphaPlate, DELFIA plate, IsoPlate, and LumaPlate.

Methods for Attaching Agents, Linkers, and/or Labels

In some embodiments, the methods, kits, and compositions disclosed herein comprise an agent, linker, label, or any combination thereof. In some embodiments, the agent, linker, and/or label are attached. Methods for attaching agents, linkers, and/or labels include, but are not limited to, chemical labeling and enzymatic labeling.

In some embodiments, methods to attach labels to linkers and/or agents comprise chemical labeling techniques. In some embodiments, chemical labeling techniques comprise a chemically reactive group. Common reactive groups include, but are not limited to, amine-reactive isothiocyanate derivatives including FITC, amine-reactive succinimidyl esters such as NHS-fluorescein or NHS-rhodamine, and sulfhydryl-reactive maleimide-activated fluors such as fluorescein-5-maleimide. In some embodiments, reaction of any of these reactive dyes with another molecule results in a stable covalent bond formed between a fluorophore and the linker and/or agent. In some embodiments, the reactive group is isothiocyanates. In some embodiments, a label is attached to an agent through the primary amines of lysine side chains. In some embodiments, chemical labeling comprises a NHS-ester chemistry method.

In some embodiments, methods to attach labels to linkers and/or agents comprise enzymatic labeling and affinity labeling. Enzymatic labeling can include, but is not limited to, an acyl carrier protein/phosphopantetheine transferase (ACP/PPTase), Q-tag/transglutaminase (TGase) (Lin, C. W. and Ting, A. Y. Transglutaminase-catalyzed site-specific conjugation of small-molecule probes to proteins in vitro and on the surface of living cells. J. Am. Chem. Soc. 2006, 128, 4542-4543), biotin acceptor peptide/biotin ligase (AP/Bir A) (Chen, I., et al., Site-specific labeling of cell surface proteins with biophysical probes using biotin ligase. Nat. Meth. 2005, 2, 99-104.), farnesylation motif/protein farnesyltransferase (PFTase) (Duckworth, B. P., et al., Selective labeling of proteins by using protein farnesyltransferase. ChemBioChem 2007, 8, 98-105), aldehyde tag/formylglycine-generating enzyme (Carrico, I. S., et al., Introducing genetically encoded aldehydes into proteins. Nat. Chem. Biol. 2007, 3, 321-322), human O⁶-alkylguanine transferase (hAGT) (Keppler, A., et al., A general method for the covalent labeling of fusion proteins with small molecules in vivo. Nat. Biotechnol. 2003, 21, 86-89; Keppler, A., et al., Labeling of fusion proteins with synthetic fluorophores in live cells. Proc. Natl. Acad. Sci. USA 2004,101, 9955-9959), and mutated prokaryotic dehalogenase (HaloTag™) methods (Los, G., et al., Halotag™ technology: cell imaging and protein analysis. Cell Notes 2006, 14, 10-14). Affinity labeling can include, but is not limited to, noncovalent methods utilizing dihydrofolate reductase (DHFR) (Miller, L. W., et al., Methotrexate conjugates: a molecular in vivo protein tag. Angew. Chem. Int. Ed. Engl. 2004, 43, 1672-1675; Miller, L. W., et al., In vivo protein labeling with trimethoprim conjugates: a flexible chemical tag. Nat. Meth. 2005, 2, 255-257) and Phe36Val mutant of FK506-binding protein 12 (FKBP12(F36V)) (Marks, K. M., Braun, P. D., Nolan, G. P., A general approach for chemical labeling and rapid, spatially controlled protein inactivation. Proc. Natl. Acad. Sci. USA 2004, 101, 9982-9987), as well as metal-chelation methods.

In some embodiments, crosslinking reagents are used to attach labels, linkers, and/or agents. In some embodiments, the crosslinking reagent is glutaraldehyde. In some embodiments, glutaraldehyde reacts with amine groups to create crosslinks by one of several routes. In some embodiments, under reducing conditions, the aldehydes on both ends of glutaraldehyde couple with amines to form secondary amine linkages.

In some embodiments, attachment of labels, linkers, and/or agents comprises periodate-activation followed by reductive amination. For example, preiodate-activation followed by reductive amination is used to conjugate of HRP and other glycoproteins to a linker and/or agent. In some instances, treatment of a glycosylated enzyme with periodate results in oxidation of sugar cis-diol groups (especially sialic acid, which is common in glycoprotein polysaccharides), resulting in formation of aldehyde groups. In some instances, these aldehyde groups react (in the presence of the mild reductant cyanoborohydride) with primary amines of an added antibody or other molecule.

In some embodiments, Sulfo-SMCC or other heterobifunctional crosslinkers are used to conjugate labels to linkers and/or agents. For example, Sulfo-SMCC is used to conjugate an enzyme to a drug. In some embodiments, the enzyme is activated and purified in one step and then conjugated to the drug in a second step. In some embodiments, the directionality of crosslinking is limited to one specific orientation (e.g., amines on the enzyme to sulfhydryl groups on the antibody).

In some embodiments, a linkage is formed between the linker and the label and/or agent. The term “linkage,” as used herein to refer to bonds or a chemical moiety formed from a chemical reaction between the functional group of a linker and another molecule (e.g., label, agent). In some embodiments, such bonds include, but are not limited to, covalent linkages and non-covalent bonds, while such chemical moieties include, but are not limited to, esters, carbonates, imines, phosphate esters, hydrazones, acetals, orthoesters, peptide linkages, and oligonucleotide linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages means that the linkages are degradable in water or in aqueous solutions, including for example, blood. In other embodiments, enzymatically unstable or degradable linkages means that the linkage is degraded by one or more enzymes. By way of example only, PEG and related polymers include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

### Methods for Attaching Probes or Targets (e.g., Proteins) to Solid Supports

In some embodiments, methods for attaching probes or targets (e.g., proteins) to solid supports include chemical and/or enzymatic methods. In some embodiments, the chemical methods are disclosed herein. In some embodiments, the enzymatic methods disclosed herein. In some embodiments, methods for attaching probes or targets to a solid support comprises coating the solid support with a probe or target. Methods for coating a microplate with an antibody are well known in the art and can include diluting the antibody in a coating buffer and adding the diluted antibody to a well in the microplate. The unbound antibody can be removed by washing the plate with a wash buffer.

### TEC Family Kinase Probe Compounds

The TEC family kinase probe compounds described herein are composed of a moiety comprising a TEC family kinase inhibitor, a linker moiety, and a detectable label. In some embodiments, the TEC family kinase inhibitor is an irreversible TEC family kinase inhibitor. In some embodiments, the TEC family kinase inhibitor is a Btk inhibitor. In some embodiments, the inhibitor of Btk is an irreversible inhibitor. In another embodiment, the irreversible inhibitor of Btk binds to a non-catalytic residue in the ATP binding pocket of Btk. In further embodiments, the non-catalytic residue is a cysteine residue. In some embodiments, the Btk probe forms a covalent bond with at least one non-catalytic residue of Btk. In some embodiments, the TEC family kinase probe compound is a derivative of an irreversible Btk inhibitor. In some embodiments, the TEC family kinase probe compound is a derivative of ibrutinib. In some embodiments, the TEC family kinase probe compound is a derivative of ibrutinib. In some embodiments, the TEC family kinase probe compound consists of ibrutinib attached to a label via a linker. In some embodiments, the TEC family kinase probe compound is a derivative of AVL-292, AVL-291, AVL-101, CNX-774, or ONO-WG-307. In some embodiments, the TEC family kinase probe compound consists of AVL-292, AVL-291, AVL-101, CNX-774, or ONO-WG-307 attached to a label via a linker.

In one aspect is a TEC family kinase probe of Formula (I) comprising:

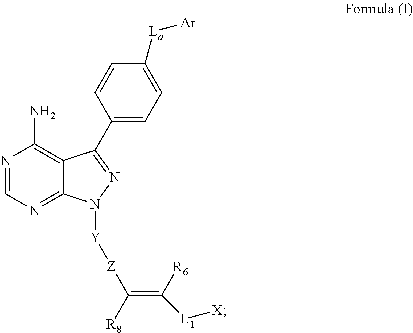

wherein:
Lₐ is CH₂, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), S(O)ₙ, OS(O)ₙ, NHS(O)ₙ, where n is 1 or 2;
R₆ and R₈ are independently selected from H, optionally substituted alkyl, or optionally substituted heteroalkyl;
L₁ is selected from the group consisting of a bond, optionally substituted alkyl, optionally substituted heteroalkyl, optionally substituted cycloalkyl, optionally substituted heterocycloalkyl, optionally substituted aryl, optionally substituted heteroaryl, an optionally substituted amide moiety, an optionally ketone moiety, an optionally substituted carbamate moiety, and an optionally ester moiety, or any combination thereof; and
X is a detectable label.

In some embodiments, Lₐ is CH₂, O, or NH. In other embodiments, Lₐ is O or NH. In some embodiments, Lₐ is O. In some embodiments, Ar is a substituted or unsubstituted aryl. In some embodiments, Ar is a 6-membered aryl. In some other embodiments, Ar is phenyl. In some embodiments, Z is C(═O), OC(═O), NHC(═O), S(═O)ₙ, OS(═O)₂, or NHS(═O)₂. In some embodiments, Z is C(═O), NHC(═O), or S(═O)₂. In some embodiments, Z is C(═O). In some embodiments, Z is NHC(═O). In some embodiments, Y is an optionally substituted group selected from among alkyl, heteroalkyl, cycloalkyl, and heterocycloalkyl. In some embodiments, Y is an optionally substituted group selected from among C₁-C₆alkyl, C₁-C₆heteroalkyl, 4-, 5-, 6- or 7-membered cycloalkyl, and 4-, 5-, 6- or 7-membered heterocycloalkyl. In some embodiments, Y is an optionally substituted group selected from among C₁-C₆alkyl, C₁-C₆heteroalkyl, 5-, or 6-membered cycloalkyl, and 5-, or 6-membered heterocycloalkyl containing 1 or 2 N atoms. In some embodiments, Y is a 5-, or 6-membered cycloalkyl, or a 5-, or 6-membered heterocycloalkyl containing 1 or 2 N atoms. In some embodiments, Y is a pyrrolidine ring. In some embodiments, Y is a piperidine ring. In some embodiments, R₆ and R₈ are independently selected from among H, unsubstituted C₁-C₄ alkyl, substituted C₁-C₄alkyl, unsubstituted C₁-C₄heteroalkyl, and substituted C₁-C₄heteroalkyl. In some embodiments, R₆ and R₈ are each H.

In some embodiments, L₁ is selected from the group consisting of a bond, optionally substituted alkyl, optionally substituted heteroalkyl, optionally substituted cycloalkyl, optionally substituted heterocycloalkyl, optionally substituted aryl, optionally substituted heteroaryl, an optionally substituted amide moiety, an optionally ketone moiety, an optionally substituted carbamate moiety, and an optionally ester moiety. In some embodiments, L₁ is selected from any combination of at least two groups selected from optionally substituted alkyl, optionally substituted heteroalkyl, optionally substituted cycloalkyl, optionally substituted heterocycloalkyl, optionally substituted aryl, optionally substituted heteroaryl, an optionally substituted amide moiety, an optionally ketone moiety, an optionally substituted carbamate moiety, and an optionally ester moiety. In some embodiments, L₁ is selected from any combination of at least three groups selected from optionally substituted alkyl, optionally substituted heteroalkyl, optionally substituted cycloalkyl, optionally substituted heterocycloalkyl, optionally substituted aryl, optionally substituted heteroaryl, an optionally substituted amide moiety, an optionally ketone moiety, an optionally substituted carbamate moiety, and an optionally ester moiety. In some embodiments, L₁ is selected from any combination of at least four groups selected from optionally substituted alkyl, optionally substituted heteroalkyl, optionally substituted cycloalkyl, optionally substituted heterocycloalkyl, optionally substituted aryl, optionally substituted heteroaryl, an optionally substituted amide moiety, an optionally ketone moiety, an optionally substituted carbamate moiety, and an optionally ester moiety.

In some embodiments, X is a detectable label selected from the group consisting of a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, or a combination thereof. In some embodiments, X is a fluorophore. In some embodiments, X is biotin. In some embodiments, X is biotin analogue.

In another embodiment is a TEC family kinase probe of Formula (II) comprising:

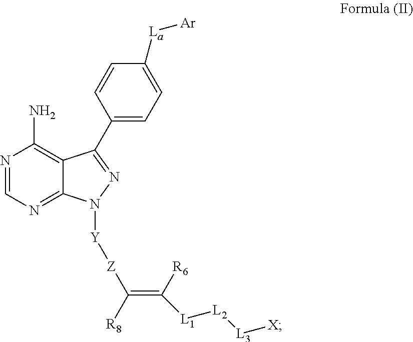

wherein:
Lₐ is CH₂, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), OS(O)ₙ, NHS(O)ₙ, where n is 1 or 2;
R₆ and R₈ are independently selected from H, optionally substituted alkyl, or optionally substituted heteroalkyl;
L₁ is optionally substituted alkyl or optionally substituted heteroalkyl;
L₂ is a bond, optionally substituted heterocycloalkyl, or —N(H)C(O)(CH₂)ₘC(O)N(H)—, where m is 2-6;
L₃ is optionally substituted alkyl or optionally substituted heteroalkyl; and
X is a detectable label.

In some embodiments, Lₐ is CH₂, O, or NH. In other embodiments, Lₐ is O or NH. In some embodiments, Lₐ is O. In some embodiments, Ar is a substituted or unsubstituted aryl. In some embodiments, Ar is a 6-membered aryl. In some other embodiments, Ar is phenyl. In some embodiments, Z is C(═O), OC(═O), NHC(═O), S(═O)ₙ, OS(═O)₂, or NHS(═O)₂. In some embodiments, Z is C(═O), NHC(═O), or S(═O)₂. In some embodiments, Z is C(═O). In some embodiments, Z is NHC(═O). In some embodiments, Y is an optionally substituted group selected from among alkyl, heteroalkyl, cycloalkyl, and heterocycloalkyl. In some embodiments, Y is an optionally substituted group selected from among C₁-C₆alkyl, C₁-C₆heteroalkyl, 4-, 5-, 6- or 7-membered cycloalkyl, and 4-, 5-, 6- or 7-membered heterocycloalkyl. In some embodiments, Y is an optionally substituted group selected from among C₁-C₆alkyl, C₁-C₆heteroalkyl, 5-, or 6-membered cycloalkyl, and 5-, or 6-membered heterocycloalkyl containing 1 or 2 N atoms. In some embodiments, Y is a 5-, or 6-membered cycloalkyl, or a 5-, or 6-membered heterocycloalkyl containing 1 or 2 N atoms. In some embodiments, Y is a pyrrolidine ring. In some embodiments, Y is a piperidine ring. In some embodiments, R₆ and R₈ are independently selected from among H, unsubstituted C₁-C₄ alkyl, substituted C₁-C₄alkyl, unsubstituted C₁-C₄heteroalkyl, and substituted C₁-C₄heteroalkyl. In some embodiments, R₆ and R₈ are each H.

In some embodiments, L₁ is optionally substituted alkyl. In some embodiments, L₁ is optionally substituted heteroalkyl. In some embodiments, L₂ is a bond. In some embodiments, L₂ is optionally substituted heterocycloalkyl. In some embodiments, L₂ is optionally substituted piperazine. In some embodiments, L₂ is optionally substituted piperidine. In some embodiments, L₂ is —N(H)C(O)(CH₂)₂C(O)N(H)—. In some embodiments, L₂ is —N(H)C(O)(CH₂)₃C(O)N(H)—. In some embodiments, L₂ is —N(H)C(O)(CH₂)₄C(O)N(H)—. In some embodiments, L₂ is —N(H)C(O)(CH₂)₅C(O)N(H)—. In some embodiments, L₂ is —N(H)C(O)(CH₂)₆C(O)N(H)—. In some embodiments, L₃ is optionally substituted alkyl. In some embodiments, L₃ is optionally substituted heteroalkyl.

In some embodiments, X is a detectable label selected from the group consisting of a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, or a combination thereof. In some embodiments, X is a fluorophore. In some embodiments, X is biotin. In some embodiments, X is biotin analogue.

In another embodiment is a TEC family kinase probe of Formula (III) comprising:

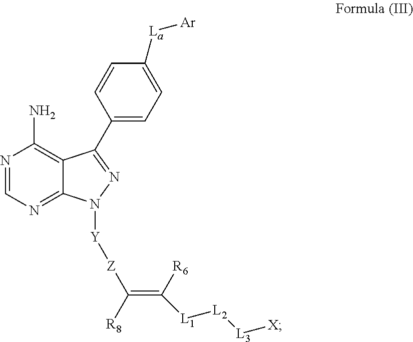

wherein:
Lₐ is O;
Ar is optionally substituted phenyl;
Y is optionally substituted cycloalkyl or optionally substituted heterocycloalkyl;
Z is C(O) or NHC(O);
R₆ and R₈ are independently selected from H, optionally substituted alkyl, or optionally substituted heteroalkyl;
L₁ is optionally substituted alkyl or optionally substituted heteroalkyl;
L₂ is a bond, optionally substituted heterocycloalkyl, or —N(H)C(O)(CH₂)ₘC(O)N(H)—, where m is 2-6;
L₃ is optionally substituted alkyl or optionally substituted heteroalkyl; and
X is

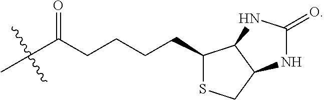 [SMILES: *C(=O)CCCC[C@@H]1SC[C@@H]2NC(=O)N[C@@H]21]

In some embodiments, Z is C(═O). In some embodiments, Z is NHC(═O). In some embodiments, Y is an optionally substituted cycloalkyl. In some embodiments, Y is an optionally substituted heterocycloalkyl. In some embodiments, Y is a 5-, or 6-membered cycloalkyl, or a 5-, or 6-membered heterocycloalkyl containing 1 or 2 N atoms. In some embodiments, Y is a cyclohexyl ring. In some embodiments, Y is a pyrrolidine ring. In some embodiments, Y is a piperidine ring. In some embodiments, R₆ and R₈ are independently selected from among H, unsubstituted C₁-C₄ alkyl, substituted C₁-C₄alkyl, unsubstituted C₁-C₄heteroalkyl, and substituted C₁-C₄heteroalkyl. In some embodiments, R₆ and R₈ are each H.

In some embodiments, L₁ is optionally substituted alkyl. In some embodiments, L₁ is optionally substituted heteroalkyl. In some embodiments, L₂ is a bond. In some embodiments, L₂ is optionally substituted heterocycloalkyl. In some embodiments, L₂ is optionally substituted piperazine. In some embodiments, L₂ is optionally substituted piperidine. In some embodiments, L₂ is —N(H)C(O)(CH₂)₂C(O)N(H)—. In some embodiments, L₂ is —N(H)C(O)(CH₂)₃C(O)N(H)—. In some embodiments, L₂ is —N(H)C(O)(CH₂)₄C(O)N(H)—. In some embodiments, L₂ is —N(H)C(O)(CH₂)₅C(O)N(H)—. In some embodiments, L₂ is —N(H)C(O)(CH₂)₆C(O)N(H)—. In some embodiments, L₃ is optionally substituted alkyl. In some embodiments, L₃ is optionally substituted heteroalkyl.

In some embodiments, the probe comprises biotin attached to ibrutinib via a linker (i.e. a biotinylated ibrutinib). In some embodiments, the probe is selected from among:

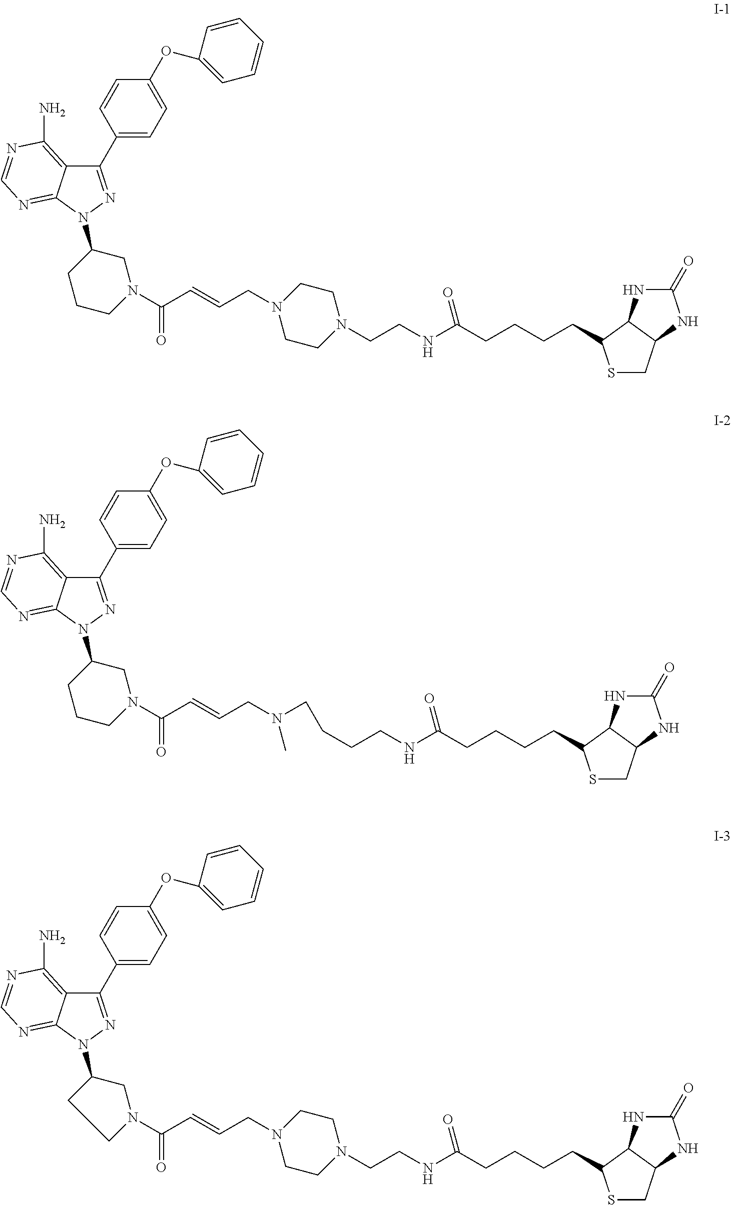

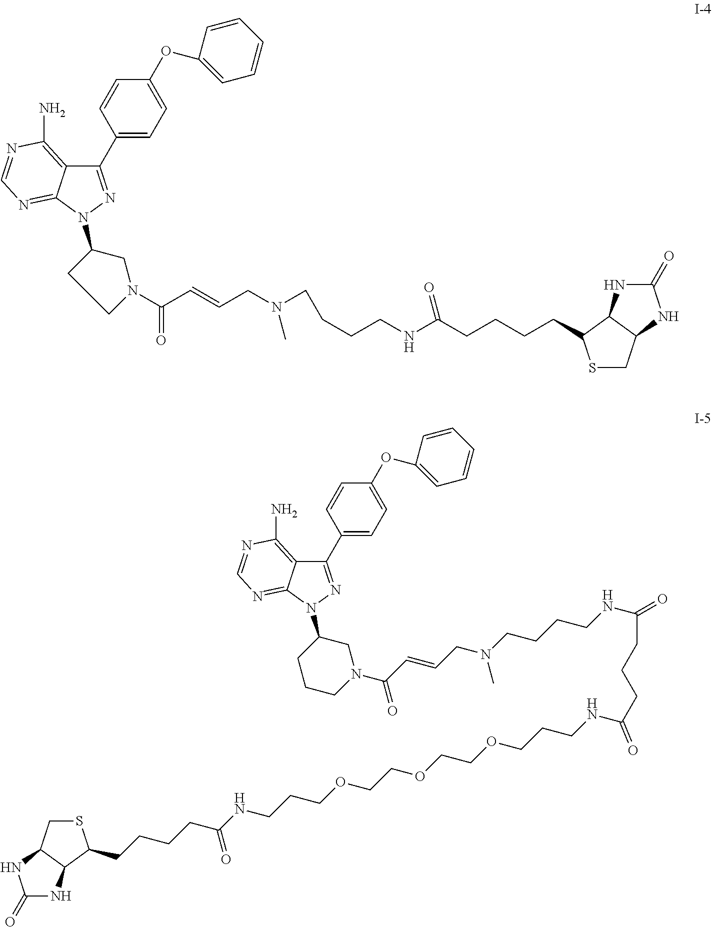

### Detection Methods

In some embodiments, the methods, assays, and systems disclosed herein comprise detection of the targets (e.g., the target kinases). In some instances, the targets are probe-bound targets. In some instances, the probe-bound targets are drug-occupied targets. In other instances, the probe-bound targets are unoccupied targets.

In some embodiments, detection of the targets comprises contacting the sample with an antibody. In some embodiments, the antibody is labeled antibody. In some embodiments, the antibody is labeled with an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the labeled antibody is a SULFO TAG labeled antibody. In some embodiments, the labeled antibody is a horseradish peroxidase labeled antibody. In some embodiments, the antibody is used as a primary antibody. In another embodiment, the antibody is used as a secondary antibody.

In some embodiments, detection of the targets comprises chemiluminescence, luminescence, fluorescence, immunofluorescence, calorimetry, or electrochemiluminescence methods. In some embodiments, detection of the targets comprises a fluorescence detection instrument. In some embodiments, the fluorescence detection instrument comprises an excitation light source. In some embodiments, the light source is a laser, photodiode, or lamps. In some embodiments, the lamp is a xenon arc or mercury. In some embodiments, the fluorescence detection instrument comprises a fluorophore. In some embodiments, the fluorescence detection instrument comprises a filter. In some embodiments, the filter isolates specific wavelengths to excite different fluorophores. In some embodiments, the fluorescence detection instrument comprises a detector that records the output. In some embodiments, the output is an electronic signal. In some embodiments, the fluorescence detection instrument is a fluorescent microscope. In some embodiments, the fluorescent microscope detects the localized fluors. In some embodiments, detection occurs in two and/or three dimensions. In some embodiments, the fluorescence detection instrument is a fluorescence scanner. In some embodiments, the fluorescence scanner is a microarray reader. In some embodiments, the microarray reader detects localized fluors in two dimensions. In some embodiments, the fluorescence detection instrument is a spectrofluorometer. In some embodiments, the fluorescence detection instrument is a microplate reader. In some embodiments, the fluorescence detection instrument records the average fluorescence. In some embodiments, the fluorescence detection instrument is a flow cytometer. In some embodiments, the flow cytometer analyzes the fluorescence of individual cells in a sample population.

In some embodiments, detection of the targets comprises the use of a microplate reader. In some instances, the microplate reader is an xMark™ microplate absorbance spectrophotometer, iMark microplate absorbance reader, EnSpire® Multimode plate reader, EnVision Multilabel plate reader, VICTOR X Multilabel plate reader. Fluoroskan Ascent FL Microplate Fluoremeter and Luminometer, Fluoroskan Ascent Microplate Fluoremeter, Luminoskan Ascent Microplate Luminometer, Multiskan EX Microplate Photometer, Muliskan FC Microplate Photometer, and Muliskan GO Microplate Photometer. In some instances, the microplate reader detects absorbance, fluorescence, luminescence, time-resolved fluorescence, and light scattering. In some embodiments, the microplate reader detects dynamic light scattering. Alternatively, the microplate reader detects static light scattering.

In some embodiments, detection of the targets comprises the use of a microplate imager. In some instances, the microplate imager comprises ViewLux uHTS microplate imager and BioRad microplate imaging system.

In some embodiments, computer-based systems are employed in the detection methods for determining protein occupancy described herein. In some embodiments, the computer-based systems include a digital processing device which analyzes the data and signals obtained from an devices or instrument such as a multiwell plate assay. In some embodiments, provided herein is computer readable storage media encoded with a computer program including instructions executable by a digital processing device for performance of the detection methods for determining protein occupancy described herein.

In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In some embodiments, provided herein is an analytical system for determining protein occupancy of a target kinase, comprising: (a) a probe ELISA assay comprising patient samples comprising a target kinase and a probe as described herein; (b) an analytical instrument for detecting probe-bound target kinases for determining protein occupancy; (c) a digital processing device comprising an operating system configured to perform executable instructions and a memory; and (d) a computer program, provided to the digital processing device, including executable instructions that create a target occupancy application comprising: (i) a database of threshold levels for target occupancy; (ii) a software module configured to receive signal data from the analytical instrument; (iii) a software module configured to apply an algorithm to the signal data to identify the level of target kinase occupancy in the sample.

### Applications

### Drug Research and Validation

Any of the assays and systems disclosed herein can be useful in researching and validating drugs. Provided herein are methods for validating a drug comprising (a) contacting a sample comprising a target with a probe to form a probe-bound target; (b) detecting the presence or absence of the probe-bound target; and (c) determining occupancy of the target by a drug based on the presence or absence of the probe-bound target, thereby validating the drug.

Further provided herein are methods for determining occupancy of a target comprising: a) combining a sample comprising a target with a probe; b) detecting the presence or absence of a probe-bound target; and c) determining occupancy of the target by a drug based on the presence or absence of the probe-bound target.

In some embodiments, the method further comprises capturing the target prior to step (a) contacting the sample with the probe. In some embodiments, the target is captured by an antibody. In some embodiments, the antibody is an anti-target antibody. In some embodiments, the antibody is attached to a solid support. In some embodiments, the solid support is a microplate. In some embodiments, the microplate is a MSD microplate.

In some embodiments, the method further comprises contacting the probe-bound target with a primary detection agent. In some embodiments, the primary detection agent comprises an antibody, a bead, a dye, or a fluorophore. In some embodiments, the primary detection agent comprises an antibody. In some embodiments, the antibody is an anti-BTK antibody. In some embodiments, the method further comprises contacting the detection agent with a secondary detection agent. In some embodiments, the secondary detection agent comprises an antibody, a bead, a dye, or a fluorophore. In some embodiments, the primary detection agent is labeled. In some embodiments, the secondary detection agent is labeled. In some embodiments, the label is an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the label is a SULFO TAG.

In some embodiments, detecting the presence or absence of the probe-bound target comprises contacting the sample with a solid support. In some embodiments, the solid support comprises a bead. In some embodiments, the bead is a streptavidin bead. In some embodiments, the bead is a magnetic bead. In some embodiments, the bead is a labeled bead. In some embodiments, the bead is a labeled streptavidin bead. In some embodiments, the bead is a labeled with an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, the bead is a SULFO TAG bead. In some embodiments, the bead is a SULFO TAG streptavidin bead.

In some embodiments, the bead interacts with the probe. In some embodiments, the probe comprises a label. In some embodiments, the label comprises biotin. In some embodiments, the bead interacts with biotin. In some embodiments, the bead forms a conjugate with the probe-bound target. In some embodiments, the bead is conjugated to the probe.

In some embodiments, detecting the presence or absence of the probe-bound target comprises detecting the probe-bound target or a portion thereof. In some embodiments, detecting the presence or absence of the probe-bound target comprises detecting the bead or a portion thereof. In some embodiments, detecting the presence or absence of the probe-bound target comprises detecting the labeled bead. In some embodiments, detecting the presence or absence of the probe-bound target comprises detecting an electrochemiluminescent tag. In some embodiments, the electrochemiluminescent tag comprises Tris(bipyridine)ruthenium(II) dichloride. In some embodiments, the electrochemiluminescent tag is Ruthenium (II) tris-bipyridine, N-hydroxysuccinimide. In some embodiments, detecting the presence or absence of the probe-bound target comprises detecting a SULFO TAG. In some embodiments, the detecting step comprises luminescence. In some embodiments, the detecting step comprises electrochemiluminescence.

In some embodiments, the method further comprises purification of the probe-bound target. In some embodiments, the probe-bound target is an unoccupied target. In some embodiments, the probe-bound target is a drug-occupied target. In another embodiment, purification of the probe-bound target comprises magnetic separation of probe-bound targets from non-probe-bound targets.

In some embodiments, the sample is a pre-treated sample, wherein the pre-treated sample is contacted with a drug prior to contact with the probe. In some embodiments, the sample is a non-treated sample, wherein the sample is not contacted with a drug prior to contact with the label.

In some embodiments, the probe comprises an agent. In some embodiments, the probe comprises an agent and a linker. In some embodiments, the probe comprises a label. In some embodiments, the probe comprises a label and a linker. In some embodiments, the agent is a BTK inhibitor. In some embodiments, the BTK inhibitor is a reversible BTK inhibitor. In some embodiments, the BTK inhibitor is an irreversible BTK inhibitor. In some embodiments, the BTK inhibitor is a selective, covalent BTK inhibitor. In some embodiment, the BTK inhibitor forms a covalent bond with a cysteine residue of a Bruton's tyrosine kinase (BTK). In some embodiments, the cysteine residue is cysteine 481. In some embodiments, the BTK inhibitor is selected from a list comprising LFM-A13, AVL-291, AVL-101, AVL-292, and ONO-WG-307. In some embodiments, the BTK inhibitor is ibrutinib. In some embodiments, the agent is an ITK inhibitor. In some embodiments, the agent is a BMX kinase inhibitor. In some embodiments, the agent is a TEC kinase inhibitor. In some embodiments, the agent is a BLK inhibitor.

In some embodiments, the agent is identical to the drug. For example, the drug and the agent can both be a BTK inhibitor (e.g., ibrutinib, AVL-292, ONO-WG-307). In some embodiments, the agent is similar to the drug. For example, the drug can be a BTK inhibitor and the agent can be a salt derivative of the BTK inhibitor. In some embodiments, the agent is different from the drug. For example, the drug can be ibrutinib and the agent can be AVL-292.

In some embodiments, the target is a receptor. In some embodiments, the target is a ligand. In some embodiments, the target is a kinase. In some embodiments, the kinase is BTK. In some instances, the kinase is ITK. In other embodiments, the kinase is BMX or BLK. In some instances, the kinase is TEC or TXK. In some embodiments, the kinase is HER1, HER2, HER3, or HER4. Alternatively, the kinase is JAK3.

In some embodiments, validating the drug comprises determining the efficacy of the drug on a target. In some embodiments, determining occupancy of the target by the drug comprises quantifying the presence or absence of probe-bound targets. In some embodiments, the drug is effective when the occupancy of the target is at least about 50%, 60%, 65%, 70%, 75%, 80%, 85%, 90%, 95%, 97%, or 99%.

### Diagnostics

Any of the methods, assays and systems can be used to inform therapeutic treatment and the over-all health care management of a subject informing method for determining a therapeutic regimen. In some embodiments, is a method for determining a therapeutic regimen comprising: (a) combining a sample comprising a target with a probe; (b) detecting the presence or absence of a probe-bound target; and (c) determining a therapeutic regimen based on the presence or absence of the probe-bound target.

Further disclosed herein is a method for determining efficacy of a test agent comprising: (a) combining a sample comprising a target with a probe; (b) detecting the presence or absence of a probe-bound target; and (c) determining efficacy of a test agent based on the presence or absence of the probe-bound target.

Further disclosed herein is a method for identifying drug responders comprising: (a) combining a sample comprising a target with a probe; (b) detecting the presence or absence of a probe-bound target; and (c) identifying drug responders based on the presence or absence of the probe-bound target.

Further disclosed herein is a method for identifying kinase modulators comprising: (a) combining a sample comprising a target with a probe; (b) detecting the presence or absence of a probe-bound target; and (c) identifying kinase modulators based on the presence or absence of the probe-bound target.

Disclosed herein is a method for determining drug resistance comprising: (a) combining a sample comprising a target with a probe; (b) detecting the presence or absence of a probe-bound target; and (c) determining drug resistance based on the presence or absence of the probe-bound target.

### Samples

In some embodiments, the methods, assays, and systems disclosed herein comprise contacting sample comprising a target with a probe. Suitable samples for use in any of the methods, assays, and systems disclosed herein comprise, but are not limited to, a whole blood sample, peripheral blood sample, lymph sample, tissue sample, tumor biopsy sample, bone marrow sample, or other bodily fluid sample. In some embodiments, the sample is a sample containing one or more cell types, or a lysate thereof, derived from a whole blood sample, peripheral blood sample, lymph sample, tissue sample, tumor biopsy sample, bone marrow sample, or other bodily fluid sample. Examples of bodily fluids include, but are not limited to, smears, sputum, biopsies, secretions, cerebrospinal fluid, bile, blood, lymph fluid, saliva, and urine. In some embodiments, cells of the sample are isolated from other components of the sample prior to use in the methods provided. In some embodiments, particular cell types of the sample are isolated from other cell types of the sample prior to use in the methods provided. For example, in some embodiments, peripheral blood mononuclear cells (PBMCs, e.g., lymphocytes, monocytes and macrophages) of a blood sample are isolated from other cell types of the blood sample prior to use in the methods provided. For example, in some embodiments, lymphocytes (e.g., B cells, T cells or NK cells) of the sample are isolated from other cell types of the sample prior to use in the methods provided. For example, in some embodiments, B cells of the sample are isolated from other cell types of the sample prior to use in the methods provided. In some embodiments, cells of the sample are lysed prior to use in the methods provided. For example, in some embodiments, cancer cells are isolated from normal cells of the sample prior to use in the methods provided.

Any of the samples disclosed herein comprises complex populations of cells, which can be assayed as a population, or separated into sub-populations. Such cellular and acellular samples can be separated by centrifugation, elutriation, density gradient separation, apheresis, affinity selection, panning, FACS, filtration, centrifugation with Hypaque, etc. By using antibodies specific for markers identified with particular cell types, a relatively homogeneous population of cells can be obtained. Alternatively, a heterogeneous cell population can be used.

Once a sample is obtained, it can be used directly, frozen, or maintained in appropriate culture medium for short periods of time. Methods to isolate one or more cells for use according to the methods of this invention are performed according to standard techniques and protocols well-established in the art.

In some embodiments, the sample is obtained from a subject. Such subject can be a human or a domesticated animal such as a cow, chicken, pig, horse, rabbit, dog, cat, or goat. In some embodiments, the cells used in the present invention are taken from a patient. Samples derived from an animal, e.g., human, can include, for example whole blood, sweat, tears, saliva, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid, a lavage of a tissue or organ (e.g., lung) or tissue which has been removed from organs, such as breast, lung, intestine, skin, cervix, prostate, pancreas, heart, liver and stomach.

To obtain a blood sample, any technique known in the art can be used, e.g., a syringe or other vacuum suction device. A sample can be optionally pre-treated or processed prior to enrichment. Examples of pre-treatment steps include the addition of a reagent such as a stabilizer, a preservative, a fixant, a lysing reagent, a diluent, a drug, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent. For example, when a blood sample is obtained, a preservative such an anti-coagulation agent and/or a stabilizer can be added to the sample prior to enrichment.

A sample, such as a blood sample, can be analyzed under any of the methods, assays and systems disclosed herein within 1 week, 6 days, 5 days, 4 days, 3 days, 2 days, 1 day, 12 hrs, 6 hrs, 3 hrs, 2 hrs, or 1 hr from the time the sample is obtained.

In some embodiments, a sample can be combined with an enzyme or compound that selectively lyses one or more cells or components in the sample. For example, in a blood sample, platelets and/or enucleated red blood cells are selectively lysed to generate a sample enriched in nucleated cells. The cells of interest can subsequently be separated from the sample using methods known in the art.

When obtaining a sample from a subject (e.g., blood sample), the amount can vary depending upon subject size and the condition being screened. In some embodiments, up to 50, 40, 30, 20, 10, 9, 8, 7, 6, 5, 4, 3, 2, or 1 ml, of a sample is obtained. In some embodiments, 1-50, 2-40, 3-30, or 4-20 mL of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

### Diseases and Indications

In some embodiments, any of the samples disclosed herein is obtained from a subject suffering from a disease or indication. In some embodiments, any of the samples disclosed herein is obtained from a subject suffering from a disease or indication mediated by a TEC family kinase. In some embodiments, the sample is from a subject suffering from an autoimmune disease, an inflammatory disorder, or a proliferative disease, such as cancer. In some embodiments, the cancer is a solid tumor.

In some embodiments, the sample is from a subject suffering from a cancer. Cancers include, but are not limited to, sarcomas, carcinomas, and hematologic cancers. In some embodiments, the hematologic cancer is a leukemia, a lymphoma, or a myeloma.

In some embodiments, the cancer is a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g., alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

In some embodiments, the cancer is a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. In some embodiments, the cancer is a pancreatic cancer, colon cancer, breast cancer, lung cancer, ovarian cancer, prostate cancer, thyroid cancer, bladder cancer, or proximal or distal bile duct carcinoma. In some embodiments, the cancer is a breast cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesothelioma. Examples of NSCLC include squamous cell carcinoma, adenocarcinoma, and large cell carcinoma. In some embodiments, the mesothelioma is a cancerous tumor of the lining of the lung and chest cavity (pleura) or lining of the abdomen (peritoneum). In some instances, the cancer is a brain cancer, such as a glioblastoma.

In some embodiments, the cancer is a central nervous system (CNS) tumor. CNS tumors can be classified as gliomas or nongliomas. In some instances, the glioma is malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

In some instances, the cancer is a leukemia. In some instances, the leukemia is an acute lymphocytic leukemia, acute lymphoblastic leukemia (ALL), precursor B-cell lymphoblastic leukemia, acute myelocytic leukemia (AML), acute promyelocytic leukemia (APL), chronic lymphocytic leukemia (CLL), chronic myelocytic leukemia (CML) or acute monocytic leukemia (AMoL). Additional types of leukemias include, but are not limited to hairy cell leukemia, chronic myelomonocytic leukemia, and juvenile myelomonocytic-leukemia.

In some instances, the cancer is a lymphoma. In some instances, the lymphoma is a Hodgkin's lymphoma. In other instances, the lymphoma is a non-Hodgkin's lymphoma (NHL). In some embodiments, the lymphoma is a B-cell NHL. A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, precursor B-cell lymphoblastic lymphoma, primary central nervous system (CNS) lymphoma, and AIDS-related lymphoma. Additional non-Hodgkin's lymphomas are contemplated within the scope of the present invention and apparent to those of ordinary skill in the art.

In some embodiments, the cancer is a T cell lymphoma. In some embodiments, the T cell lymphoma is extranodal T cell lymphoma, cutaneous T cell lymphomas (CTCL), peripheral T-cell lymphoma (PTCL), Sézary syndrome, mycosis fungoides, anaplastic large cell lymphoma, or angioimmunoblastic T cell lymphoma.

In some embodiments, the subject is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In other embodiments, the subject is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In some embodiments, the subject has an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In further embodiments, the subject is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In some embodiments, the subject is administered or has been administered one or more therapeutic agents for treatment of a disease or condition. In some embodiments, the subject is administered or has been administered a BTK inhibitor for treatment of a disease or condition. In some embodiments, the subject is administered or has been administered one or more therapeutic agents in addition to a BTK inhibitor for treatment of a disease or condition.

In some embodiments, the subject is administered or has been administered one or more chemotherapeutic agents for treatment of cancer. In some embodiments, the subject is administered or has been administered a BTK inhibitor for treatment of a cancer. In some embodiments, the subject is administered or has been administered one or more chemotherapeutic agents in addition to a BTK inhibitor for treatment of cancer. In some embodiments, the BTK inhibitor is ibrutinib.

### Additional Features of Methods, Assays, and Systems

Disclosed herein are additional features of the methods, assays, and systems disclosed herein. The efficacy of the methods, assays and systems disclosed herein are about comparable to current protein occupancy methods, assays and systems (e.g., gel-based assays). In some embodiments, the efficacy of the methods, assays and systems are better than the current protein occupancy methods. In some instances, the methods, assays and systems provide improved specificity as compared to current protein occupancy methods. For example, the assay offers good specificity when signals for the negative control Jurkat lysates labeled with probe were at background levels for all lysate concentrations tested. In some instances, the specificity is at least about 50%, 55%, 60%, 65%, 70%, 75%, 80%, 85%, 90%, 95%, 97%, or 99%.

In some instances, the methods, assays, and systems disclosed herein provide improved sensitivity. In some instances, improved sensitivity can be determined from the amount of sample required. In some instances, the methods, assays, and systems allows for the use of at least about 2 times less lysate, at least about 3 times less lysate, at least about 4 times less lysate, at least about 5 times less lysate, at least about 6 times less lysate, at least about 7 times less lysate, at least about 8 times less lysate, at least about 9 times less lysate, or at least about 10 times less lysate than current methods. In some embodiments, about 2-10 times less lysate, about 3-7 times less lysate, about 3-6 times less lysate is used than current methods. For example, the improved sensitivity of the assay allows for use of 3-5 times less lysate than Western blot/ELISA, and to conserve precious samples.

The methods, assays and systems disclosed herein are straight-forward. The methods, assays and systems disclosed herein are quicker than current methods (e.g., Western blot). The methods, assays and systems disclosed herein can be completed in less than about 10 hours, less than about 8 hours, less than about 7 hours, less than about 6 hours, less than about 5 hours, less than about 4 hours. Preferably, methods, assays and systems disclosed herein can be completed in less than about 2-7 hours, less than about 3-6 hours, less than about 3-5 hours.

The methods, assays and systems disclosed herein also provide for increased throughput. In some embodiments, throughput is increased by at least about 5%, at least about 10%, at least about 15%, at least about 20%, at least about 25%, at least about 30%, at least about 40%, at least about 50%, or at least about 60%.

The methods, assays and systems disclosed herein also provide for more uniform test conditions. For example, more samples can be run on a plate-based assay than on a single gel. Therefore, samples on a single plate will have uniform test conditions than samples on multiple gels. Furthermore, the uniformity of multiple plate-based assays is greater than the uniformity of multiple gels. In some instances, the uniformity of test conditions is at least about 20%, at least about 30%, at least about 40%, at least about 50%, at least about 60%, at least about 70%, at least about 80%, or at least about 90% greater.

In some embodiments, the methods, assays, and systems disclosed herein enable the use of less probe than current methods (e.g., gel-based formats). In some embodiments, at least less than about 10×, 20×, 30×, 40×, 50×, 60×, 70× less probe is used than current methods (e.g., gel-based formats). For example, the assay requires 40× less probe than the gel based format thereby offering savings on reagents.

In some embodiments, the methods, assays, and systems disclosed herein provide a large signal window. In some embodiments, the large signal window comprises 50:1, 60:1, 70:1, 80:1, 90:1, 100:1, 200:1, 300:1, 400:1, 500:1, 600:1, 700:1, 800:1, 900:1, 1000:1 with 9 μg per well of lysate,

In some embodiments, the methods, assays, and systems disclosed herein provide good reproducibility. In some embodiments, reproducibility is about less than about 10% CV, less than about 8% CV, less than about 6% CV, less than about 5% CV, less than about 4% CV, less than about 3% CV. Preferably, reproducibility is less than about 4-6% CV.

Techniques and suggestions to optimize the methods, assays, and systems disclosed herein include, but are not limited to imaging the plates in sectors, energizing the sectors once, and avoiding introducing bubbles in samples, detection antibody and read buffer solution. Further ways to optimize the methods, assays, and systems disclosed herein comprise keeping detection antibody stocks in the dark. However, working solutions do not need to be shielded from light. Furthermore, when adding small volumes, in the order of 25 μL, add to the bottom corner of the wells. In some embodiments, the methods, assays, and systems disclosed herein comprise shaking the plates during blocking, sample incubation, detection antibody incubation. For optimization, do not leave assays in wash buffer or read buffer for extended periods of time. However, if additional time is required, leave in sample or detection antibody. Preferably, polypropylene plates and tubes are used for sample handling. Preferably, polystyrene for sample handling should be avoided. In some embodiments, samples can be vortexed and/or centrifuged prior to contact with the probe. Vortexing and/or centrifugation of the sample can ensure removal of any debris in sample. In some instances, plates can be blocked overnight at 4° C. In some instances, plates can be blocked for 1 hour at room temperature. If blocking overnight, allow the plate to equilibrate to room temp before proceeding with the assay.

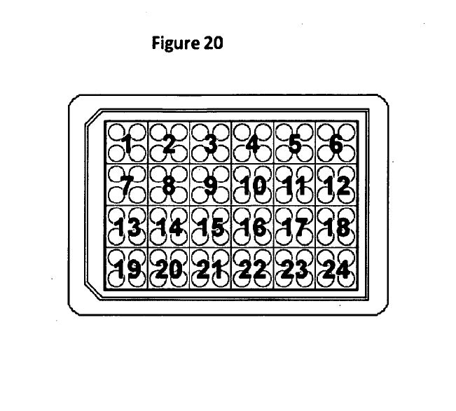

In some embodiments, the methods, assays and systems disclosed herein comprise the use of a partial plate. In some instances, instructions for partial plate usage can be provided. In some instances, the methods, assays and systems disclosed herein comprise allowing plates to equilibrate to room temperature before opening the plate package. For the SI2400 MSD SECTOR IMAGER, the MSD plate is divided into 24 sectors (2×2=4 wells/sector—see FIG. 20). Sectors can be energized once and as such when using partial plates, samples should be allocated to sectors. In some instances, volumes for all reagents are adjusted based on the portion of plate used. In some instances, unused sectors should be covered with a plate seal and kept dry during the course of the assay. In some instances, a plate seal should be removed prior to reading the plate.

### EXAMPLES

### Example 1

### Comparison of an Anti-Protein Antibody Coated Plate and a Streptavidin Coated Plate Assay Formats

The aim of the study was to convert an existing gel-based BTK occupancy assay to a plate based electrochemiluminescent assay to increase assay throughput. The purpose of the assay is to determine the relative amount of BTK that has not been bound by the covalent inhibitor (ibrutinib) hereafter referred to as the “drug” or ibrutinib. The drug binds to the active site of BTK and forms a disulfide bond with a cysteine residue. Compound I-5 hereafter referred to as the “probe” consists of the ibrutinib linked to biotin via a long chain linker. In the gel-based assay, the probe is labeled with a fluorescent reporter. Labeling of samples labeled by the probe allows for the detection of BTK not occupied by drug. Two potential assay formats are shown in FIG. 11.

### Experiment 1

In this study, assay formats were tested for sensitivity, specificity, range, and to determine most suitable anti BTK Antibody.

Samples

Positive Control:

An aliquot of DOHH2 cell lysate (1 mg/mL) was inhibited with 1 μM ibrutinib then labeled with probe (1 μM).

Negative Controls:

Untreated DOHH2 and Jurkat cell lysates (1 mg/mL) were labeled with probe (1 μM) and Untreated DOHH2 cell lysate

Materials Used:

Standard Streptavidin plate (5-pack) Catalog #L15SA-2; Read Buffer T (50 mL) Catalog # R92TC-3, SULFO TAG Goat Anti-mouse (50 μg) Catalog# R32AC-5; SULFO TAG Goat Anti-rabbit (50 μg) Catalog# R32AB-5; SULFO TAG streptavidin (50 ug) Catalog# R32AD-5; MSD Standard Plates Catalog # L15XA-3; MSD Blocker A Catalog# R93AA-2; Protease inhibitor cocktail (e.g., Thermo/Pierce Halt™ Protease Inhibitor Cocktail EDTA free Cat #87785 or Roche Complete Mini Protease inhibitor tablet, Cat #1836170); Positive Control lysates from BTK expressing cell line (DOHH2); Negative Control lysates (Jurkat); ibrutinib (PCI); probe compound I-5 (biotinylated probe)

BTK Antibodies:

The following antibodies were tested:

| | Species | Cat# | Vendor | Buffer | Conc. | |
|---|---|---|---|---|---|---|
| a-BTK | Mouse | 611116 | BD | BSA, | 250 | μg/mL |
| | | | | glycerol, | | |
| | | | | azide | | |
| a-BTK | Mouse | 611117 | BD | BSA, | 250 | μg/mL |
| | | | | glycerol, | | |
| | | | | azide | | |
| a-BTK | Rabbit | SAB4502936 | Sigma | unknown | 1 | mg/mL |

Solutions:

Blocking Solution:

3% (w/v) MSD Blocker A in 1× Tris Wash buffer: 3 g Blocker A+100 mL 1× Tris wash Buffer. Store at 4° C. for up to 14 days. Blocking solution can also be prepared in PBS-T

Wash Buffer:

1×MSD Tris wash buffer: 50 mL 10× Tris Wash buffer+450 mL H₂O (150 mM NaCl 50 mM Tris-HCl pH 7.5 0.02% Tween-20). PBS-T can also be used as a wash buffer

Capture Antibody Dilution Buffer:

PBS w/o Ca2+w/o Mg2+

Detection Antibody Dilution Buffer:

1% MSD Blocker A in 1× Tris Wash Buffer: 10 mL Blocking solution+20 mL 1× Tris Wash Buffer or 10 mL Blocking solution+20 mL PBS-T

Read Buffer:

1×MSD Read Buffer T: per plate 5 mL 4× Read Buffer T+15 mL H2O; 2×MSD Read Buffer T: per plate 10 mL 4× Read Buffer T+10 mL H₂O

Cell Lysates:

Lysates were prepared by repeated freeze-thaw of cell pellets resuspended in PBS+ protease inhibitors. PCI and probe labeling reactions were carried out in PBS-T+1% BSA (assay buffer). Lysates were diluted in assay buffer+ protease inhibitors.

Format 1: Streptavidin Detection

FIG. 11A is a schematic of the Streptavidin detection assay (e.g., Assay Format 1). Briefly, the method comprises contacting a drug-treated sample comprising a target (e.g., a BTK kinase) with an anti-BTK antibody coated plated, wherein the BTK kinase is captured by the anti-BTK antibody. The BTK kinases captured by the anti-BTK antibody include drug-occupied BTK kinases and unoccupied BTK kinases. The captured BTK kinases are contacted with a probe. The probe comprises an agent that binds to the unoccupied BTK kinases. In this example, the probe is unable to bind to drug-occupied BTK kinases. The probe binds to the unoccupied BTK kinases to found a probe-bound BTK kinase. In this example, the probe comprises a label, which enables detection of the probe-bound BTK kinases. The probe-bound BTK kinases are detected by the addition of a labeled bead (e.g., SULFO TAG Streptavidin). The amount of probe-bound kinases is quantified by electrochemiluminescence. The quantification of the probe-bound kinases enables the determination of occupancy of the BTK kinase and efficacy of the drug. A more detailed protocol is disclosed herein.

Protocol—Assay Format 1

1. Coat MSD Standard plate with 30 μL per well of anti-BTK capture antibody solution (diluted to 2 μg/mL in PBS). After addition of the Ab solution to the bottom corner of the wells, tap the plate to ensure that the solution distributes evenly across the bottom of each well. Seal with an adhesive plate seal and incubate O/N at 4° C. Do not shake the plate.

2. Tap out plate and add 150 μL per well blocking solution (3% [w/v] Blocker A). Seal and incubate with shaking 1 h or more at RT.

3. Wash plate 1× with ≧150 μL per well wash buffer. Tap dry.

4. Add 30 μL of lysate according to the plate layout. Add lysate solution to the bottom corner of the wells. Seal and shake plate at 300-500 rpm 1 h at RT.

5. Wash plate 3× with ≧150 μL per well wash buffer. Tap dry.

6. Add 25 μL per well of SULFO TAG streptavidin diluted to 0.5 μg/mL in 1% (w/v) Blocker A according to the plate layout. Seal and shake plate at 300-500 rpm 30-60 min at RT.

7. Wash plate 3× with ≧150 μL per well 1× Tris wash buffer. Tap dry

8. Add 150 μL per well 1× Read Buffer T (diluted in H20). Avoid bubbles—use reverse pipetting when adding Read Buffer. Read plate in SI2400

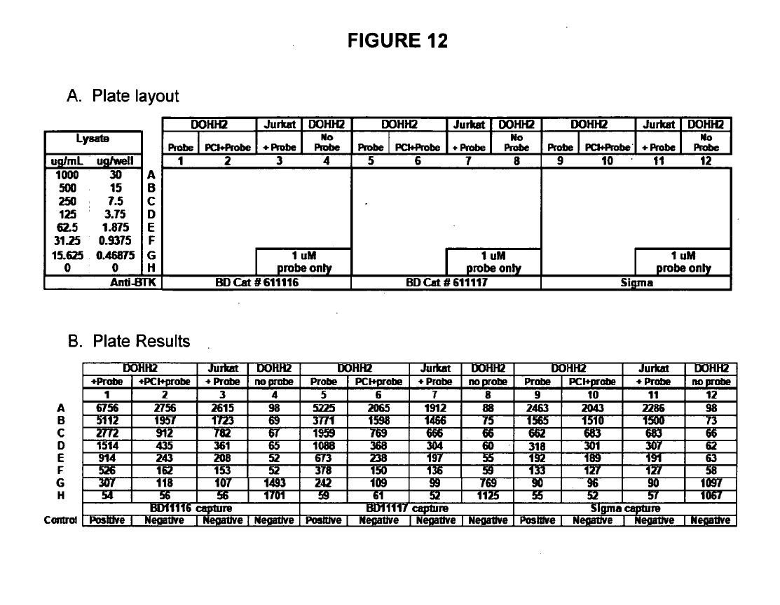

The plate layout and results are shown in FIG. 12.

The signal of the positive control (DOHH2+probe) titrates with the lysate concentration for all three capture antibodies tested. The highest signals were obtained using BD611116 and BD611117 anti-BTK capture antibodies. The BD611116 and BD611117 anti-BTK capture antibodies were comparable.

A maximum signal:background ratio for the positive control (DOHH2+probe) of 125:1 was obtained with 1 μg/μL positive control lysate using the BD61116 anti-BTK capture antibody.

Background was low for all concentrations of the DOHH2 (no probe) lysate tested indicating that there is no non-specific binding of SULFO TAG streptavidin to lysate proteins.

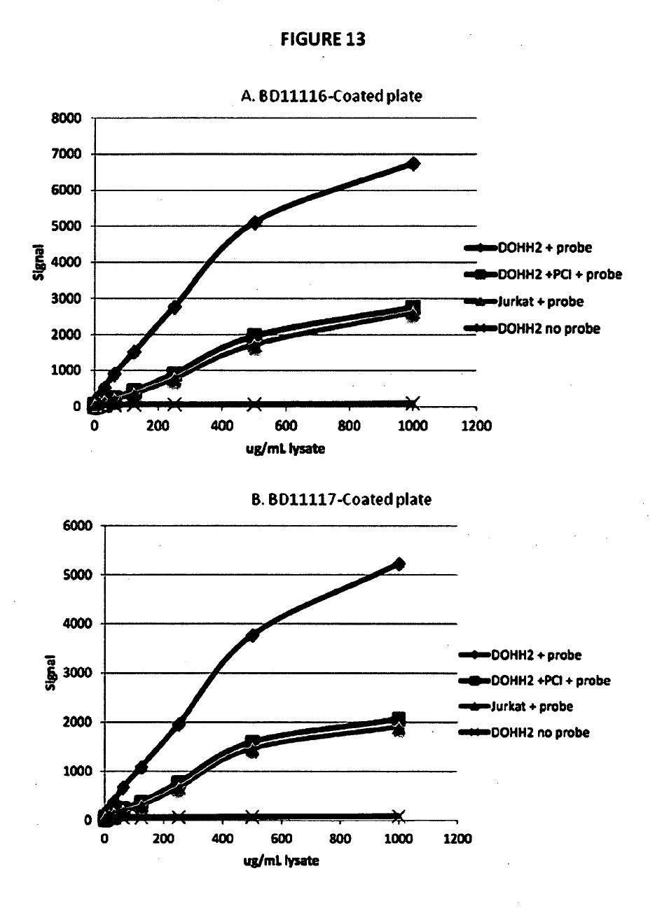

The positive control (DOHH2+probe) lysate signals can be differentiated from the negative controls (DOHH2+PCI+probe) and (Jurkat+probe) when using BD 611116 (see FIG. 13A) and BD611117 (see FIG. 13B) anti-BTK capture antibodies but not when using the Sigma anti-BTK as capture antibody suggesting that the Sigma anti-BTK can bind other proteins in the cell lysates labeled by probe. The specificity ratios of positive:negative controls are shown in Table 1.

TABLE 1
Specificity ratios of positive:negative controls

| Ratio | DOHH2 + Probe:Probe + PCI | | | Ratio | DOHH2 + Probe:Jurkat + probe | | |
|---|---|---|---|---|---|---|---|
| Lysate | Capture AB | | | Lysate | Capture AB | | |
| ug/ml | BD11116 | BD1117 | Sigma | ug/ml | BD11116 | BD1117 | Sigma |
| 1000 | 2.45 | 2.53 | 1.21 | 1000 | 2.58 | 2.73 | 1.08 |
| 500 | 2.61 | 2.36 | 1.04 | 500 | 2.97 | 2.57 | 1.04 |
| 250 | 3.04 | 2.55 | 0.97 | 250 | 3.54 | 2.94 | 0.97 |
| 125 | 3.48 | 2.96 | 1.06 | 125 | 4.19 | 3.58 | 1.04 |
| 62.5 | 3.76 | 2.83 | 1.02 | 62.5 | 4.39 | 3.42 | 1.01 |
| 31.25 | 3.25 | 2.52 | 1.05 | 31.25 | 3.44 | 2.78 | 1.05 |
| 15.63 | 2.60 | 2.22 | 0.94 | 15.63 | 2.87 | 2.44 | 1.00 |
| 0 | 0.96 | 0.97 | 1.06 | 0 | 0.96 | 1.13 | 0.96 |

The Sigma anti-BTK antibody was not used in follow up experiments.

Format 2: Streptavidin Capture

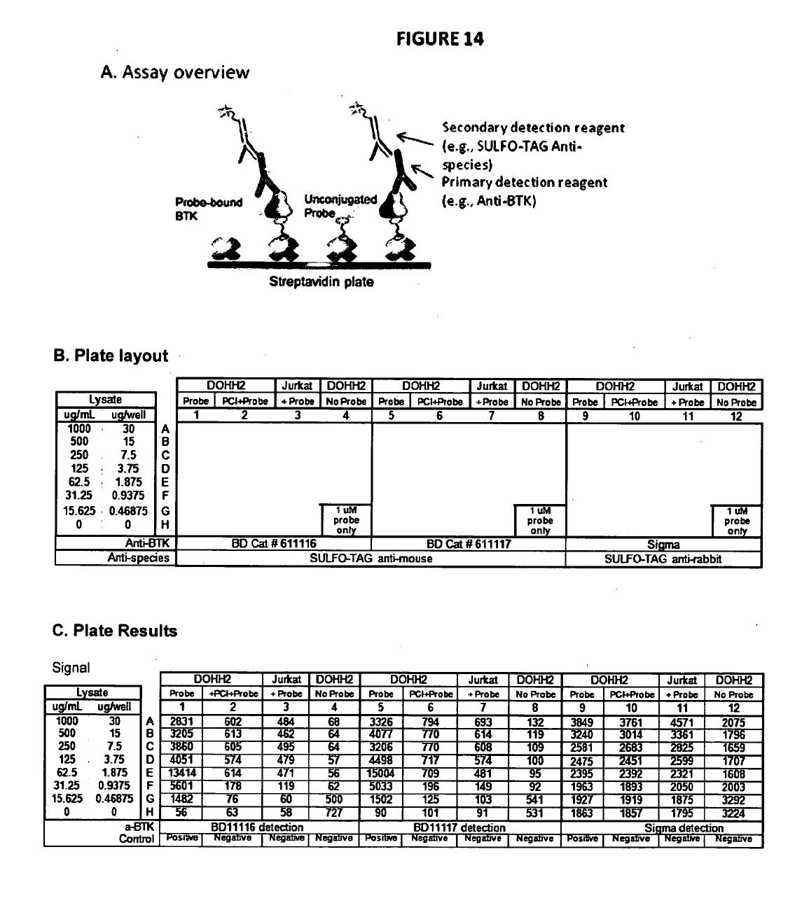

FIGS. 11B and 14A show schematics of the streptavidin-coated plate assay (e.g., Assay Format 2). Briefly, the method comprises blocking a MSD standard streptavidin plate with a probe or providing a probe-bound plate. In this example, the probe comprises a label and an agent, wherein the label (e.g., biotin) is captured by the streptavidin, thereby attaching the probe to the plate. A sample comprising a target (e.g., BTK) is applied to the plate. The target binds to or attaches to the probe via the agent (e.g., a drug such as a BTK inhibitor) to form a probe-bound compound. The probe-bound targets can be detected by a primary detection agent such as anti-target (e.g., anti-BTK) antibody. The primary detection reagent can be labeled and subsequently directly detected. As shown in FIG. 11B, the primary detection reagent is conjugated to a SULFO TAG to form a labeled primary detection reagent. However, as shown in FIG. 14A, the primary detection reagent can be unlabeled. The method can further comprise the addition of a secondary detection agent (e.g., anti-species antibody), as shown in FIG. 14A. The secondary detection agent can be labeled (e.g., SULFO TAG anti-species antibody) and subsequently detected. The SULFO-labeled detection agents can be detected by electrochemiluminescence, which enables quantification of the probe-bound kinases. The quantification of the probe-bound kinases enables the determination of occupancy of the BTK kinase and efficacy of the drug. A more detailed protocol is disclosed herein.

Protocol—Assay Format 2

1. Add 150 μL per well blocking solution (3% [w/v] Blocker A) to MSD standard streptavidin plate. Seal and incubate with shaking 1 h or more at RT or block overnight at 4° C.

2. Wash plate 1× with ≧150 μL per well wash buffer. Tap dry.

3. Add 30 μL of lysate according to the plate layout. Add lysate solution to the bottom corner of the wells. Seal and shake plate at 300-500 rpm 1 h at RT.

4. Wash plate 3× with ≧150 μL per well wash buffer. Tap dry.

5. Add 25 μL per well of anti-BTK antibody diluted to 0.5 μg/mL in 1% (w/v) Blocker A according to the plate layout. Seal and shake 1 h at RT.

6. Wash plate 3× with ≧150 μL per well wash buffer. Tap dry. Add 25 μL per well of SULFO TAG conjugated anti-species antibody diluted to 1 μg/mL in 1% (w/v) Blocker A according to the plate layout. Seal and shake plate 1 h at RT.

7. Wash plate 3× with ≧150 μL per well 1× Tris wash buffer. Tap dry

8. Add 150 μL per well 1× Read Buffer T (diluted in H20). Avoid bubbles—use reverse pipetting when adding Read Buffer. Read plate in SI2400

### Results

The plate layout and results are shown in FIG. 14B-C.

The signal of the positive control (DOHH2+probe) titrates with the lysate concentration when using BD611116 and BD611117 anti-BTK as detection antibodies up to 62 μg/mL lysate after which there is a hook effect possibly due to the presence of excess probe competing with probe-labeled BTK for the streptavidin surface. The hook occurs when final probe concentration in the sample is >62 nM.

A maximum signal:background ratio for the positive control (DOHH2+probe) of 240:1 was obtained with 0.06 μg/μL positive control lysate using the BD61116 anti-BTK detection antibody.

Background was low for all concentrations of the DOHH2 (no probe) lysate tested when using BD611116 and BD611117 anti-BTK as detection antibodies indicating that there is no non-specific binding of these antibodies to lysate proteins. In contrast, the Sigma anti-BTK antibody yielded high background signals for all conditions tested.

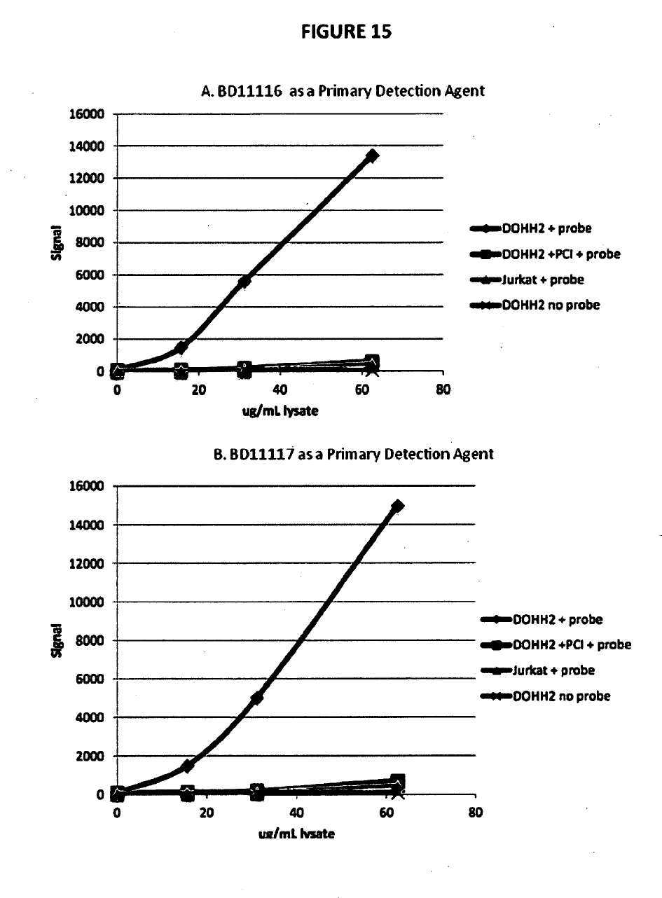

The positive control (DOHH2+probe) lysate signals can be differentiated from the negative controls (DOHH2+PCI+probe) and (Jurkat+probe) when using BD 611116 (see FIG. 15A) and BD611117(see FIG. 15B) anti-BTK detection antibodies but not when using the Sigma anti-BTK as detection antibody suggesting that the Sigma anti-BTK can bind other proteins in the cell lysates labeled by probe. Specificity ratios of positive:negative controls are shown in Table 2.

TABLE 2
Specificity ratios of positive:negative controls

| Ratio | DOHH2 + Probe:Probe + PCI | | | Ratio | DOHH2 + Probe:Jurkat + probe | | |
|---|---|---|---|---|---|---|---|
| Lysate | Detection AB | | | Lysate | Detection AB | | |
| ug/ml | BD11116 | BD1117 | Sigma | ug/ml | BD11116 | BD1117 | Sigma |
| 62.5 | 21.8 | 21.2 | 1.0 | 62.5 | 28.5 | 31.2 | 1.0 |
| 31.3 | 31.5 | 25.7 | 1.0 | 31.3 | 47.1 | 33.8 | 1.0 |
| 15.6 | 19.5 | 12.0 | 1.0 | 15.6 | 24.7 | 14.6 | 1.0 |
| 0.0 | 0.9 | 0.9 | 1.0 | 0.0 | 1.0 | 1.0 | 1.0 |

The Sigma anti-BTK antibody was not used in follow up experiments.

Signals for the negative controls are comparable to the signals from the 1 μM probe only wells.

The BD611116 and BD611117 anti-BTK detection antibodies were comparable.

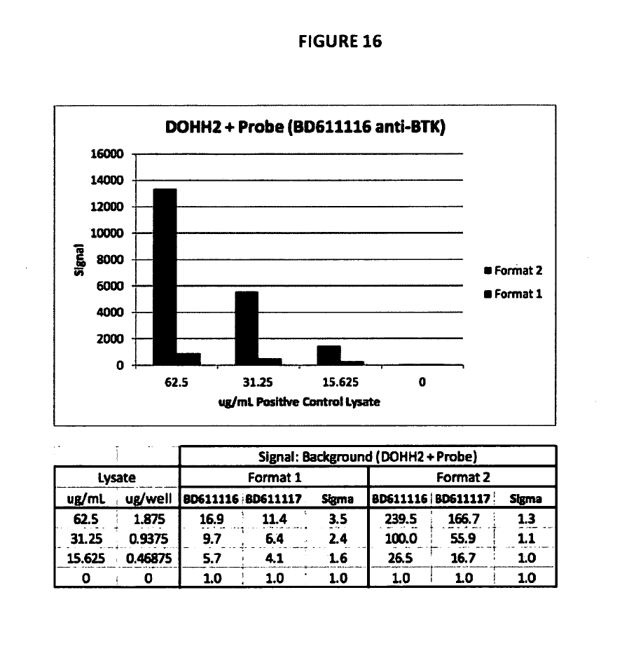

FIG. 16 shows a comparison of Assay format 1 (streptavidin-detection method) and Assay format 2 (streptavidin-capture method). As shown in FIG. 16, Assay format 2 affords better sensitivity and better specificity than assay format.

### Experiment 2

### Optimizing Probe Concentration

Assay format 2 (e.g., streptavidin-coated plate, streptavidin-capture method—see FIG. 14A) was used for further optimization. Since a hook effect was observed at probe concentration >62 nM and lysate concentration >62 μg/mL, a checkerboard experiment was carried out to determine if the hook effect is linked to excess probe or excess protein concentration. Excess unconjugated probe can compete with BTK-bound probe for binding to the streptavidin surface once the binding capacity of the plate is reached.

Aims:

Determine optimal probe concentration required

Compare different probe: lysate ratios

Samples

Positive control: prepare DOHH2 lysate at 1 mg/mL in assay buffer.

Negative control: treat an aliquot of 1 mg/mL DOHH2 lysate with 1 μM PCI to have an excess of PCI so that BTK is maximally bound by PCI.

Dilute DOHH2 only and DOHH2+PCI lysates to 100; 50; 25; 12.5; 6.25 and 3.12 μg/mL in assay buffer

Dilute probe to 10000; 2500; 625; 156; 39; 9 and 2 nM (100× concentration)

Add 1 μL 100× probe to 100 μL diluted lysates in a 96 well polypropylene plate, incubate

Protocol

1. Block MSD standard streptavidin plate with 150 μL per well 3% Blocker A 1 h at RT or overnight at 4° C.

2. Wash plate 1× with ≧150 μL per well wash buffer. Tap dry.

3. Add 50 μL of lysate according to the plate layout. Add lysate solution to the bottom corner of the wells. Seal and shake plate at 300-500 rpm 1 h at RT.

4. Wash plate 3× with ≧150 μL per well wash buffer. Tap dry.

5. Add 25 μL per well of BD611117 anti-BTK antibody diluted to 0.5 μg/mL in 1% (w/v) Blocker A. Seal and shake 1 h at RT.

6. Wash plate 3× with ≧150 μL per well wash buffer. Tap dry.

7. Add 25 μL per well of SULFO TAG conjugated anti-mouse antibody diluted to 1 μg/mL in 1% (w/v) Blocker A. Seal and shake plate 1 h at RT.

8. Wash plate 3× with ≧150 μL per well 1× Tris wash buffer. Tap dry

9. Add 150 μL per well 2× Read Buffer T (diluted in H20). Avoid bubbles—use reverse pipetting when adding Read Buffer. Read plate in SI2400

Results:

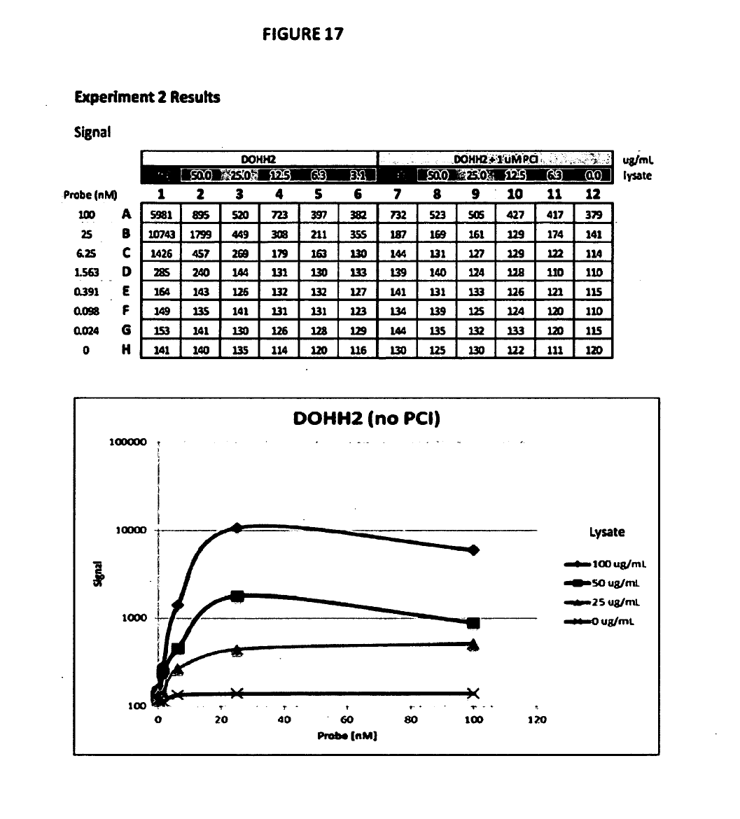

Results for the optimization experiment are shown in FIG. 17.

Backgrounds are low for the negative control (DOHH2+1 μM PCI) samples up to 25 nM probe.

Above 25 nM probe, backgrounds increase. One possible explanation for this phenomenon is signal contribution from other proteins in the lysate also labeled with probe at high probe concentration as seen in the gel-based assay and/or signal contribution from the probe alone.

For the positive control (untreated DOHH2), signals increase with increasing probe concentration up to 25 nM, above which signals decrease or plateau when there is an excess of probe.

A final probe concentration of 25 nM is recommended.

### Experiment 3

### Inhibition with Drug Titration

Aim:

Carry out inhibition experiment with titration series of PCI then label lysates with a final probe concentration of 25 nM probe.

Samples:

Positive control: DOHH2 lysate at 1 mg/mL in assay buffer.

Negative control: Jurkat lysate at 1 mg/mL in assay buffer.

Dilute lysates to 300; 150 and 75 mg/mL in assay buffer

Prepare a dilution series of PCI. The 100×PCI concentrations are: 100; 25; 6.25; 1.56; 0.39; 0.09 and 0.02 μM.

Treat DOHH2 lysates with PCI in a polypropylene plate e.g., 100 μL lysate+1 μL 100×PCI solution

After the PCI inhibition add probe to all the samples to a final concentration of 25 nM (2 μL of 1.25 μM probe stock+100 μL sample), incubate with shaking.

Protocol:

1. Block MSD standard streptavidin plate with 150 μL per well 3% Blocker A 1 h at RT or overnight at 4° C.

2. Wash plate 1× with ≧150 μL per well wash buffer. Tap dry.

3. Add 30 μL of lysate according to the plate layout. Add lysate solution to the bottom corner of the wells. Seal and shake plate 1 h at RT.

4. Wash plate 3× with ≧150 μL per well wash buffer. Tap dry.

5. Add 25 μL per well of BD611117 anti-BTK antibody diluted to 0.5 μg/mL in 1% (w/v) Blocker A. Seal and shake 1 h at RT.

6. Wash plate 3× with ≧150 μL per well wash buffer. Tap dry. Add 25 μL per well of SULFO TAG conjugated anti-mouse antibody diluted to 1 μg/mL in 1% (w/v) Blocker A. Seal and shake plate 1 h at RT.

7. Wash plate 3× with ≧150 μL per well 1× Tris wash buffer. Tap dry

8. Add 150 μL per well 1× Read Buffer T (diluted in H20). Avoid bubbles—use reverse pipetting when adding Read Buffer. Read plate in SI2400.

Results:

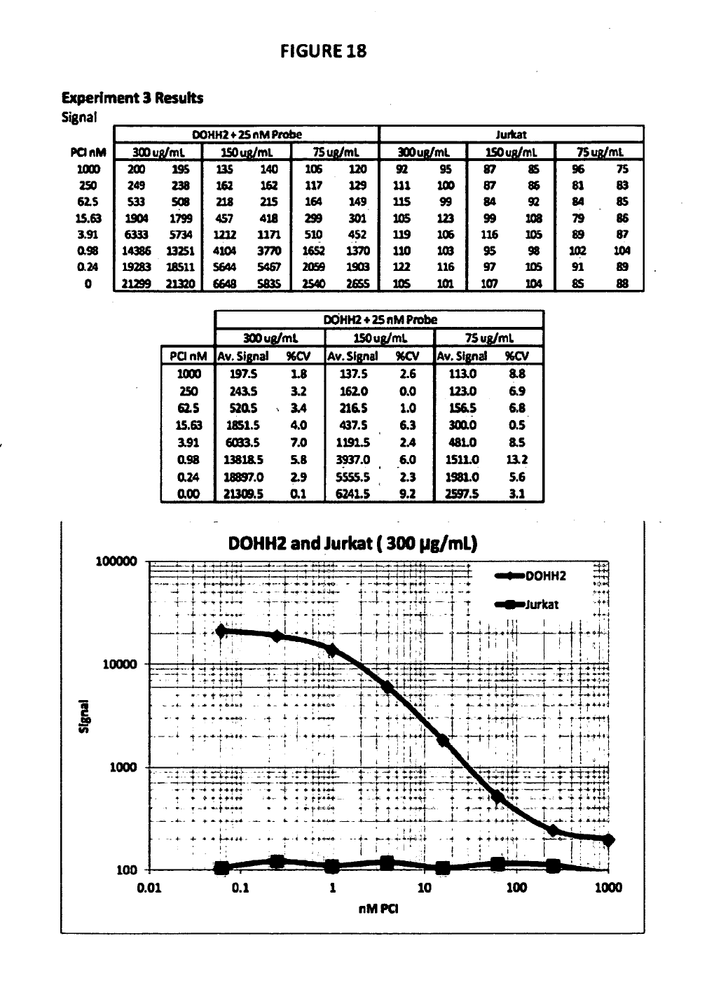

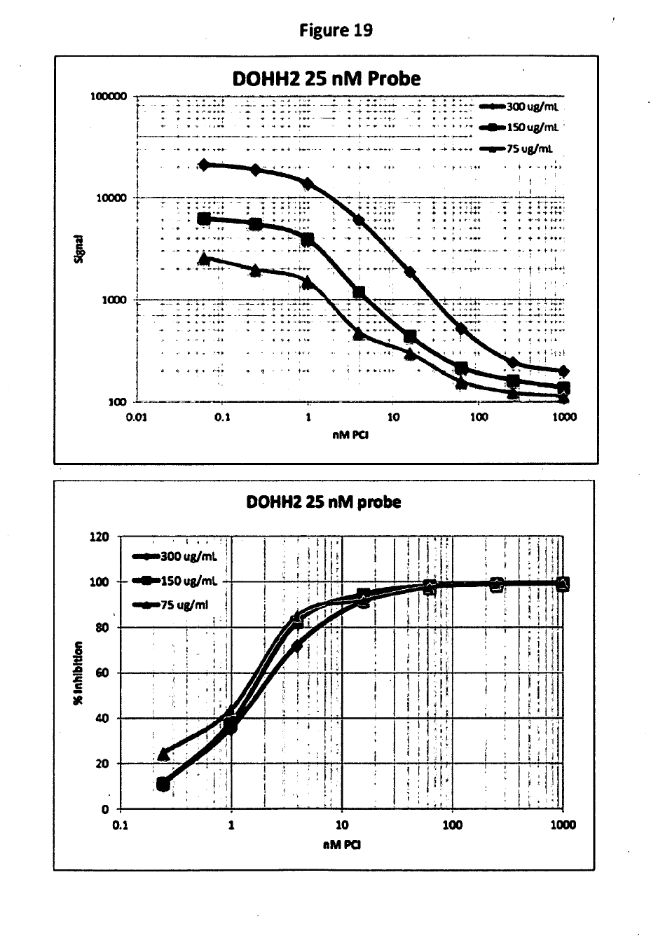

Results are shown in FIGS. 18-19

Signals of the negative control Jurkat lysates labeled with probe remained at background levels for all conditions tested.

A dose dependent decrease in signal was observed for positive control DOHH2 lysates treated with a titration series of PCI then labeled with 25 nM probe.

The PCI inhibition profile was comparable for all three concentrations of DOHH2 lysates tested (300, 150 and 75 μg/mL) and comparable to the reference gel based assay.

The assay offers a large signal window at all lysate concentrations tested:

TABLE 3
Signal ratio of 0:1000 nM PCI

| DOHH2 Lysate | Signal Window |
|---|---|
| ug/well | ug/mL |

| | | |
|---|---|---|
| 9 | 300 | 108 |
| 4.5 | 150 | 45 |
| 2.25 | 75 | 23 |

Assay reproducibility was very good with average % CV<5%. The assay allowed for high specificity (good distinction from negative controls), sensitivity (use of less lysate compared to western blot methods (0.6 μg vs. 50 μg)) and efficiency (required less probe than the gel based formats (25 nM vs. 2.5 μM)).

### Example 2

### Protein Occupancy Assay Protocol

An alternative protein occupancy assay protocol is provided.

1. Add probe to the samples in assay buffer to a final concentration of 25 nM (2 μL of 1.25 μM probe stock+100 μL sample), incubate with shaking

2. Block MSD standard streptavidin plate with 150 μL per well 3% Blocker A 1 h at RT or overnight at 4° C.

3. Wash plate 1× with ≧150 μL per well wash buffer. Tap dry.

4. Add 30 μL of lysate per well. Add lysate solution to the bottom corner of the wells. Seal and shake plate 1 h at RT.

5. Wash plate 3× with ≧150 μL per well wash buffer. Tap dry.

6. Add 25 μL per well of BD611117 or BD611116 anti-BTK antibody diluted to 0.5 μg/mL in 1% (w/v) Blocker A. Seal and shake 1 h at RT.

7. Wash plate 3× with ≧150 μL per well wash buffer. Tap dry. Add 25 μL per well of SULFO TAG conjugated anti-mouse antibody diluted to 1 μg/mL in 1% (w/v) Blocker A. Seal and shake plate 1 h at RT.

8. Wash plate 3× with ≧150 μL per well 1× Tris wash buffer. Tap dry

9. Add 150 μL per well 1× Read Buffer T (diluted in H20). Avoid bubbles—use reverse pipetting when adding Read Buffer. Read plate in SI2400.

If desired, the following parameters can further be optimized (e.g., concentration of the anti-BTK detection antibody, concentration of the MSD SULFO TAG anti-mouse secondary detection antibody, or combining the anti-BTK detection antibody and the MSD SULFO TAG anti-mouse secondary detection antibody in a single incubation step).

### Example 3

### Comparison of Selected Biotinylated Probes in Anti-Protein Antibody Coated Plate and a Streptavidin Coated Plate Assay Formats

Selected biotinylated probes were tested for use in target occupancy assays using either the anti-protein antibody coated plate or streptavidin coated plate assay formats. The following probes were tested:

Compound I-1, MW=808.01 (total—80 mg)

Compound I-2, MW=793.38 (total—60 mg)

Compound I-3, MW=780.98 (total—32 mg)

Compound I-4, MW=766.95 (total—27 mg)

Compound I-5, MW=1097.37 (total—24 mg)

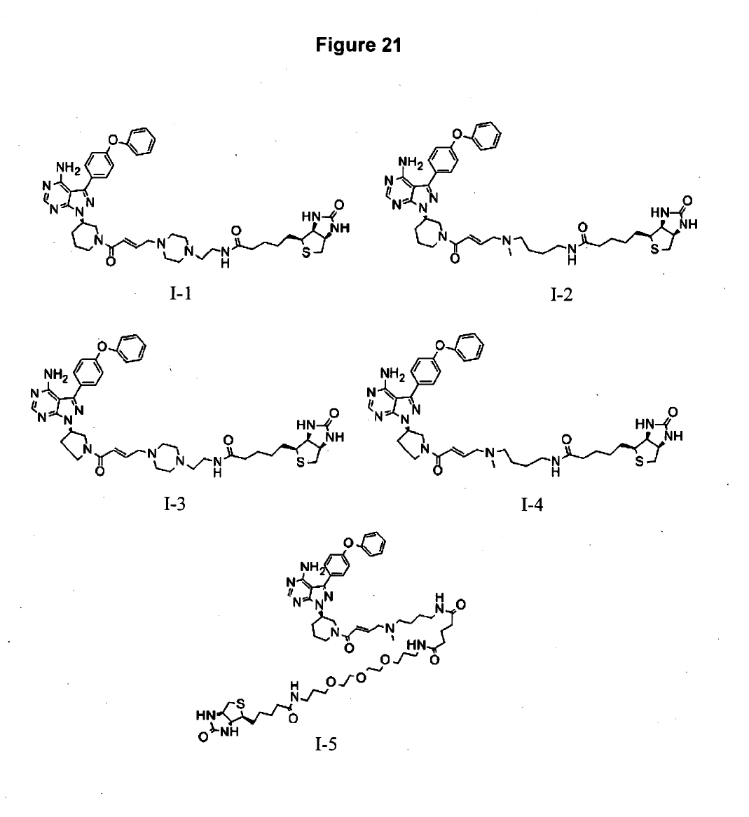

The structures of the probes tested are shown in FIG. 21.

Stock solutions (5 mM and 10 mM) of the probes were prepared as follows. Compound I-1, I-2, I-3, and I-4: 2 or 4 mg probe in 500 μl DMSO and Compound I-5: 12 or 24 mg in 2.2 ml DMSO, aliquots in 100 μl. For the assays 50× probe was prepared fresh the day of the assay: 1 μl of 5 mM probe to 39 μl PBS.

The following reagents were employed for the assays: BTK antibody: BD Biosciences 611117; 2nd Antibody: Goat anti-mouse-HRP, Santa Cruz, SC-2302; Streptavidin-HRP, Thermo, Cat#21130, 0.5 ml, 1-step Ultra TMB-ELISA Substrate, Thermo, cat#34028, 250 ml; Stop solution: 0.16 M H2SO4, Thermo, Cat# N600, 55 ml; Streptavidin coated plate: Thermo, cat#15500, 5 plate; DOHH2 control lysate; Wash buffer: 0.05% PBST; 1% BSA.

A standard curve was performed according to the following protocol:

1) Add 1 μl of 50× probe to 50 μl lysate (1 μg/μl), incubate at 37 C for 1 hr

2) Block Streptavidin coated plate with 1% BSA at RT for 1 hr, wash w/ PBST, 3×

3) Stop reaction by transfer samples onto ice for 10 min, then bring back to RT

4) Make series dilution of probe labeled DOHH2 lysate with 1% BSA in an order of 1 ug, 0.5 μg, 0.25 ug, 125 ng, 62.5 ng, 31.25 ng, 15.6 ng/μl to volume of 500

5) Add 100 μl probe treated lysate to each well in triplicate

6) Incubate at RT for 2 hrs

7) Wash with PBST, 7×

8) Add 2nd Ab-HRP, incubate at RT for 1 hr

9) Wash with 200 μl PBST, 5×

10) Add 100 μl TMB, incubate at RT for 15 min

11) Add 50 μl stop solution

12) Read at 450 nm

Protocol Format 1: (Streptavidin Coated Plate)

1) Dilute lysate to desired concentration with PBS to final volume of 500 μl

2) Add Biotinylated probe (final concentration 2.5 μM) to lysate, 37 C for 1 hr

3) Block streptavidin plate w/ 1% BSA, RT, 1 hr, wash w/ PBST 3×

4) Stop reaction by transfer samples onto ice for 10 min, then bring back to RT

5) Add 100 ul treated lysate (in triplicate) to streptavidin plate, RT, 2 hr w/ rocking

6) Wash with 200 μl PBST, 5×

7) Add 100 μl 2nd Ab-HRP, incubate at RT for 1 hr

8) Wash with 200 μl PBST, 3×

9) Add 100 μl TMB, incubate at RT for 10 min

10) Add 50 μl stop solution

11) Read at 450 nm

Protocol Format 2: (BTK antibody coated plate)

1) Coat 96 well-plate with 100 μl anti-BTK (1:1000 PBS), 4 C, o/n

2) Wash plate with PBST, 5×

3) Block plate for 1 hr at RT with rocking, wash w/ PBST 3×

4) Dilute lysate to desired concentration with PBS to final volume of 500

5) Add Biotinylated probe (final concentration 2.5 μM) to lysate, 37 C for 1 hr

6) Stop reaction by transfer samples onto ice for 10 min, then bring back to RT

7) Add 100 μl treated lysate (in triplicate) to streptavidin plate, RT, 2 hr w/ rocking

8) Wash with 200 μl PBST, 5×

9) Add streptavidin-HRP, incubate at RT for 1 hr with rocking

10) Wash with 200 μl PBST, 3×

11) Add 100 μl TMB, incubate at RT for 15 min

12) Add 50 μl stop solution

13) Read at 450 nm

Results:

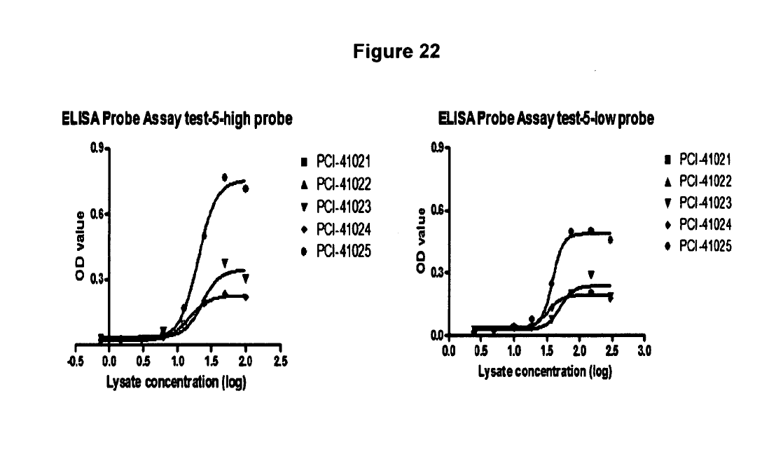

Exemplary results from the occupancy assays comparing the various probes are shown in FIG. 22 for high and low probe concentrations for the streptavidin assay. The data demonstrated that Compound I-5 exhibited the highest signal for binding to BTK out of the probes tested in the assay.

### Example 4

### Comparison of Kinase Inhibitor Activity of Compound I-5 and Parent Compound Ibrutinib

The ability of Compound I-5 to inhibit TEC family kinases and homologous tyrosine kinases was assayed in vitro using a standard kinase inhibition assay performed by Nanosyn. The following kinases were assayed according the table 4 below:

TABLE 4

| | | | | [Enzyme], | [ATP], | |
|---|---|---|---|---|---|---|
| Target | Vendor | Cat# | Lot | nM | μM | Inc time, hr |
| BLK | BPS | 40401 | 111102 | 0.4 | 20 | 1 |
| BMX | BPS | 40402 | 111031-5 | 1.82 | 75 | 1 |
| BTK | Millipore | 14552 | D7HN091U | 0.95 | 20 | 1 |
| ITK | Cama | 08-181 | 10CBS-1259E | 1 | 50 | 3 |
| JAK3 | INVITROGEN | PV3855 | 716673 | 2 | 2 | 3 |
| LCK | INVITROGEN | P3043 | 850070F | 0.4 | 50 | 1 |
| SRC | INVITROGEN | P3044 | 26726B | 3 | 25 | 1 |
| TEC | Millipore | 14-801 | D8PN044U | 4.6 | 12 | 1 |

Compounds were tested in singlicates using 12 pt (3×-dilutions) dose response format with top concentration of 10 mM or 1 mM.

Results are presented in Table 5. The data demonstrates that the Compound I-5 probe exhibited an IC50 profile for TEC family kinase inhibition similar to the parent compound ibrutinib.

TABLE 5

| | BLK | BMX | ITK | JAK3 | LCK | SRC | TEC | BTK |
|---|---|---|---|---|---|---|---|---|
| | IC50 | IC50 | IC50 | IC50 | IC50 | IC50 | IC50 | IC50 |
| Compound ID | (μM) | (μM) | (μM) | (μM) | (μM) | (μM) | (μM) | (μM) |
| Staurosporin | 0.00213 | 0.0128 | 0.00569 | 0.000825 | 0.00227 | 0.00662 | 0.0129 | 0.0189 |
| Ibrutinib | 0.000362 | 0.00109 | 0.0623 | 0.0849 | 0.00745 | 0.0242 | 0.000643 | 0.000248 |
| Compound I-5 | 0.00427 | 0.00557 | 0.198 | 4.87 | 0.00692 | 0.0363 | 0.00229 | 0.00102 |
| Compound I-5 | 0.00366 | 0.00442 | 0.187 | 5.34 | 0.00599 | 0.0338 | 0.00171 | 0.000755 |

### Example 5

### Development of Total BLK Protein Assays

The aim of this study was to demonstrate a method to screen antibodies against BLK to develop a sandwich immunoassay for quantifying total BLK in clinical samples on the MSD platform.

Materials used: MSD ELISA Conversion pack I (Catalog #K15A01-1) which contains: 96-well High Bind Plates, 96-well Standard Plates, SULFO-TAG™ NHS Ester, 150 nmoles, 4 Spin Columns, SULFO-TAG Labeled Streptavidin, 50 μg, SULFO-TAG Labeled Anti Mouse Antibody, 50 μg, SULFO-TAG Labeled Anti Rabbit Antibody, 50 μg, Blocker A Kit, 1 L, Blocker B, 2 g, Read Buffer T (4×), 200 mL.

The following BLK antibodies were tested:

TABLE 6

| | | | Species | Host | | | Application | |
|---|---|---|---|---|---|---|---|---|
| Cat. # | Vendor | Target | Reactivity | Species | Description | Formulation | tested | Conc. |
| AF2679 | R & D | BLK | H, M, Rt | Goat | BLK Affinity Purified | Lyoplilized | WB | 1 mg/ml |
| | | | | | goat | from | | |
| | | | | | Polyclonal Ab | PBS | | |
| | | | | | | solution | | |
| TA300020 | Origene | BLK | H, M | Rabbit | Purified BLK Rabbit | PBS with | WB | 0.25 mg/ml |
| | | | | | Polyclonal Antibody | Sodium | | |
| | | | | | | Azide | | |
| H00000640- | Novus | BLK | H, M, Rt | Mouse | IgG purified Mouse | PBS | WB, | |
| M02 | Biologicals | | | | | | ELISA | |
| ab37830 | Abcam | BLK | H, M | Rabbit | purified Rabbit | PBS with | WB, | 0.25 mg/ml |
| | | | | | Polyclonal | Sodium | ELISA, | |
| | | | | | | Azide | IHC | |

Solutions:

Blocking solution: 3% (w/v) MSD Blocker A in 1×PBS: 3 g Blocker A+100 mL PBS. Store at 4° C. for up to 14 days.

Wash Buffer: PBS-T (PBS+0.05% Tween-20)

Capture Antibody dilution buffer: PBS w/o Ca2+w/o Mg2+

Capture Antibody: Prepare 1 ml of 4 μg/ml solution of each antibody in Capture Antibody dilution buffer

Detection Antibody dilution buffer: 1% MSD Blocker A in 1×PBS: 10 mL Blocking solution+20 mL 1×PBS

Detection Antibody: Prepare 1-2 ml of 2 μg/ml solution of each antibody in 1% MSD Blocker A in 1×PBS

SULFO-TAG labeled Secondary Antibody: Prepare 3 ml each of 1 μg/ml solution of anti-mouse and anti-rabbit SULFO-TAG antibody in 1% MSD Blocker A in 1×PBS

Read Buffer: 1×MSD Read Buffer T: per plate 5 mL 4× Read Buffer T+15 mL H₂O Mix by inversion, do not vortex.

Standards: Prepare a 10 μg/ml stock solution of the recombinant BLK in 1% Blocker A. Then prepare the following dilutions for standard curve. Use a fresh tip for each dilution. Vortex well after each dilution.

Methods:

### Experiment 1

Aims: Screen Antibodies to identify a suitable antibody pair for further assay development

Protocol:

1. Solution coat one each of MSD high bind plate and Standard plate with 25 μl per well of the capture antibody solution (4 μg/ml) as per the plate layout (step 1). Add antibody solution to the corner of the wells and gently tap the plate to disperse the liquid evenly, seal the plate and incubate O/N at 4° C. without shaking.

2. Tap out the plate next day and add 150 μL per well blocking solution (3% [w/v] Blocker A). Seal and incubate with shaking 1 h or more at RT.

3. Wash plate 1× with ≧150 μL per well PBS-T. Tap dry.

4. Add 25 μL of recombinant protein diluted according to plate layout (step 2). Add solution to the bottom corner of the wells. Seal and shake plate at 300-500 rpm 1-2 h at RT.

5. Wash plate 3× with ≧150 μL per well PBS-T. Tap dry.

6. Add 25 μL of detection antibody solution according to the plate layout (step 3). Add Ab solution to the bottom corner of the wells. Seal and shake plate at 300-500 rpm 1 h at RT.

7. Wash plate 3× with ≧150 μL per well PBS-T. Tap dry.

8. Add 25 μL of SULFO-TAG anti-species detection Ab diluted to 1 μg/mL in detection antibody diluent according to the plate layout Step 3. Seal and shake plate at 300-500 rpm for 30 min at RT.

9. Wash plate 3× with ≧150 μL per well PBS-T. Tap dry

10. Add 150 μL per well 1× Read Buffer T (diluted in H20). Avoid bubbles—use reverse pipetting when adding Read Buffer. Read plate immediately in SI2400.

Results:

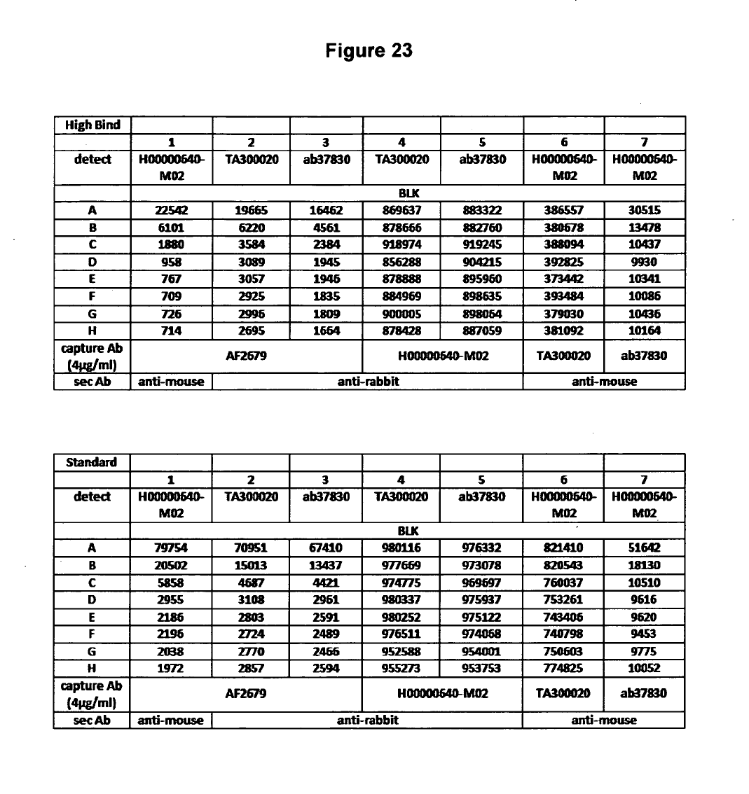

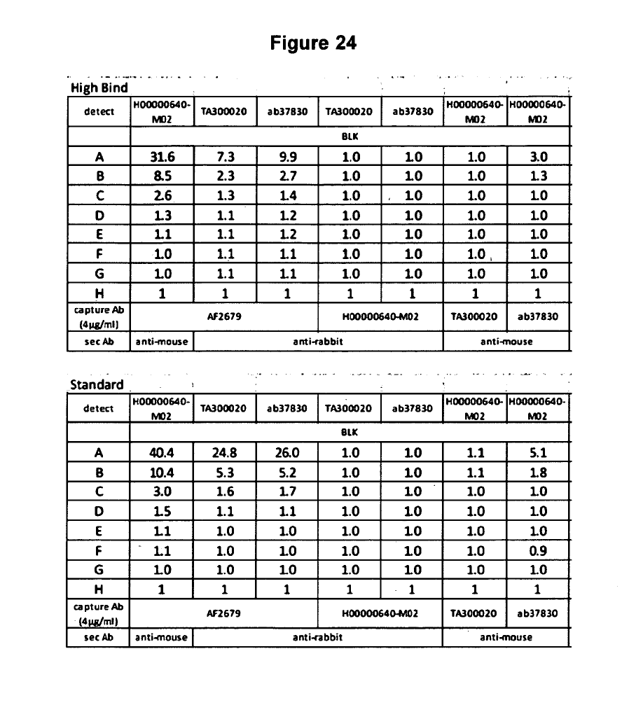

Raw signals for different antibody pairs are shown in FIG. 23. Signal to background (S/B) for different antibody pairs are shown in FIG. 24, the highest S/B ratio was observed for H00000640-MO2. A nice titration of signal and S/B with protein concentration was observed for four antibody pairs for BLK. The antibody pair (AF2679 as capture and H00000640-MO2 as detect) and orientation with the highest S/B ratio was selected for further optimization of total BLK assay. For the same antibody pairs, higher S/B ratio was observed on standard plates.

### Experiment 2

Aims:

1) Develop assay conditions for BLK assay using AF2679 as capture and H00000640-MO2 as detection antibody

2) Test positive (DOHH2) and negative (Jurkat) cell lysates for BLK expression

Antibodies were conjugated to SULFO-TAG™ NHS Ester according to manufacturers instructions (Meso Scale Discovery).

Protocol:

1. Solution coat MSD Standard plate with 25 μl per well of the capture antibody solution (4 μg/ml) as per the plate layout. Add antibody solution to the corner of the wells and gently tap the plate to disperse the liquid evenly seal the plate and incubate O/N at 4° C. without shaking. 2. Tap out the plate next day and add 1504 per well blocking solution (5% [w/v] Blocker A). Seal and incubate with shaking 1 h or more at RT.

3. Wash plate 1× with ≧150 μL per well PBS-T. Tap dry.

4. Add 25 μL of recombinant protein diluted according to plate layout. Add solution to the bottom corner of the wells. Seal and shake plate at 300-500 rpm 1-2 h at RT.

5. Wash plate 3× with ≧150 μL per well PBS-T. Tap dry.

6. Add 25 μL of detection antibody solution according to the plate layout. Add Ab solution to the bottom corner of the wells. Seal and shake plate at 300-500 rpm 1 h at RT.

7. Wash plate 3× with ≧150 μL per well PBS-T. Tap dry.

8. Add 150 μL per well 1× Read Buffer T (diluted in H20) to plate 2. Avoid bubbles—use reverse pipetting when adding Read Buffer. Read plate immediately in S12400.

9. Add 25 μL of SULFO-TAG anti-species detection Ab diluted to 1 μg/mL in detection antibody diluent according to the plate layout to plate 1. Seal and shake plate at 300-500 rpm for 30 min at RT.

10. Wash plate 1 3× with ≧150 μL per well PBS-T. Tap dry.

11. Add 150 μL per well 1× Read Buffer T (diluted in H20). Avoid bubbles—use reverse pipetting when adding Read Buffer. Read plate immediately in S12400.

Results:

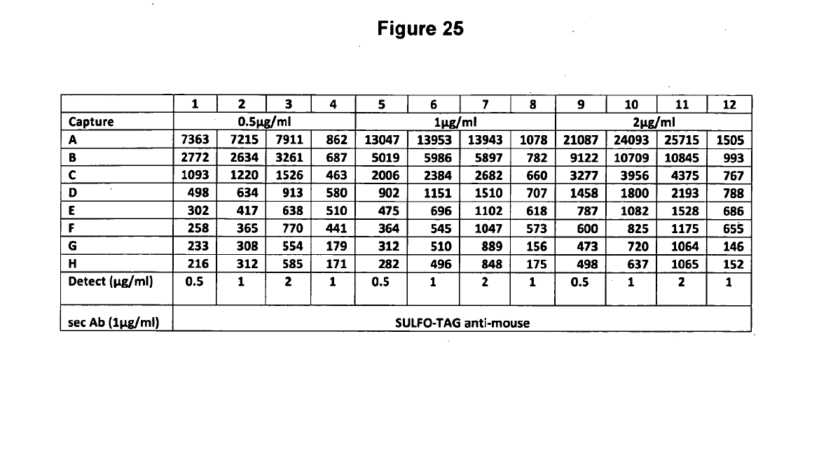

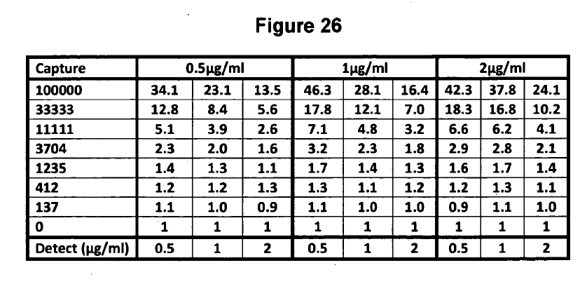

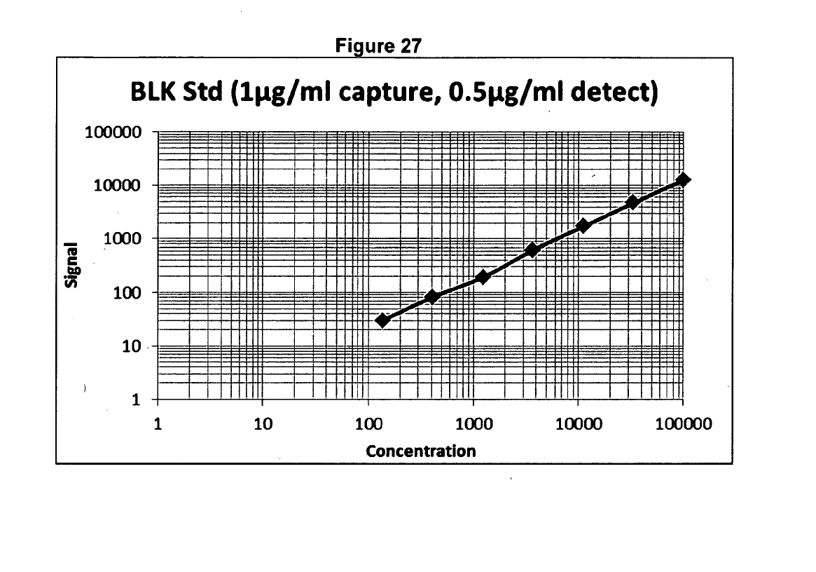

Raw signals for the assay are shown in FIG. 25. Signal to background (S/B) for the assay is shown in FIG. 26. The BLK assay demonstrated signal titration with protein concentration. The signal values for recombinant BLK protein using 1 μg/ml capture antibody and 0.5 μg/ml detection antibody are plotted in FIG. 27. Positive to negative ratio for BLK in cell lysates is shown in table below:

TABLE 7

| Capture (ug/ml) | 0.5 | 1 | 2 |
|---|---|---|---|
| 10 μg | 1.49 | 1.52 | 1.91 |
| 5 μg | 1.35 | 1.27 | 1.45 |
| 2.5 μg | 1.05 | 1.15 | 1.17 |

| | |
|---|---|
| Detect (ug/ml) | 1 |

A signal window of up to 46 fold was observed for BLK assay with 1 μg/ml capture antibody (AF2679, R&D) and 0.5 μg/ml (H00000640-MO2, Novus Biologicals) detection antibody. The dynamic range of the assay appears to be ˜3.5 logs. P/N ratio of ˜2 fold for BLK was observed in cell lysates using partially optimized conditions.

### Example 6

### ITK and BLK Protein Occupancy Assays

The aim of the study was to optimize the probe required for ITK and BLK occupancy assay on MSD Streptavidin plates and to demonstrate the assay performance using drug treatment of lysates. The purpose of the assay is to determine the relative amount of ITK and BLK that has not been bound by the covalent inhibitor hereafter referred to as the “drug”. The “probe” consists of the drug linked to biotin via a long chain linker. Labeling of samples with the probe allows for the detection of ITK and BLK not occupied by drug. The assay format that has previously been successfully used with BTK is capturing the probe labeled protein on Streptavidin plates and detecting using anti-protein antibodies. A similar format was tested for ITK and BLK occupancy assays,

Materials:

Standard Streptavidin plate (5-pack) Catalog #L15SA-2, Read Buffer T (50 mL) Catalog # R92TC-3, SULFO-TAG Goat Anti-mouse (50 μg) Catalog# R32AC-5, SULFO-TAG Goat Anti-rabbit (50 μg) Catalog# R32AB-5, SULFO-TAG streptavidin (50 ug) Catalog# R32AD-5, MSD Blocker A Catalog# R93AA-2, Protease inhibitor cocktail (e.g., Thermo/Pierce Halt™ Protease Inhibitor Cocktail EDTA free Cat #87785 or Roche Complete Mini Protease inhibitor tablet, Cat #1836170), Positive Control lysates from ITK and BLK expressing cell line (DOHH2 and JURKAT), Negative Control lysates (THP-1 cells), BLK, ITK inhibitor drug ibrutinib “PCI”; Biotinylated probe Compound I-5 “probe”.

Solutions:

Blocking solution: 3% (w/v) MSD Blocker A in 1× Tris Wash buffer: 3 g Blocker A+100 mL 1× Tris wash Buffer. Store at 4° C. for up to 14 days. Blocking solution may also be prepared in PBS-T

Wash Buffer: 1×MSD Tris wash buffer: 50 mL 10× Tris Wash buffer+450 mL H₂O (150 mM NaCl 50 mM Tris-HCl pH 7.5 0.02% Tween-20). PBS-T can also be used as a wash buffer

Detection Antibody dilution buffer: 1% MSD Blocker A in 1× Tris Wash Buffer: 10 mL Blocking solution+20 mL 1× Tris Wash Buffer or 10 mL Blocking solution+20 mL PBS-T

Read Buffer: 1×MSD Read Buffer T: per plate 5 mL 4× Read Buffer T+15 mL H2O

Cell lysates: Lysates prepared by repeated freeze-thaw of cell pellets resuspended in PBS+ protease inhibitors.

Assay Buffer: 1% blocker in Tris wash buffer+ protease inhibitors

### Experiment 1

### Optimizing Probe Concentration

Aims:

Determine optimal probe concentration required and compare different probe: lysate ratios

Samples:

Positive control: DOHH2 and Jurkat cell lysates

Negative control: DOHH2 and Jurkat cells treated with 1 uM PCI

Dilute DOHH2 only and DOHH2+PCI lysates to 600; 300; 150; 75; 37.5; 18.75 ug/mL in assay buffer

Dilute probe to 6000; 1500; 375; 93.8; 23.4; 5.9 and 1.5 nM (50× concentration)

Add 1 μL 100× probe to 50 μL diluted lysates in a 96 well polypropylene plate, incubate

Protocol:

1) Block MSD plates with 150 μl 3% Blocker A for I-3 h at RT with shaking at 900 rpm.

2) Incubate titration of protein lysates with titration of probe in 50 ul volume in a separate 96 well polypropylene plate for 1 h with shaking

3) Wash MSD plate 2× with wash buffer.

4) Transfer 30 ul of probe lysate mix to MSD plates as per the plate layout and incubate for 1 h.

5) Wash plate 3× with 150 ul wash buffer.

6) Add 25 ul of anti-BLK or anti-ITK antibody diluted to 2 ug/ml in 1% Blocker A. Incubate for 2 h at RT with shaking at 900 rpm.

7) Wash plates 3× with 150 μl wash buffer.

8) Add 25 ul of SULFO-TAG labeled anti-rabbit antibody diluted to 1 ug/ml in 1% Blocker A. Incubate for 1 h at RT with shaking at 900 rpm.

9) Wash plates 3× with MSD wash buffer, add 150 μL 1× Read Buffer and read immediately in sector Imager.

Results:

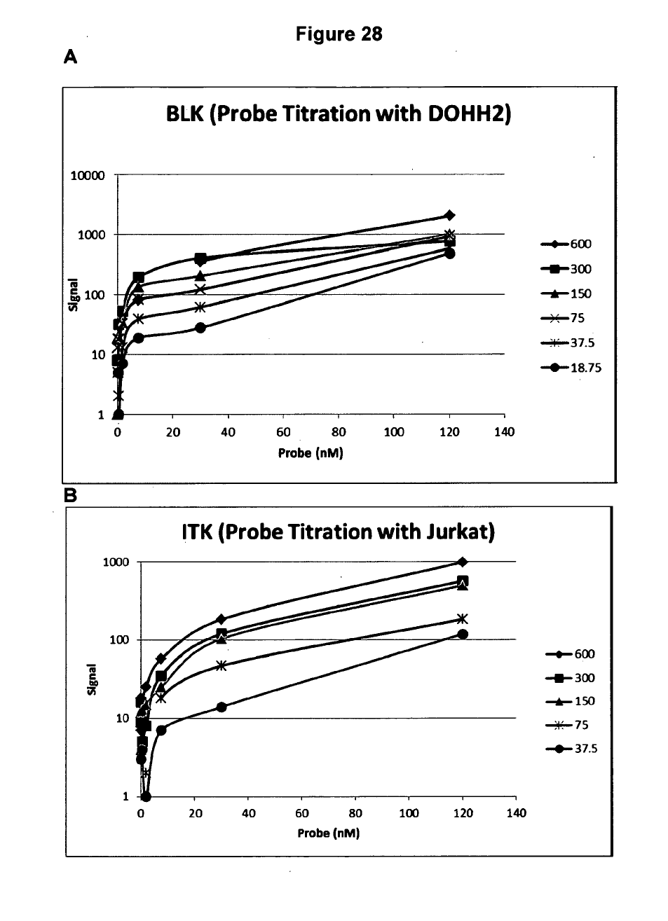

Results are presented in FIG. 28 for the BLK and ITK probe assays. It was observed that the signal titrates with the protein concentration in lysate used for both BTK and ITK assays. The signal increases with increasing probe concentration for positive control lysates and plateau beyond about 30 nM. The background signal is low for the negative control (Cell lysate+drug) samples, with very little increase at 120 nM probe. A probe concentration of 50 nM was recommended for further experiments.

### Experiment 2

### Inhibition with Drug Titration Series

Aim:

Carry out inhibition experiment with titration series of drug then label lysates with 50 nM probe.

Samples:

Positive control: DOHH2 and Jurkat lysate at 1 mg/mL in assay buffer.

Negative control: THP-1 lysate at 1 mg/mL in assay buffer.

Dilute lysates to 500; 250 and 125 μg/mL in assay buffer

Prepare a dilution series of PCI. The 100×PCI concentrations are: 100; 25; 6.25; 1.56; 0.39; 0.09 μM; treat lysates with PCI in a polypropylene plate for 1 h e.g., 100 μL lysate+1 μL 100×PCI solution; after the PCI inhibition add probe to all the samples to a final concentration of 50 nM, incubate for 1 h at RT with shaking

Protocol:

1) Block MSD standard streptavidin plate with 150 μL per well 3% Blocker A 1 h at RT or overnight at 4° C.

2) Wash plate 1× with ≧150 μL per well wash buffer. Tap dry.

3) Add 30 μL of probe labeled lysate according to the plate layout. Add lysate solution to the bottom corner of the wells. Seal and shake plate 1 h at RT.

4) Wash plate 3× with ≧150 μL per well wash buffer. Tap dry.

5) Add 25 μL per well of anti-ITK antibody diluted to 2 μg/mL in 1% (w/v) Blocker A. Seal and incubate for 2 h at RT with shaking at 900 rpm.

6) Add 25 μL per well of SULFO-TAG conjugated anti-species antibody diluted to 2 μg/mL in 1% (w/v) Blocker A. Seal and shake plate 1 h at RT.

7) Wash plate 3× with ≧150 μL per well 1× Tris wash buffer. Tap dry

8) Add 150 μL per well 1× Read Buffer T (diluted in H20). Avoid bubbles—use reverse pipetting when adding Read Buffer. Read plate in SI2400.

Results:

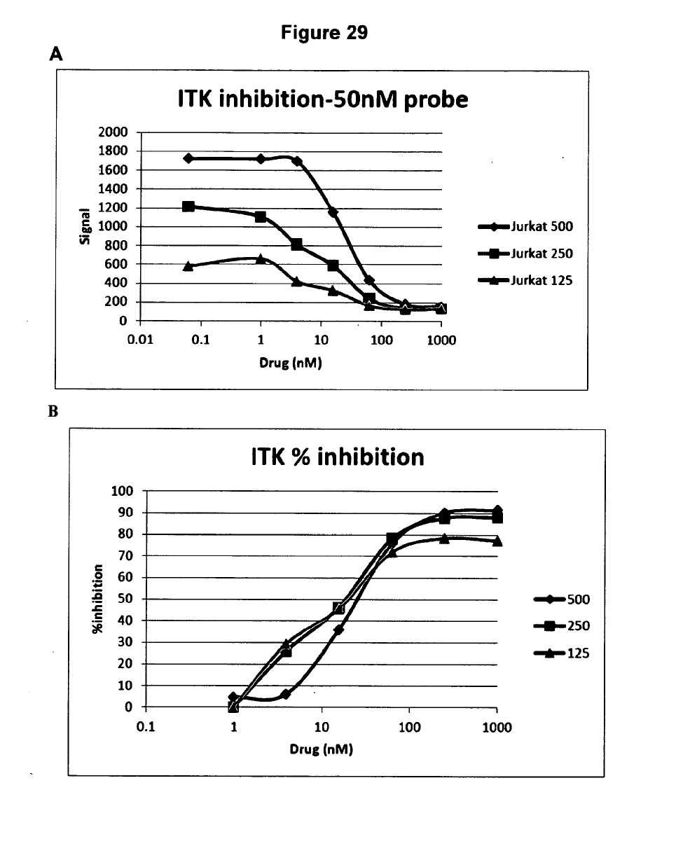

Results for the ITK probe assay are presented in FIG. 29 and in Table 8 below. The signal of negative control cell line, DOHH2, was observed at background levels at all concentrations of drug. The drug treated positive control, Jurkat cell lysates, demonstrated decrease in signal with increasing drug concentration. The % inhibition was irrespective of the lysate concentration used. The assay reproducibility was very good with average % CV<5%.

TABLE 8

| | ITK assay (Jurkat lysate) | | | | | |
|---|---|---|---|---|---|---|
| Drug | 500 μg/ml | | 250 μg/ml | | 125 μg/ml | |
| (nM) | Av signal | % CV | Av signal | % CV | Av signal | % CV |
| un- | 1805 | 0.63 | 1098 | 3.5 | 596 | 2.1 |
| treated | | | | | | |
| DMSO | 1721.5 | 4.15 | 1217 | 0.2 | 581.5 | 1.1 |
| 1.0 | 1720.5 | 4.81 | 1107 | 1.1 | 662 | 1.3 |
| 3.9 | 1696 | 0.67 | 814.5 | 2.0 | 422 | 2.3 |
| 15.6 | 1158 | 1.34 | 589 | 1.2 | 324 | 1.7 |
| 62.5 | 433.5 | 2.12 | 240 | 2.4 | 167.5 | 0.4 |
| 250 | 180.5 | 1.96 | 135.5 | 2.6 | 129 | 1.1 |
| 1000 | 155 | 0.00 | 134 | 2.1 | 135.5 | 13.0 |

### Experiment 3

### Repeat of Inhibition of ITK in PBMC Lysates

Aim:

Carry out inhibition experiment with titration series of drug then label lysates with 50 nM probe.

Samples:

Positive control: DOHH2, Jurkat and PBMC lysate at 1 mg/mL in assay buffer.

Negative control: THP-1 lysate at 1 mg/mL in assay buffer.

Dilute lysates to 500; 250 and 125 μg/mL in assay buffer

Prepare a dilution series of PCI. The 100×PCI concentrations are: 100; 25; 6.25; 1.56; 0.39; 0.09 μM; treat lysates with PCI in a polypropylene plate for 1 h e.g., 100 μL lysate+1 μL 100×PCI solution; after the PCI inhibition add probe to all the samples to a final concentration of 50 nM, incubate for 1 h at RT with shaking

Protocol:

1) Block MSD standard streptavidin plate with 150 μL per well 3% Blocker A 1 h at RT or overnight at 4° C.

2) Wash plate 1× with ≧150 μL per well wash buffer. Tap dry.

3) Add 30 μL of probe labeled lysate according to the plate layout. Add lysate solution to the bottom corner of the wells. Seal and shake plate 1 h at RT.

4) Wash plate 3× with ≧150 μL per well wash buffer. Tap dry.

5) Add 25 μL per well of anti-ITK antibody diluted to 2 μg/mL in 1% (w/v) Blocker A. Seal and incubate for 2 h at RT with shaking at 900 rpm.

6) Wash plates 3× with 150 μl wash buffer.

7) Add 25 μL per well of SULFO-TAG conjugated anti-species antibody diluted to 2 μg/mL in 1% (w/v) Blocker A. Seal and shake plate 1 h at RT.

8) Wash plate 3× with ≧150 μL per well 1× Tris wash buffer. Tap dry

9) Add 150 μL per well 1× Read Buffer T (diluted in H20). Avoid bubbles—use reverse pipetting when adding Read Buffer. Read plate in SI2400.

Results:

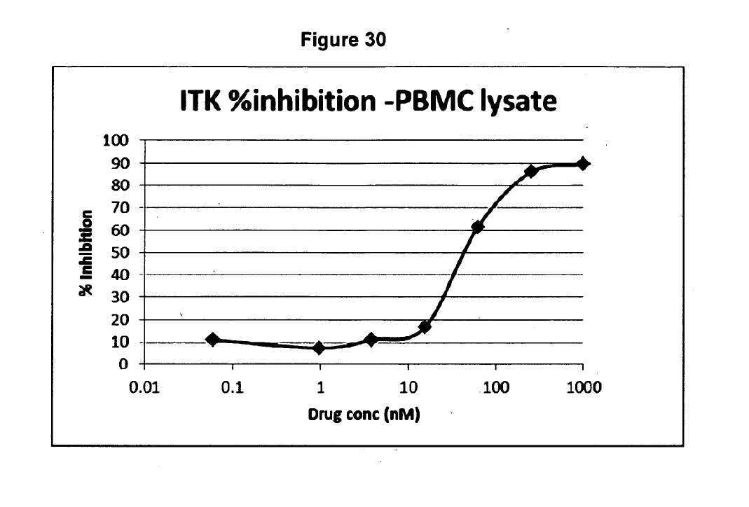

Results for the ITK probe assay are presented in FIG. 30 and in Table 9 below. A dose dependent decrease in signal of ITK was observed with PBMC lysates indicating inhibition of ITK by the drug in PBMC lysates. The reproducibility of ITK assay was very good again with % CV<5%.

TABLE 9

| | ITK inhibition (PBMC lysates) | | |
|---|---|---|---|
| Drug (nM) | Av signal | % CV | % inhibition |
| Untreated | 998 | 5 | |
| DMSO | 889 | 3 | 11 |
| 0.98 | 925 | 3 | 7 |
| 3.91 | 889 | 1 | 11 |
| 15.63 | 832 | 1 | 17 |
| 62.50 | 384 | 1 | 62 |
| 250 | 139 | 3 | 86 |
| 1000 | 106 | 5 | 89 |

### Example 7

### ITK Protein Occupancy Assay

The ELISA SULFO-TAG ITK probe assay, based upon an electrochemical stimulation, will be used to determine the relative amount of ITK that has not been bound by ibrutinib. Ibrutinib binds to the active site of ITK and forms a disulfide bond with a cysteine residue (ITK-Cys442). Compound I-5 is a probe that consists of ibrutinib linked to biotin via a long chain linker. The collected protein lysates are labeled with Compound I-5. Labeling of samples with the probe allows for the detection of ITK not occupied by drug. The probe conjugated with ITK (and un-conjugated probe) is captured by the Streptavidin (SA) plate that is subsequently incubated with mouse anti-ITK (BD#550503) and SULFO-TAG conjugated anti-mouse antibody (MSD, cat#R32AC-5). The SULFO-TAG labels emit light upon electrochemical stimulation initiated at the electrodes in each well and signal is measured. A larger the signal correlates to more unoccupied ITK sites of a sample while a lower signal correlates to more ibrutinib occupied ITK sites.

The baseline for ITK occupancy was set at the pre-dose Cycle1 Day 1 sample and the percent of ITK occupancy at the prescribed time points was calculated by this baseline value. This percentage was used as the pharmacodynamic output and compared between different dose cohorts of patients. Thus the relationship between the ibrutinib dose cohort and ITK occupancy was defined.

ITK occupancy in CLL patients on a phase II clinical trial of ibrutinib was determined. Samples were tested immediately prior to receiving ibrutinib and after eight days of daily oral administration (420 mg/day). PBMC were collected, and lysed by freeze-thawing 4 times. After the final thaw, cells were centrifuged at 16,000 g for 10 min at 4 C to pellet insoluble material. Protease inhibitors were added to the protein lysates, and the protein lysates are labeled with a biotinylated derivative of drug, Compound I-5, for 1 hr at RT and are added to a Streptavidin coated plate (MSD, cat #L15SA-2) that is blocked for 1 hr with blocking solution. After 1 hr of incubation, the plates were washed 3× and followed by an incubation of mouse anti-ITK (BD#550503) for another hr. The plate was washed 3× and incubated for 1 hr with SULFO-TAG conjugated anti-mouse antibody (MSD, cat#R32AC-5), washed, and read on a S12400 for 3 min according to manufacturer's instructions. The data revealed between 40 and 80% of ITK is covalently bound by ibrutinib similar that achieved in vitro.

What is claimed is:

1. A kit for determining drug target occupancy in a patient receiving a TEC family kinase inhibitor therapy, comprising a probe having the structure of Formula (II) comprising: 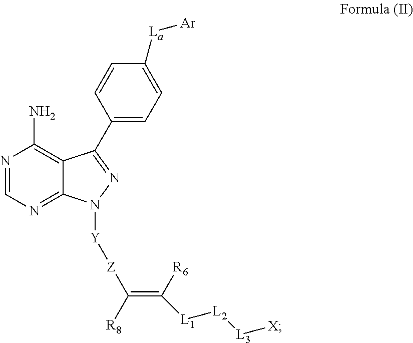
wherein:
Lₐ is CH₂, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), S(O)ₙ, OS(O)ₙ, NHS(O)ₙ, where n is 1 or 2;
R₆ and R₈ are independently selected from H, optionally substituted alkyl, or optionally substituted heteroalkyl;
L₁ is optionally substituted alkyl or optionally substituted heteroalkyl;
L₂ is a bond, optionally substituted heterocycloalkyl, or —N(H)C(O)(CH₂)ₘC(O)N(H), where m is 2-6;
L₃ is optionally substituted alkyl or optionally substituted heteroalkyl; and X is a detectable label, wherein the probe binds to a TEC family kinase.

2. The kit of claim 1, wherein X is:

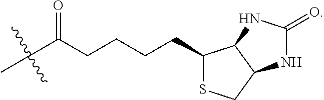 [SMILES: *C(=O)CCCC[C@@H]1SC[C@@H]2NC(=O)N[C@@H]21]

3. The kit of claim 1, wherein the probe has the structure of:

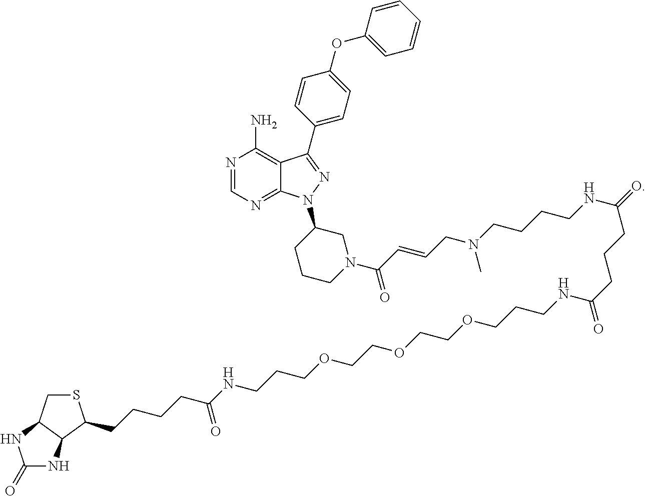 [SMILES: CN(C/C=C/C(=O)N1CCC[C@@H](n2nc(-c3ccc(Oc4ccccc4)cc3)c3c(N)ncnc32)C1)CCCCNC(=O)CCCC(=O)NCCCOCCOCCOCCCNC(=O)CCCC[C@@H]1SC[C@@H]2NC(=O)N[C@@H]21]

4. The kit of any of claims 1-3, wherein the probe binds to Bruton's tyrosine kinase (BTK), ITK, TEC, BMX, TXK or BLK.

5. The kit of any of claims 1-4, further comprising one or more solid supports selected from among a plate, a microplate, a bead or a plurality of beads.

6. The kit of claim 5, wherein the solid support is coated with a capture agent to form a coated solid support, wherein the capture agent binds to the probe.

7. The kit of claim 6, wherein the capture agent is streptavidin or an antibody.

8. The kit of any of claims 1-7, further comprising a primary detection agent, and optionally, a secondary detection agent that binds to the primary detection agent.

9. The kit of claim 8, wherein the primary detection agent or secondary detection agent comprises an antibody, a bead, a dye, a label, a tag, a fluorophore, or any combination thereof.

10. The kit of claim 9, wherein the primary detection agent is an anti-BTK antibody, an anti-ITK antibody, an anti-TEC antibody, an anti-TXK antibody, an anti-BMX antibody, or an anti-BLK antibody.

11. The kit of any of claims 8-10, wherein the primary or secondary detection agent is conjugated to an electrochemiluminescent tag.

12. A method for determining drug target occupancy in a patient receiving a TEC family kinase inhibitor therapy, comprising:
(a) contacting a sample comprising a TEC family kinase with a probe to form a probe-bound kinase, wherein the sample is obtained from the patient following administration of at least one dose of an irreversible TEC family kinase inhibitor;
(b) detecting the amount of probe-bound kinase in the sample; and
(c) determining target occupancy of the TEC family kinase based on the amount of probe-bound kinase detected in the sample,
wherein the probe has the structure of Formula (II) comprising: 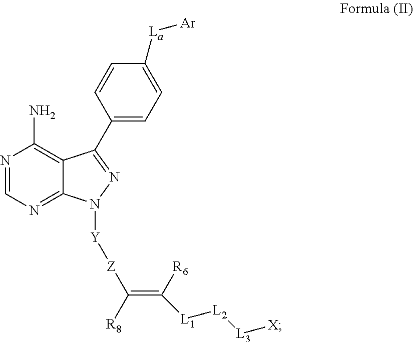
wherein:
Lₐ is CH₂, O, NH or S;
Ar is optionally substituted aryl or optionally substituted heteroaryl;
Y is optionally substituted alkyl, optionally substituted heteroalkyl, optionally substituted cycloalkyl, optionally substituted heterocycloalkyl, optionally substituted aryl, or optionally substituted heteroaryl;
Z is C(O), OC(O), NHC(O), C(S), S(O)ₙ, OS(O)ₙ, NHS(O)ₙ, where n is 1 or 2;
R₆ and R₈ are independently selected from H, optionally substituted alkyl, or optionally substituted heteroalkyl;
L₁ is optionally substituted alkyl or optionally substituted heteroalkyl;
L₂ is a bond, optionally substituted heterocycloalkyl, or —N(H)C(O)(CH₂)ₘC(O)N(H), where m is 2-6;
L₃ is optionally substituted alkyl or optionally substituted heteroalkyl; and
X is a detectable label.

13. The method of claim 12, wherein X is:

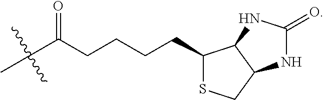 [SMILES: *C(=O)CCCC[C@@H]1SC[C@@H]2NC(=O)N[C@@H]21]

14. The method the method of claim 13, wherein the probe has the structure of:

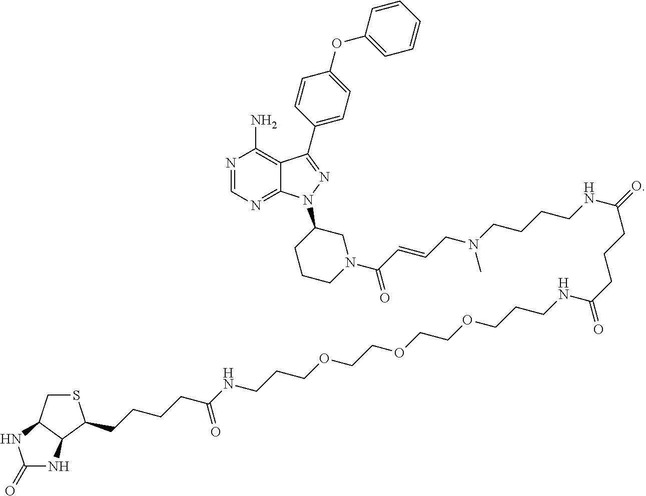 [SMILES: CN(C/C=C/C(=O)N1CCC[C@@H](n2nc(-c3ccc(Oc4ccccc4)cc3)c3c(N)ncnc32)C1)CCCCNC(=O)CCCC(=O)NCCCOCCOCCOCCCNC(=O)CCCC[C@@H]1SC[C@@H]2NC(=O)N[C@@H]21]

15. The method of any of claims 12-14, wherein determining target occupancy comprises i) determining the number of binding sites not bound to the TEC family kinase inhibitor based on the amount of probe-bound kinase detected in the sample and ii) comparing said number to the total amount of active TEC family kinases in the sample.

16. The method the method of any of claims 12-15, further comprising determining or modifying a therapeutic regimen based on the target occupancy of the TEC family kinase.

17. A method for monitoring drug target occupancy in a patient receiving a TEC family kinase inhibitor therapy, comprising performing the method of any of claims 12-16 at two or more time points over the course of the therapy.

18. The method the method of any of claims 16-17, further comprising: i) increasing the dosage of the TEC family kinase inhibitor if the target occupancy is less than about 50%, ii) decreasing the dosage of the TEC family kinase inhibitor if the target occupancy is above at least about 70%, iii) maintaining the same therapeutic regimen of the TEC family kinase inhibitor or iv) discontinuing the therapeutic regimen.

19. The method of any of claims 12-18, further comprising determining the efficacy of the TEC family kinase inhibitor therapy based on the target occupancy, wherein the TEC family kinase inhibitor is i) effective when the occupancy of the TEC family kinase is at least about 70% or ii) ineffective when the occupancy of the TEC family kinase is less than about 50%.

20. The method of any of claims 12-19 above, wherein the TEC family kinase inhibitor an inhibitor of a Bruton's tyrosine kinase (BTK).

21. The method of any of claims 12-20, wherein the TEC family kinase inhibitor is ibrutinib.

22. The method of any of claims 12-21, further comprising capturing probe-bound kinase with a capture agent.

23. The method of claim 22, wherein the capture target is streptavidin or an antibody.

24. The method of claim 22 or 23, wherein the capture target is attached to a solid support.

25. The method claim 24, wherein the solid support is a plate, a microplate, a bead or a plurality of beads.

26. The method of any of claims 12-25, further comprising contacting the probe-bound kinase with a primary detection agent, and optionally, a secondary detection agent that binds to the primary detection agent.

27. The method of claim 26, wherein the primary detection agent or secondary detection agent comprises an antibody, a bead, a dye, a label, a tag, a fluorophore, or any combination thereof.

28. The method of any of claims 26-27 above, wherein the primary detection agent is an anti-BTK antibody, an anti-ITK antibody, an anti-TXK antibody, anti-TEC antibody, anti-BMX antibody, or anti-BLK antibody.

29. The method of any of claims 26-28, wherein the primary or secondary detection agent is conjugated to a chemiluminescent tag.

30. The method of any of claims 12-29, wherein patient is suffering from a cancer.

31. The method of claim 30, wherein cancer is Hodgkin's lymphoma or a non-Hodgkin's lymphoma (NHL), chronic lymphocytic leukemia (CLL), small lymphocytic lymphoma (SLL), mantle cell leukemia (MCL), follicular lymphoma (FL), diffuse large B-cell lymphoma (DLBCL), Waldenström macroglobulinemia or multiple myeloma (MM).

32. The method of any of claims 12-31, wherein the sample is a blood sample, a lymph sample or tumor biopsy sample.
